(19) United States
(12) Patent Application Publication
(10) Pub. No.: US 2026/0124300 A1
(43) Pub. Date: May 7, 2026

(54) COMBINATION TREATMENT OF AN ANTI-CD20/ANTI-CD3 BISPECIFIC ANTIBODY AND CHEMOTHERAPY

(71) Applicants: Hoffmann-La Roche Inc., Little Falls, NJ (US); Genentech, Inc., South San Francisco, CA (US)

(72) Inventors: Mark Francis DIXON, Welwyn Garden City (GB); Maria FILIPPOU-FRYE, Brisbane, CA (US); Ritika Hari JAGTIANI, Milpitas, CA (US); Linda Maria LUNDBERG, Binningen (CH); Stephen James SIMKO, III, San Francisco, CA (US)

(21) Appl. No.: 19/372,448

(22) Filed: Oct. 29, 2025

Related U.S. Application Data
(60) Provisional application No. 63/715,214, filed on Nov. 1, 2024.
(60) Provisional application No. 63/727,563, filed on Dec. 3, 2024.

(51) Int. Cl. A61K 39/395; A61K 31/138; A61K 31/167; A61K 31/185; A61K 31/282; A61K 31/573; A61K 31/675; A61K 31/7048; A61K 39/00; A61P 35/00

(52) U.S. Cl. CPC A61K 39/39558; A61K 31/138; A61K 31/167; A61K 31/185; A61K 31/282; A61K 31/573; A61K 31/675; A61K 31/7048; A61P 35/00; A61K 2039/505; A61K 2039/545

(57) ABSTRACT

The present invention relates to methods of treating B-cell lymphomas, e.g., primary refractory or relapsed diffuse large B-cell lymphoma (DLBCL), by administering an anti-CD20/anti-CD3 bispecific antibody (e.g., glofitamab) and in combination with one or more anti-CD20 antibodies (e.g., obinutuzumab and/or rituximab) and one or more chemotherapeutic agents selected from ifosfamide, carboplatin, and/or etoposide.

### SEQUENCE LISTING

The instant application contains a Sequence Listing which has been submitted electronically in XML format and is hereby incorporated by reference in its entirety. Said XML copy, created on Oct. 1, 2025, is named 51177-061003_Sequence_Listing_10_125.xml and is 52,557 bytes in size.

### FIELD OF THE INVENTION

The present invention relates to methods of treating B-cell lymphomas, e.g., primary refractory or relapsed diffuse large B-cell lymphoma (DLBCL), by administering an anti-CD20/anti-CD3 bispecific antibody (e.g., glofitamab) in combination with one or more anti-CD20 antibodies (e.g., obinutuzumab and/or rituximab) and chemotherapeutic agents (e.g., ifosfamide, carboplatin, and etoposide).

### BACKGROUND OF THE INVENTION

Non-Hodgkin lymphoma (NHL) is the most common hematologic malignancy in the world (Bray et al. 2018). The most common subtype of NHL of B-cell origin (Sun et al. Am. J. Clin. Pathol. 138:429-434, 2012; Al-Hamadani et al. Am. J. Hematol. 24:4785-4797, 2015), DLBCL is an aggressive NHL with a median survival of <1 year in untreated patients (Rovira et al. Ann. Hematol. 94:803-812, 2015). Despite its aggressive disease course, approximately 50%-70% of patients may be cured with the current standard-of-care treatment that consists of rituximab, a monoclonal antibody targeting CD20, in combination with cyclophosphamide, doxorubicin, vincristine, and prednisone (R-CHOP) chemotherapy (Flowers et al. C A Cancer J. Clin. 60:393-408 2010; Tilly et al. Ann. Oncol. 26(Suppl 5):v116-125 2015; NCCN Clinical Practice Guidelines in Oncology, 2020).

Nevertheless, R-CHOP is found to be inadequate in 30%-50% of patients because of either primary refractoriness, defined as failure to achieve a complete response (CR) after first-line therapy with rituximab plus an anthracycline (Vardhana et al. Br. J. Haematol. 176:591-599, 2017), or relapse after achieving a CR. For patients who are not cured by first-line therapy and who are medically able to tolerate intensive therapy, high-dose salvage chemoimmunotherapy followed by ASCT offers a second chance for long-term remission. Approximately half of patients with relapsed DLBCL are refractory to salvage chemoimmunotherapy (Gisselbrecht et al. J. Clin. Oncol. 28:4184-4190, 2010) and are thus unable to proceed to ASCT. In addition, for a subgroup of patients with DLBCL, CAR-T therapy is an available treatment option, particularly for patients that have primary refractory disease, or have relapsed within 12 months of initial chemoimmunotherapy (Kamdar et al. 2022, Lancet 399, 2294-2308; Locke et al. 2022, N Engl J Med 386, 640-54). Given that the median EFS observed in both studies was less than 1 year, continued opportunity for optimization of therapeutic regimens involving CAR-T therapy remains. Therefore, a significant clinical need exists for improved salvage immunochemotherapy regimens for patients with relapsed or refractory (R/R) DLBCL.

### SUMMARY OF THE INVENTION

The invention features methods for treating an individual having a diffuse-large B-cell lymphoma (DLBCL) (e.g., a relapsed and/or refractory DLBCL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS)) by administering to the individual a treatment comprising glofitamab in combination with rituximab, ifosfamide, carboplatin and etoposide (i.e., glofit-R-ICE).

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in complete response (CR) rate of a plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.

In some embodiments, the improvement in CR rate is between 8% and 44% (e.g., between 8% and 40%, between 8% and 30%, between 8% and 20%, between 10% and 44%, between 20% and 44%, between 30% and 44%, between 20% and 30%, between 10% and 40%, between 10% and 15%, between 15% and 20%, between 20% and 25%, between 25% and 30%, between 30% and 35%, or between 35% and 40%; about 8%, 10%, 15%, 20%, 25%, 30%, 35%, 40%, or 44%). In some embodiments, the improvement in CR rate is an increase of about 28%.

In some embodiments, the improvement in CR rate is statistically significant.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in a CR rate of between 45% and 83% (e.g., between 45% and 80%, between 45% and 70%, between 45% and 60%, between 45% and 50%, between 55% and 83%, between 60% and 83%, between 70% and 83%, between 50% and 55%, between 55% and 60%, between 60% and 65%, between 65% and 70%, between 75% and 80%, between 50% and 70%, between 60% and 80%, or between 60% and 70%; about 45%, 50%, 55%, 60%, 65%, 70%, 75%, 80%, or 83%). In some embodiments, the CR rate is about 66%. In some embodiments, the CR rate is about 67%. In some embodiments, the CR rate is about 69%.

In some embodiments, the CR rate is the proportion of individuals whose best overall response is a CR as determined by positron emission tomography-computed tomography (PET-CT) or positron emission tomography-magnetic resonance imaging (PET-MRI).

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in overall response (OR) rate of a plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.

In some embodiments, the improvement in OR rate is between 1% and 29% (e.g., between 1% and 20%, between 1% and 10%, between 10% and 29%, between 20% and 29%, between 1% and 5%, between 5% and 10%, between 10% and 15%, between 15% and 20%, between 20% and 25%, between 25% and 29%, between 5% and 15%, between 15% and 29%, or between 10% and 20%; about 1%, 5%, 10%, 15%, 20%, 25%, or 29%). 10. In some embodiments, the improvement in OR rate is an increase of about 15%.

In some embodiments, the improvement in OR rate is statistically significant.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in an OR rate of between 56% and 90% (e.g., between 56% and 80%, between 56% and 70%, between 56% and 60%, between 60% and 90%, between 70% and 90%, between 80% and 90%, between 60% and 65%, between 65% and 70%, between 70% and 75%, between 75% and 80%, between 80% and 85%, between 85% and 90%, between 56% and 70%, between 70% and 90%, between 60% and 80%, or between 70% and 80%; e.g., about 56%, 60%, 65%, 70%, 75%, 80%, 85%, or 90%). In some embodiments, the OR rate is about 76%. In some embodiments, the OR rate is about 83%. In some embodiments, the OR rate is about 78%.

In some embodiments, the OR rate is the proportion of individuals whose best overall response is a partial response (PR) or a CR.

In one aspect, the invention features glofitamab for use in a method of treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in an improvement in CR rate of the plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.

In one aspect, the invention features glofitamab for use in a method of treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in an improvement in OR rate of the plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in CR rate of the plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in OR rate of the plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein the individual achieves a CR.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein the individual achieves an OR.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 50% (e.g., 50%±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 57% (e.g., 57%±6%, ±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 62% (e.g., 62%±6%, ±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 49% (e.g., 49%±5%, ±4%, ±3%, ±2%, or ±1%).

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 21% (e.g., 21%±2%, or ±1%), wherein the CRS event is graded according to the American Society for Transplantation and Cellular Therapy (ASTCT) consensus grading system (Lee et al., Biol Blood Marrow Transplant, 25(4): 625-638, 2019). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 23% (e.g., 23%±2%, or ±1%), wherein the CRS event is graded according to the American Society for Transplantation and Cellular Therapy (ASTCT) consensus grading system (Lee et al., Biol Blood Marrow Transplant, 25(4): 625-638, 2019). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 20% (e.g., 20%±2%, or ±1%), wherein the CRS event is graded according to the American Society for Transplantation and Cellular Therapy (ASTCT) consensus grading system (Lee et al., Biol Blood Marrow Transplant, 25(4): 625-638, 2019).

In one aspect, the invention features a method for treating relapsed or refractory DLBCL in a human individual in need thereof, comprising administering to the human individual an effective amount of glofitamab, rituximab, ifosfamide, carboplatin, and etoposide, wherein the individual does not experience a Grade 3 or higher CRS event, wherein the CRS event is graded according to the ASTCT consensus grading system.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 39.5% (e.g., 39.5%±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 45.2% (e.g., 45.2%±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 41.5% (e.g., 41.5%±4%, ±3%, ±2%, or ±1%).

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 3 or 4 adverse event (AE) event rate of about 60.5% (e.g., 60.5%±6%, ±5%, ±4%, ±3%, ±2%, or 1%).

In some embodiments, the treatment comprises administering glofitamab, rituximab, ifosfamide, carboplatin, and etoposide according to a dosing regimen comprising a first and a second dosing cycle, wherein: the first dosing cycle comprises a first dose (C1D1) of about 2.5 mg (e.g., 2.5 mg±0.01 mg, ±0.02 mg, ±0.03 mg, ±0.05 mg, ±0.1 mg, ±0.2 mg, or ±0.25 mg) of the glofitamab and a second dose (C1D2) of about 10 mg (e.g., 10 mg±0.05 mg, ±0.1 mg, ±0.2 mg, ±0.3 mg, ±0.5 mg, ±0.75 mg, or ±1 mg) of the glofitamab, and the second dosing cycle comprises a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of the glofitamab.

In some embodiments, the C1D1 and the C1D2 of the glofitamab are administered to the individual on Days 8 and 15, respectively, of the first dosing cycle. In some embodiments, the C2D1 of the glofitamab is administered to the individual on Day 8 of the second dosing cycle.

In some embodiments, the first dosing cycle comprises a single dose (C1D1) of obinutuzumab. In some embodiments, the C1D1 of obinutuzumab is about 1000 mg (e.g., 1000 mg±5 mg, ±10 mg, ±20 mg, ±30 mg, ±50 mg, ±75 mg, or ±100 mg).

In some embodiments, the C1D1 of obinutuzumab is administered on Day 1 of the first dosing cycle.

In some embodiments, dosing cycle comprises a single dose (C2D1) of rituximab. In some embodiments, the C2D1 of rituximab is about 375 mg/m² (e.g., 375 mg/m²±5 mg/m², ±10 mg/m², ±25 mg/m², or ±37.5 mg/m²). In some embodiments, the C2D1 of rituximab is administered on Day 1 of the second dosing cycle.

In some embodiments, the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and wherein the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.

In some embodiments, ifosfamide is administered at a dose of about 5000 mg/m² (e.g., 5000 mg/m²±50 mg/m², ±100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², ±200 mg/m², ±300 mg/m², or ±400 mg/m²), carboplatin is administered at a dose in mg to target area under the curve (AUC) of about 5 mg/mL/min (e.g., 5 mg/mL/min±0.05 mg/mL/min, ±0.1 mg/mL/min, 0.25 mg/mL/min, or ±0.5 mg/mL/min) with maximum dose of about 800 mg (e.g., 800 mg±10 mg, ±25 mg, ±50 mg, or ±80 mg), and etoposide is administered at a dose of about 100 mg/m² (e.g., 100 mg/m²±1 mg/m², ±2.5 mg/m², ±5 mg/m², or ±10 mg/m²) for each dose of etoposide. In some embodiments, the maximum dose of carboplatin is about 750 mg (e.g., 750 mg±10 mg, ±25 mg, ±50 mg, or ±75 mg).

In some embodiments, ifosfamide and carboplatin are administered on Day 2 of the first and second dosing cycles and the C1D1-C1D3 and C2D1-C2D3 of etoposide are administered on Days 1, 2, and 3, respectively, of each of the first and second dosing cycles.

In some embodiments, the first and second dosing cycles are each 21-day dosing cycles.

In some embodiments, the dosing regimen comprises one or more additional dosing cycles (e.g., 1, 2, 3, or 4 additional dosing cycles; e.g., 2, 3, 4, or 5 total dosing cycles).

In some embodiments, the one or more additional dosing cycles are each 21-day dosing cycles.

In some embodiments, the dosing regimen comprises three dosing cycles in total.

In some embodiments, the dosing regimen comprises four or five dosing cycles in total.

In some embodiments, the one or more additional dosing cycles comprises a third dosing cycle, and the third dosing cycle comprises:
(a) an additional single dose of glofitamab;
(b) an additional single dose of rituximab; and
(c) an additional single dose of ifosfamide; an additional single dose of carboplatin; and an additional first dose, an additional second dose, and an additional third dose of etoposide.

In some embodiments, the additional single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In some embodiments, the additional single dose of glofitamab is administered to the individual on Day 8 of the third dosing cycle.

In some embodiments, the additional single dose of rituximab is about 375 mg/m² (e.g., 375 mg/m²±5 mg/m², ±10 mg/m², +25 mg/m², or ±37.5 mg/m²).

In some embodiments, the additional single dose of rituximab is administered on Day 1 of each of the third dosing cycle.

In some embodiments, ifosfamide is administered at a dose of about 5000 mg/m² (e.g., 5000 mg/m²±50 mg/m², +100 mg/m², ±200 mg/m², ±300 mg/m², ±400 mg/m², or ±500 mg/m²), carboplatin is administered at a dose in mg to target area under the curve (AUC) of about 5 mg/mL/min (e.g., 5 mg/mL/min±0.05 mg/mL/min, 0.1 mg/mL/min, ±0.25 mg/mL/min, or ±0.5 mg/mL/min) with maximum dose of about 800 mg (e.g., 800 mg±10 mg, ±25 mg, ±50 mg, or ±80 mg), and etoposide is administered at a dose of about 100 mg/m² (e.g., 100 mg/m²±1 mg/m², ±2.5 mg/m², ±5 mg/m², or ±10 mg/m²) for each dose of etoposide. In some embodiments, the maximum dose of carboplatin is about 750 mg (e.g., 750 mg±10 mg, ±25 mg, ±50 mg, or ±75 mg).

In some embodiments, ifosfamide and carboplatin are administered on Day 2 of each of the one or more additional dosing cycles and the additional first dose, the additional second dose, and the additional third dose of etoposide are administered on Days 1, 2, and 3, respectively, of the third dosing cycle.

In some embodiments, the one or more additional dosing cycles comprises a fourth dosing cycle or a fourth and a fifth dosing cycle, and the fourth or fifth dosing cycle each comprises an additional single dose of glofitamab. In some embodiments, the single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In some embodiments, the single dose of glofitamab is administered on Day 1 of the fourth or fifth dosing cycle.

In some embodiments, the control treatment comprises administering a single dose of rituximab, a single dose of ifosfamide, a single dose of carboplatin, and a first dose, a second dose, and a third dose of etoposide according to a dosing regimen comprising three 21-day dosing cycles. In some embodiments, is administered at a dose of about 375 mg/m² (e.g., 375 mg/m²±5 mg/m², ±10 mg/m², ±25 mg/m², or ±37.5 mg/m²), ifosfamide is administered at a dose of about 5000 mg/m² (e.g., 5000 mg/m²±50 mg/m², ±100 mg/m², ±200 mg/m², +300 mg/m², ±400 mg/m², or ±500 mg/m²), carboplatin is administered at a dose in mg to target area under the curve (AUC) of about 5 mg/mL/min (e.g., 5 mg/mL/min±0.05 mg/mL/min, 0.1 mg/mL/min, ±0.25 mg/mL/min, or ±0.5 mg/mL/min) with maximum dose of about 800 mg (e.g., 800 mg±10 mg, ±25 mg, ±50 mg, or ±80 mg), and etoposide is administered at a dose of about 100 mg/m² (e.g., 100 mg/m²±1 mg/m², ±2.5 mg/m², ±5 mg/m², or ±10 mg/m²) for each dose of etoposide. In some embodiments, rituximab is administered on Day 1 of each dosing cycle, ifosfamide and carboplatin are administered on Day 2 of each of dosing cycle, and the first dose, the second dose, and the third dose of etoposide are administered on Days 1, 2, and 3, respectively, of each dosing cycle. In some embodiments, the maximum dose of carboplatin is about 750 mg (e.g., 750 mg±10 mg, ±25 mg, ±50 mg, or ±75 mg).

In some embodiments, the individual has received one prior systemic therapy.

In some embodiments, the individual is transplant or CAR-T cell therapy eligible.

In some embodiments, the treatment comprising glofitamab, rituximab, ifosfamide, carboplatin, and etoposide is administered as salvage treatment prior to CAR-T cell therapy.

In some embodiments, the DLBCL is not otherwise specified (NOS) or high-grade B-cell lymphoma (HGBCL). In some embodiments, is HGBCL with MYC and BCL-2/6 rearrangements. In some embodiments, is HGBCL NOS.

In some embodiments, the individual has extranodal disease.

In some embodiments, the method further comprises administering to the individual one or more additional therapeutic agents.

In some embodiments, the one or more additional therapeutic agent is tocilizumab. In some embodiments, tocilizumab is administered intravenously at a dose of about 8 mg/kg (e.g., 8 mg/kg±0.05 mg/kg, ±0.1 mg/kg, ±0.25 mg/kg, ±0.5 mg/kg, or ±0.8 mg/kg), not exceeding 800 mg (e.g., 800 mg±10 mg, ±25 mg, ±50 mg, or ±80 mg).

In some embodiments, the one or more additional therapeutic agents is a corticosteroid. In some embodiments, the corticosteroid comprises prednisone, prednisolone, methylprednisolone, or dexamethasone. In some embodiments, the corticosteroid is dexamethasone. In some embodiments, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the glofitamab or the obinutuzumab. In some embodiments, the corticosteroid is methylprednisolone. In some embodiments, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the glofitamab or the obinutuzumab. In some embodiments, the corticosteroid is prednisone or prednisolone. In some embodiments, prednisone or prednisolone is administered orally or intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) to the administration of any dose of the glofitamab or the obinutuzumab.

In some embodiments, the one or more additional therapeutic agents is an antihistamine. In some embodiments, the antihistamine is diphenhydramine. In some embodiments, diphenhydramine is administered orally or intravenously at a dose of about 50 mg (e.g., 50 mg±0.5 mg, ±1 mg, ±1.5 mg, +2 mg, ±3 mg, ±4 mg, or ±5 mg). In some embodiments, diphenhydramine is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the glofitamab or the obinutuzumab.

In some embodiments, the one or more additional therapeutic agents comprises granulocyte-colony stimulating factor (G-CSF). In some embodiments, G-CSF is administered between about one day and about two days after administration of any dose of rituximab, ifosfamide, carboplatin, and/or etoposide. In some embodiments, G-CSF is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day), or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In some embodiments, G-CSF is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In some embodiments, the one or more additional therapeutic agents is an antipyretic. In some embodiments, the antipyretic is acetaminophen or paracetamol. In some embodiments, acetaminophen or paracetamol is administered orally or intravenously at a dose of between about 500 to about 1000 mg (e.g., 500, 550, 600, 650, 700, 750, 800, 850, 900, 950, or 1000 mg). In some embodiments, acetaminophen or paracetamol is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the glofitamab or the obinutuzumab.

In some embodiments, the one or more additional therapeutic agents is mesna.

In some embodiments, mesna is administered intravenously at a dose of about 5000 mg/m² (e.g., 5000 mg/m²±50 mg/m², ±100 mg/m², ±200 mg/m², +300 mg/m², ±400 mg/m², or ±500 mg/m²). In some embodiments, mesna is administered via continuous infusion over about 24 hours on Day 3 of the first dosing cycle, on Day 6 of the second dosing cycle, and/or on Day 6 of each additional dosing cycle.

In some embodiments, the first dosing cycle comprises a first dose (C1D1) of ifosfamide, a second dose (C1D2) of ifosfamide, and a third dose (C1D3) of ifosfamide; a first dose (C1D1) of mesna, a second dose (C1D2) of mesna, and a third dose (C1D3) of mesna; a single dose (C1D1) of carboplatin; and a first dose (C1D1) of etoposide, a second dose (C1D2) of etoposide, and a third dose (C1D3) of etoposide; and wherein the second cycle comprises a first dose (C2D1) of ifosfamide, a second dose (C2D2) of ifosfamide, and a third dose (C2D3) of ifosfamide; a first dose (C2D1) of mesna, a second dose (C2D2) of mesna, and a third dose (C2D3) of mesna; a single dose (C2D1) of carboplatin; and a first dose (C2D1) of etoposide, a second dose (C2D2) of etoposide, and a third dose (C2D3) of etoposide.

In some embodiments, ifosfamide is administered at a dose of about 1666 mg/m² (e.g., 1666 mg/m²±25 mg/m², ±50 mg/m², +100 mg/m², or ±166.6 mg/m²) for each dose of ifosfamide, mesna is administered at a dose of about 1666 mg/m² (e.g., 1666 mg/m²±25 mg/m², ±50 mg/m², ±100 mg/m², or ±166.6 mg/m²) for each dose of mesna, carboplatin is administered at a dose in mg to target area under the curve (AUC) of about 5 mg/mL/min (e.g., 5 mg/mL/min±0.05 mg/mL/min, 0.1 mg/mL/min, 0.25 mg/mL/min, or +0.5 mg/mL/min) with maximum dose of about 800 mg (e.g., 800 mg±10 mg, ±25 mg, +50 mg, or ±80 mg), and etoposide is administered at a dose of about 100 mg/m² for each dose of etoposide (e.g., 100 mg/m²±1 mg/m², ±2.5 mg/m², ±5 mg/m², or +10 mg/m²). In some embodiments, the maximum dose of carboplatin is about 750 mg (e.g., 750 mg±10 mg, ±25 mg, ±50 mg, or ±75 mg).

In some embodiments, carboplatin is administered on Day 2 of the first and second dosing cycles and the C1D1-C1D3 and C2D1-C2D3 of ifosfamide, mesna, and etoposide are administered on Days 1, 2, and 3, respectively, of each of the first and second dosing cycles.

In some embodiments, the first and second dosing cycles are each 21-day dosing cycles. In some embodiments, the dosing regimen comprises one or more additional dosing cycles. In some embodiments, the one or more additional dosing cycles are each 21-day dosing cycles.

In some embodiments, the dosing regimen comprises three dosing cycles in total.

In some embodiments, the one or more additional dosing cycles each comprises:
(a) an additional single dose of glofitamab;
(b) an additional single dose of rituximab; and
(c) an additional first dose, an additional second dose, and an additional third dose of ifosfamide; an additional first dose, an additional second dose, and an additional third dose of mesna; an additional single dose of carboplatin; and an additional first dose, an additional second dose, and an additional third dose of etoposide.

In some embodiments, the additional single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In some embodiments, the additional single dose of glofitamab is administered to the individual on Day 8 of each of the one or more additional dosing cycles.

In some embodiments, the additional single dose of rituximab is about 375 mg/m². In some embodiments, the additional single dose of rituximab is administered on Day 1 of each of the one or more additional dosing cycles.

In some embodiments, ifosfamide is administered at a dose of about 1666 mg/m² (e.g., 1666 mg/m²±25 mg/m², ±50 mg/m², ±100 mg/m², or ±166.6 mg/m²) for each dose of ifosfamide, mesna is administered at a dose of about 1666 mg/m² (e.g., 1666 mg/m²±25 mg/m², ±50 mg/m², ±100 mg/m², or ±166.6 mg/m²) for each dose of mesna, carboplatin is administered at a dose in mg to target area under the curve (AUC) of about 5 mg/mL/min (e.g., 5 mg/mL/min±0.05 mg/mL/min, 0.1 mg/mL/min, 0.25 mg/mL/min, or ±0.5 mg/mL/min) with maximum dose of about 750 mg (e.g., 750 mg±10 mg, ±25 mg, ±50 mg, or ±75 mg), and etoposide is administered at a dose of about 100 mg/m² for each dose of etoposide (e.g., 100 mg/m²±1 mg/m², ±2.5 mg/m², ±5 mg/m², or ±10 mg/m²).

In some embodiments, carboplatin is administered on Day 2 of each of the one or more additional dosing cycles and the additional first dose, the additional second dose, and the additional third dose of ifosfamide, mesna, and etoposide are administered on Days 1, 2, and 3, respectively, of each of the one or more additional dosing cycles.

In some embodiments, rituximab, ifosfamide, mesna, carboplatin, and etoposide are administered in an outpatient setting. In some embodiments, each dose of mesna is administered simultaneously with the corresponding dose of ifosfamide on any day of a dosing cycle comprising a dose of mesna and a dose of ifosfamide.

In some embodiments, the individual is aged 18 years or older.

In some embodiments, glofitamab, rituximab, ifosfamide, mesna, carboplatin, and etoposide are administered to the induvial as salvage treatment prior to autologous stem cell transplant (ASCT) or CAR-T cell therapy.

In one embodiment, the invention features glofitamab for use in treating relapsed diffuse large B-cell lymphoma (DLBCL) in a patient eligible for autologous stem cell transplant or CAR-T cell therapy according to any of the embodiments above, wherein the patient has relapsed more than 12 months after receiving prior therapy, comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle.

A further aspect of the present invention relates to the invention as described herein.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIG. 1 is a schematic showing the overview of the study design as described in Example 1. ASCT=autologous stem cell transplantation; CAR-T=Chimeric Antigen Receptor T-cells; EOT=end of treatment; Glofit-R-ICE=Glofitamab in combination with Rituximab plus Ifosfamide, Carboplatin, Etoposide; Gpt=Obinutuzumab (GAZYVA®) pretreatment; PD=progressive disease. ^{a}: 2-3 cycles of glofit-R-ICE depending on institutional standard. ^{b}: Patients that require additional therapy between glofit-R-ICE and ASCT or CAR-T for reasons such as delayed access to definitive treatment may receive up to 2 cycles of glofitamab monotherapy if they meet specific conditions. ^{c}: ASCT conditioning regimen and supportive care per institutional standard.

FIG. 2 is a schematic showing the dosing schema of the study as described in Example 1. D=Day. ^{a}: Carboplatin dose in mg to target AUC of 5 mg/mL/min. ^{b}: Alternative dosing available for outpatient dosing: Ifosfamide 1666 mg/m² mixed with mesna 1666 mg/m² IV daily on Day 1, 2, and 3 of each cycle is permitted if R-ICE is administered in the outpatient setting.

FIG. 3 is a graph summarizing CRS events experienced by patients described in Example 4 by dosing cycle (C) and ASTCT Grade. ^{‡}No CRS events were reported after C3.

FIG. 4 is a graph summarizing the response rates in patients with primary refractory or relapsed DLBCL described in Example 4. * At the time of study entry, patients were primary refractory to or ad relapse after prior therapy. ORR: Objective response rate, PR: Partial Response, CR: Complete response.

### DETAILED DESCRIPTION OF THE INVENTION

The invention provides methods for treating an individual having a diffuse-large B-cell lymphoma (DLBCL) (e.g., a relapsed and/or refractory DLBCL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS)) by administering to the individual a treatment comprising an anti-CD20/anti-CD3 bispecific antibody (i.e., glofitamab) in combination with an anti-CD20 antibody (i.e., rituximab) and chemotherapeutic agents (i.e., ifosfamide, carboplatin and etoposide), i.e., glofit-R-ICE. In some instances, the methods further include administration of obinutuzumab.

Combining an anti-CD20/anti-CD3 bispecific antibody (i.e., glofitamab) with one or more anti-CD20 antibodies (e.g., obinutuzumab and/or rituximab) and chemotherapeutic agents (i.e., ifosfamide, carboplatin and etoposide), i.e., glofit-R-ICE, provides improved response rates to salvage chemoimmunotherapy which eventually translates into a higher percentage of patients receiving definitive therapy with ASCT or CAR-T therapy and improved survival in this treatment setting. The data provided here surprisingly shows that glofitamab in combination with R-ICE demonstrated high response rates in patients with R/R DLBCL who were eligible for ASCT or CAR T-cell therapy. High responses were observed in patients with primary refractory or relapsed disease, while having a manageable safety profile.

### (i) General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### (ii) Definitions

Terms are used herein as generally used in the art, unless otherwise defined in the following.

The term “cluster of differentiation 20” or “CD20” as used herein, refers to any native CD20 from any vertebrate source, including mammals such as primates (e.g., humans) and rodents (e.g., mice and rats), unless otherwise indicated. CD20 (also known as B-lymphocyte antigen CD20, B-lymphocyte surface antigen B1, Leu-16, Bp35, BM5, and LF5; the human protein is characterized in UniProt database entry P11836) is a hydrophobic transmembrane protein with a molecular weight of approximately 35 kD expressed on pre-B and mature B lymphocytes (Valentine, M. A. et al., J. Biol. Chem. 264 (1989) 11282-11287; Tedder, T. F., et al., Proc. Natl. Acad. Sci. U.S.A. 85 (1988) 208-212; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. The term encompasses “full-length,” unprocessed CD20 as well as any form of CD20 that results from processing in the cell. The term also encompasses naturally occurring variants of CD20, e.g., splice variants or allelic variants. Alternative splicing of this gene results in two transcript variants which encode the same protein. In one embodiment, CD20 is human CD20.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, <1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species.

By “Type II anti-CD20 antibody” is meant an anti-CD20 antibody having binding properties and biological activities of Type II anti-CD20 antibodies as described in Cragg et al., Blood 103 (2004) 2738-2743; Cragg et al., Blood 101 (2003) 1045-1052, Klein et al., mAbs 5 (2013), 22-33, and summarized in Table 1 below.

TABLE 1
Properties of type I and type II anti-CD20 antibodies

| type I anti-CD20 antibodies | type II anti-CD20 antibodies |
|---|---|
| Bind class I CD20 epitope | Bind class II CD20 epitope |
| Localize CD20 to lipid rafts | Do not localize CD20 to lipid rafts |
| High CDC * | Low CDC * |
| ADCC activity * | ADCC activity * |
| Full binding capacity to B cells | Approx. half binding capacity to B cells |
| Weak homotypic aggregation | Homotypic aggregation |
| Low cell death induction | Strong cell death induction |

* if IgG₁ isotype

Examples of type 11 anti-CD20 antibodies include, e.g., obinutuzumab (GA101), tositumumab (B1), humanized B-Ly1 antibody IgG1 (a chimeric humanized IgG1 antibody as disclosed in WO 2005/044859), 11B8 IgG1 (as disclosed in WO 2004/035607) and AT80 IgG1. In particular embodiments, the type II anti-CD20 antibody used in the methods described herein is obinutuzumab.

Examples of type I anti-CD20 antibodies include, e.g., rituximab, ofatumumab, veltuzumab, ocaratuzumab, ocrelizumab, PRO131921, ublituximab, H147 IgG3 (ECACC, hybridoma), 2C6 IgG1 (as disclosed in WO 2005/103081), 2F2 IgG1 (as disclosed in WO 2004/035607 and WO 2005/103081) and 2H7 IgG1 (as disclosed in WO 2004/056312). In particular embodiments, the type I anti-CD20 antibody used in the methods described herein is rituximab.

“CD3” refers to any native CD3 from any vertebrate source, including mammals such as primates (e.g., humans), non-human primates (e.g., cynomolgus monkeys) and rodents (e.g., mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3E). The amino acid sequence of human CD3E is shown in UniProt (www.uniprot.org) accession no. P07766 (version 144), or NCBI (www.ncbi.nlm.nih.gov/) RefSeq NP_000724.1. The amino acid sequence of cynomolgus monkey [Macaca fascicularis] CD3E is shown in NCBI GenBank no. BAB71849.1.

The terms “anti-CD20/anti-CD3 bispecific antibody” and “a bispecific antibody that binds to CD20 and CD3” refer to a bispecific antibody that is capable of binding both CD20 and CD3 with sufficient affinity such that the antibody is useful as a diagnostic and/or therapeutic agent in targeting CD20 and/or CD3. In one embodiment, the extent of binding of a bispecific antibody that binds to CD20 and CD3 to an unrelated, non-CD3 protein and/or non-CD20 protein is less than about 10% of the binding of the antibody to CD3 and/or CD20 as measured, e.g., by a radioimmunoassay (RIA). In certain embodiments, a bispecific antibody that binds to CD20 and CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, a bispecific antibody that binds to CD20 and CD3 binds to an epitope of CD3 that is conserved among CD3 from different species and/or an epitope of CD20 that is conserved among CD20 from different species. One example of an anti-CD20/anti-CD3 bispecific antibody is glofitamab (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, RO7082859, or RG6026; CAS #: 2229047-91-8).

As used herein, the term “release of cytokines” or “cytokine release” is synonymous with “cytokine storm” or “cytokine release syndrome” (abbreviated as “CRS”), and refers to an increase in the levels of cytokines, particularly tumor necrosis factor alpha (TNF-α), interferon gamma (IFN-γ), interleukin-6 (IL-6), interleukin-10 (IL-10), interleukin-2 (IL-2) and/or interleukin-8 (IL-8), in the blood of an individual during or shortly after (e.g., within 1 day of) administration of a therapeutic agent, resulting in adverse symptoms. Cytokine release is defined as a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al. 2019). In some instances, e.g., after the administration of CAR-T cells, CRS can also occur several days after administration upon expansion of the CAR-T cells. The incidence and severity typically decrease with subsequent infusions. Symptoms may range from symptomatic discomfort to fatal events, and may include fever, chills, dizziness, hypertension, hypotension, dyspnea, restlessness, sweating, flushing, skin rash, tachycardia, tachypnea, headache, tumor pain, nausea, vomiting and/or organ failure.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor. Amino acid sequence deletions and insertions include amino- and/or carboxy-terminal deletions and insertions of amino acids. Particular amino acid mutations are amino acid substitutions. For the purpose of altering, e.g., the binding characteristics of an Fc region, non-conservative amino acid substitutions, i.e., replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc region to glycine can be indicated as 329G, G329, G₃₂₉, P329G, or Pro329Gly.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., receptor and a ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “antigen binding moiety” refers to a polypeptide molecule that specifically binds to an antigenic determinant. In one embodiment, an antigen binding moiety is able to direct the entity to which it is attached (e.g., a cytokine or a second antigen binding moiety) to a target site, for example to a specific type of tumor cell or tumor stroma bearing the antigenic determinant. Antigen binding moieties include antibodies and fragments thereof as further defined herein. Preferred antigen binding moieties include an antigen binding domain of an antibody, comprising an antibody heavy chain variable region and an antibody light chain variable region. In certain embodiments, the antigen binding moieties may include antibody constant regions as further defined herein and known in the art. Useful heavy chain constant regions include any of the five isotypes: α, δ, ε, γ, or μ. Useful light chain constant regions include any of the two isotypes: κ and λ.

By “binds,” “specifically binds,” or is “specific for” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g., surface plasmon resonance technique (analyzed on a BIAcore instrument) (Liljeblad et al., Glyco J. 17, 323-329 (2000)), and traditional binding assays (Heeley, Endocr Res. 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

“Reduced binding,” for example reduced binding to an Fc receptor, refers to a decrease in affinity for the respective interaction, as measured for example by SPR. For clarity the term includes also reduction of the affinity to zero (or below the detection limit of the analytic method), i.e., complete abolishment of the interaction. Conversely, “increased binding” refers to an increase in binding affinity for the respective interaction.

As used herein, the term “antigen binding molecule” refers in its broadest sense to a molecule that specifically binds an antigenic determinant. Examples of antigen binding molecules are immunoglobulins and derivatives, e.g., fragments, thereof.

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, free in blood serum, and/or in the extracellular matrix (ECM). The proteins referred to as antigens herein (e.g., CD3) can be any native form the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. An exemplary human protein useful as antigen is CD3, particularly the epsilon subunit of CD3 (see UniProt no. P07766 (version 130), NCBI RefSeq no. NP_000724.1, for the human sequence; or UniProt no. Q95LI5 (version 49), NCBI GenBank no. BAB71849.1, for the cynomolgus [Macaca fascicularis] sequence). In certain embodiments a T cell activating bispecific antigen binding molecule described herein binds to an epitope of CD3 or a target cell antigen that is conserved among the CD3 or target cell antigen from different species.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen binding activity.

The terms “full length antibody,” “intact antibody,” and “whole antibody” are used herein interchangeably to refer to an antibody having a structure substantially similar to a native antibody structure or having heavy chains that contain an Fc region as defined herein.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen binding specificity.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, EU numbering). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e., the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the terms “first,” “second,” “third,” etc. with respect to antigen binding moieties or domains, are used for convenience of distinguishing when there is more than one of each type of moiety or domain. Use of these terms is not intended to confer a specific order or orientation unless explicitly so stated.

The terms “multispecific” and “bispecific” mean that the antigen binding molecule is able to specifically bind to at least two distinct antigenic determinants. Typically, a bispecific antigen binding molecule comprises two antigen binding sites, each of which is specific for a different antigenic determinant. In certain embodiments, a bispecific antigen binding molecule is capable of simultaneously binding two antigenic determinants, particularly two antigenic determinants expressed on two distinct cells.

The term “valent” or “valency” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3. In a particular embodiment the activating T cell antigen is CD3, particularly the epsilon subunit of CD3 (see UniProt no. P07766 (version 130), NCBI RefSeq no. NP_000724.1, for the human sequence; or UniProt no. Q95LI5 (version 49), NCBI GenBank no. BAB71849.1, for the cynomolgus [Macaca fascicularis] sequence).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating therapeutic agents used in the present invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is CD20, particularly human CD20 (see UniProt no. P11836).

A “B-cell antigen” as used herein refers to an antigenic determinant presented on the surface of a B lymphocyte, particularly a malignant B lymphocyte (in that case the antigen also being referred to as “malignant B-cell antigen”).

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

By “therapeutic agent” is meant an active ingredient, e.g., of a pharmaceutical composition, that is administered to an individual in an attempt to alter the natural course of a disease in the individual being treated, and can be performed either for prophylaxis or during the course of clinical pathology. An “immunotherapeutic agent” refers to a therapeutic agent that is administered to an individual in an attempt to restore or enhance the individual's immune response, e.g., to a tumor.

The term “pharmaceutical composition” refers to a preparation which is in such form as to permit the biological activity of an active ingredient contained therein to be effective, and which contains no additional components which are unacceptably toxic to an individual to which the composition would be administered.

A “pharmaceutically acceptable carrier” refers to an ingredient in a pharmaceutical composition, other than an active ingredient, which is nontoxic to an individual. A pharmaceutically acceptable carrier includes, but is not limited to, a buffer, excipient, stabilizer, or preservative.

The term “package insert” or “instructions for use” is used to refer to instructions customarily included in commercial packages of therapeutic products that contain information about the indications, usage, dosage, administration, combination therapy, contraindications and/or warnings concerning the use of such therapeutic products.

The term “combination treatment” noted herein encompasses combined administration (where two or more therapeutic agents are included in the same or separate formulations), and separate administration, in which case, administration of an antibody as reported herein can occur prior to, simultaneously, and/or following, administration of the additional therapeutic agent or agents, preferably an antibody or antibodies. In a particular embodiment, the combination treatment described herein refers to glofitamab, rituximab, ifosfamide, carboplatin, and etoposide (glofit-R-ICE). In another particular embodiment, a control combination treatment described herein refers to rituximab, ifosfamide, carboplatin, and etoposide (R-ICE), e.g., in the absence of glofitamab.

By a “crossover” Fab molecule (also termed “Crossfab”) is meant a Fab molecule wherein the variable domains or the constant domains of the Fab heavy and light chain are exchanged (i.e., replaced by each other), i.e., the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e., comprising a heavy chain composed of the heavy chain variable and constant domains (VH-CH1, in N- to C-terminal direction), and a light chain composed of the light chain variable and constant domains (VL-CL, in N- to C-terminal direction).

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

The term “about” as used herein refers to the usual error range for the respective value readily known to the skilled person in this technical field. Reference to “about” a value or parameter herein includes (and describes) embodiments that are directed to that value or parameter per se. In some instances, reference to “about” a value or parameter herein includes and describes embodiments that are directed to that value ±10%.

By “B-cell lymphoma” is meant a lymphoma wherein the number of B cells in a patient is increased as compared to the number of B cells in a healthy individual, and particularly wherein the increase in the number of B cells is the cause or hallmark of the disease. A “CD20-positive B-cell lymphoma” is a B-cell lymphoma wherein B-cells, particularly malignant B-cells (in addition to normal B-cells), express CD20. In some embodiments, a B cell proliferative disorder is a B-cell lymphoma (e.g., a non-Hodgkin's lymphoma (NHL); e.g., a relapsed or refractory (R/R) NHL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS).

Exemplary B-cell lymphomas include Non-Hodgkin lymphoma (NHL), diffuse large B-cell lymphoma (DLBCL; e.g., relapsed or refractory DLBCL not otherwise specified (NOS), high grade B-cell lymphoma (HGBCL; e.g., HGBCL NOS, double-hit HGBCL, and triple-hit HGBCL), primary mediastinal large B-cell lymphoma (PMBCL), and DLBCL arising from FL (transformed FL; trFL)); follicular lymphoma (FL), including Grades 1-3b FL; mantle-cell lymphoma (MCL); and marginal zone lymphoma (MZL), including splenic, nodal or extra-nodal MZL. In one embodiment, the CD20-positive B cell proliferative disorder is a Burkitt lymphoma (BL); a Burkitt leukemia (BAL; mature B-cell leukemia FAB L3); DLBCL, or PMBCL. In one embodiment the B-cell lymphoma is B-cell lymphoma a non-Hodgkin's lymphoma (NHL); e.g., a relapsed or refractory (R/R) NHL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS.

“Refractory disease” is defined as failure to achieve complete remission to first-line therapy, including defined as failure to achieve complete remission to first-line therapy, including:
Progressive Disease (PD) as best response to first-line therapy
Stable disease (SD) as best response after at least 4 cycles of first-line therapy (e.g., 4 cycles of R-CHOP)
Partial Response (PR) as best response after at least 6 cycles and with either biopsy-proven residual disease or subsequent disease progression.

“Relapsed disease” is defined as complete remission to first-line therapy. In one embodiment disease relapse is proven by biopsy. In one embodiment, patients have relapsed after or failed to respond to at least two prior systemic treatment regimens (including at least one prior regimen containing anthracycline, and at least one containing an anti CD20-directed therapy).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the individual or subject is a human. In one instance, each subject in a population of subjects is human. In one instance, each subject in a reference population of subjects is human. In one embodiment, an individual is considered to be a pediatric patient if the pediatric patient is younger than 18 years old (i.e., aged 17 years or less). In one embodiment, the pediatric patient is aged between 6 months and 17 years. In one embodiment, an individual is considered to be a young adult patient if the young adult patient is aged between 18 years and 30 years. In some instances, a subject is an individual in need of treatment, e.g., for a disease (e.g., a B-cell lymphoma).

A “transplant eligible” individual or an individual “eligible for autologous stem cell transplantation (SCT)” is an individual who meets eligibility for, who is recommended for or who can receive, autologous SCT. In one embodiment, “transplant eligible” is defined as being medically eligible for intensive platinum-based salvage therapy followed by autologous stem cell transplantation (ASCT). In one embodiment the transplant eligible subject achieves an objective response as well as mobilization of the target dose of at least 2,000,000 CD34+ hematopoietic stem cells/kg. In one embodiment, “transplant eligible” is defined as being medically eligible for two to three cycles of salvage therapy with R-ICE and glofitamab to achieve CR followed by allogeneic or autologous hematopoietic stem cell transplantation (HSCT).

A “CAR-T cell therapy eligible” individual or an individual “eligible for CAR-T cell therapy” is an individual who meets eligibility for, who is recommended for or who can receive, chimeric antigen receptor (CAR) T-cell therapy.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods of the invention are used to delay development of a disease or to slow the progression of a disease. In one embodiment, the disease being treated is a B-cell lymphoma (e.g., a diffuse large B cell lymphoma (DLBCL); e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS).

As used herein, “delaying progression” of a disorder or disease means to defer, hinder, slow, retard, stabilize, and/or postpone development of the disease or disorder (e.g., B-cell lymphoma (e.g., a diffuse large B cell lymphoma (DLBCL); e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS)). This delay can be of varying length of time, depending on the history of the disease and/or individual being treated. As is evident to one skilled in the art, a sufficient or significant delay can, in effect, encompass prevention, in that the individual does not develop the disease. For example, in a late-stage cancer, development of central nervous system (CNS) metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with glofitamab+rituximab, ifosfamide, carboplatin, and etoposide (R-ICE) dosing regimen of the invention relative to a reference treatment, e.g., comprising R-ICE only and without glofitamab. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the B-cell lymphoma (e.g., a diffuse large B cell lymphoma (DLBCL); e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS), the presence or size of metastases, or the size of the primary tumor. In one embodiment, the CD20-positive B cell proliferative disorder being treated is a non-Hodgkin's lymphoma (NHL); e.g., a relapsed or refractory (R/R) NHL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS.

As used herein, “administering” is meant a method of giving a dosage of a compound (e.g., glofitamab, obinutuzumab, rituximab, ifosfamide, carboplatin, or etoposide, and/or one or more additional therapeutic agents described herein) or a composition (e.g., a pharmaceutical composition, e.g., a pharmaceutical composition including glofitamab, obinutuzumab, rituximab, ifosfamide, carboplatin, or etoposide, and/or one or more additional therapeutic agents described herein) to an individual. The compounds and/or compositions utilized in the methods described herein can be administered intravenously (e.g., by intravenous infusion).

A “fixed” or “flat” dose of a therapeutic agent (e.g., a bispecific antibody) herein refers to a dose that is administered to a patient without regard for the weight or body surface area (BSA) of the patient.

The fixed or flat dose is therefore not provided as a mg/kg dose or a mg/m² dose, but rather as an absolute amount of the therapeutic agent (e.g., mg).

A “target dose” herein refers to the dose of glofitamab that achieves therapeutic effect, i.e., achieves the desired clinical efficacy. It was found that for glofitamab a possible target dose is 16 mg or 30 mg.

An “unchanging or preset dosing with target dose” and a “treatment regimen without a step-up dosing regimen” refers to a dosing schedule that uses the same dosage in the first and second cycle and optionally also any subsequent treatment cycle, as opposed to step-up dosing, which uses lower dosages in the first few treatment cycles and only reaches the target dose in the second or in a later treatment cycle.

The terms “treatment cycle” or “cycle” (abbreviated: “C”) as used herein mean a course of one or more doses of glofitamab, e.g., in combination with rituximab, ifosfamide, obinutuzumab, and etoposide, that is repeated on a regular schedule, optionally with periods of rest (no treatment) in between. In one aspect of the invention, the first treatment cycle comprises a first and a second dose of glofitamab, followed by a period of rest. In one such embodiment, the first treatment cycle comprises a first dose of glofitamab on Day 8 of the first cycle, and a second dose of glofitamab on Day 15 of the first cycle, followed by 6 days of rest. In one embodiment the second and any subsequent cycles comprise one dose of glofitamab given on Day 8 of that cycle, followed by 13 days of rest. In one embodiment, one treatment cycle comprises 21 days. In another embodiment, one treatment cycle comprises 14 days. The treatment schedule according to the invention may comprise 2 or more treatment cycles, in particular 3 treatment cycles. In some embodiments, a treatment cycle is referred to as a “dosing cycle.”

“Individual response” or “response” can be assessed using any endpoint indicating a benefit to the individual, including, without limitation, (1) inhibition, to some extent, of disease progression (e.g., progression of a B-cell lymphoma (e.g., a diffuse large B cell lymphoma (DLBCL); e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS)); including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the B-cell lymphoma; (6) increase or extend in the length of survival, including overall survival and progression-free survival; and/or (7) decreased mortality at a given point of time following treatment.

As used herein, “complete response” or “CR” refers to disappearance of all target lesions. In one embodiment standard NHL response criteria are assessed for determining CR. (Lugano Classification, Cheson et al. J Clin Oncol. 2014 Sep. 20; 32(27): 3059-3067.). In one embodiment the CR rate is defined as the proportion of participants that achieves a CR within three cycles of glofit-R-ICE, as determined by the investigator according to Lugano criteria. In one embodiment CR is defined as complete metabolic response as determined by PET/CT of the lymph nodes and extra-lymphatic sites, with a score of 1, 2, or 3 with or without a residual mass on 5PS, wherein PET 5PS: 1=no uptake above background; 2=uptake>mediastinum; 3=uptake>mediastinum but s liver; 4=uptake moderately>liver; 5=uptake markedly higher than liver and/or new lesions; X=new areas of uptake unlikely to be related to lymphoma. In one embodiment, CR is defined as complete radiologic response as determined by CT of the lymph nodes and extra-lymphatic sites, wherein the target nodes/nodal masses must regress to ≤1.5 cm in LDi (longest transverse diameter of a lesion) and no extralymphatic sites of disease remain.

“Extralymphatic” as used herein means sites in the body other than lymph nodes and other lymphatic structures. These lymphatic structures include spleen, thymus gland, Waldeyer's ring (tonsils), Peyer's patches (ileum) and lymphoid nodules in the appendix.

“Extranodal disease” in the context of the present invention refers to lymphoma that originates in or spreads to sites outside of the lymph nodes, such as the gastrointestinal tract (stomach/bowel), skin, bone, brain, testes, or other organs.

As used herein, “partial response” or “PR” refers to partial metabolic response as determined by PET/CT of the lymph nodes and extra-lymphatic sites and/or Partial remission as determined by CT of the lymph nodes and extra-lymphatic sites. In one embodiment, Partial metabolic response is defined by a score 4 or 5 b with reduced uptake compared with baseline and residual mass(es) of any size as determined by PET/CT of the lymph nodes and extra-lymphatic sites, wherein PET 5PS: 1=no uptake above background; 2=uptake>mediastinum; 3=uptake>mediastinum but s liver; 4=uptake moderately>liver; 5=uptake markedly higher than liver and/or new lesions; X=new areas of uptake unlikely to be related to lymphoma. In one embodiment partial remission is defined as at least a 50% decrease in the product of the diameters (SPD) of up to 6 target measurable nodes and extranodal sites, taking as reference the baseline SPD.

As used herein, “overall response” or “objective response” or “OR” refers any response, including a partial or a complete response. In some instances, an OR can be calculated as the sum of the PR and CR.

An “effective response” of an individual or an individual's “responsiveness” to treatment with a medicament (e.g., glofitamab+R-ICE treatment regimen) and similar wording refers to the clinical or therapeutic benefit imparted to an individual as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

“Duration of complete response” (DOCR) is defined as the time from the first occurrence of a documented complete response to disease progression or death from any cause (whichever occurs first), as determined by the investigator according to Lugano criteria (Cheson et al. J Clin Oncol. 2014 Sep. 20; 32(27): 3059-3067.).

“Duration of objective response” (DOR), defined as the time from the first occurrence of a documented objective response (CR or PR) to disease progression or death from any cause (whichever occurs first), as determined by the investigator according to Lugano criteria (Cheson et al. J Clin Oncol. 2014 Sep. 20; 32(27): 3059-3067.).

“Progression-free survival” (PFS) is defined as the time from the first treatment with glofitamab+R-ICE treatment regimen to the first occurrence of disease progression or death from any cause, whichever occurs first. In one embodiment, PFS is assessed based on the Lugano Classification (Cheson et al. J Clin Oncol. 2014 Sep. 20; 32(27): 3059-3067.).

“Overall survival” (OS) is defined as time from the first treatment with glofitamab to the date of death from any cause.

As used herein “Event-free survival” (EFS) is defined as the time from the first treatment with glofitamab+R-ICE treatment regimen to the first occurrence of disease progression as determined by the investigator according to Lugano criteria, initiation of new anti-lymphoma therapy (not including planned ASCT), or death from any cause (whichever occurs first).

As used herein, “objective response rate” (ORR) is defined as the sum of partial response (PR) rate and complete response (CR) rate. In one embodiment, ORR is evaluated based on the Lugano Classification (Cheson et al. J Clin Oncol. 2014 Sep. 20; 32(27): 3059-3067). In one embodiment, the ORR is defined as the proportion of participants that achieves a CR or PR within three cycles of glofitamab+R-ICE treatment regimen described therein. CR and/or PR is assessed by the investigator, using the International Pediatric NHL Response Criteria for pediatric individuals <18 years old (Sandlund J T, Guillerman R P, Perkins S L, et al. International pediatric non-Hodgkin lymphoma response criteria. J. Clin. Oncol. 33:2106-2111, 2015).

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, an “infusion-related reaction,” “IRR,” or infusion-related adverse event” is an adverse event that occurs in a patient or individual during or within 24 hours after administration of a drug (e.g., an anti-CD20/anti-CD3 bispecific antibody, e.g., glofitamab; or an anti-CD20 antibody, e.g., obinutuzumab or rituximab). IRRs may be graded as Grades 1-5 according to, e.g., NCI CTCAE v.4.

As used herein, “mobilization-adjusted response rate” or “MARR” refers to the percentage of patients treated with intent to proceed to autologous stem cell transplant (ASCT) therapy, who achieve an objective response (e.g., OR; e.g., PR or CR) as well as mobilization of the target dose of 2,000,000 CD34+ hematopoietic stem cells/kg typically required as a minimum for ASCT. In some instances, MARR (e.g., OR, PR, and CR) are determined according to the Lugano criteria.

As used herein, “event free survival” or “EFS” refers to the time from first study treatment to the first occurrence of disease progression, initiation of new anti-lymphoma therapy, or death from any cause. In some instances, EFS (e.g., disease progression) is determined according to the Lugano criteria.

As used herein, “duration of response” or “DOR” refers to the time from first documented CR or PR to disease progression or death from any cause. In some instances, DOR (e.g., CR, PR, and disease progression) is determined according to the Lugano criteria.

As used herein, “duration of response” or “DOR” refers to the time from first documented CR or PR to disease progression or death from any cause. In some instances, DOR (e.g., CR, PR, and disease progression) is determined according to the Lugano criteria.

As used herein, “duration of complete response” or “DOCR” refers to the time from first documented CR to disease progression or death from any cause. In some instances, DOR (e.g., CR, PR, and disease progression) is determined according to the Lugano criteria.

As used herein, the term “R-ICE” refers to Rituximab plus ifosfamide, carboplatin, etoposide or etoposide phosphate. As used herein, the term “ICE,” refers to ifosfamide, carboplatin, etoposide or etoposide phosphate.

An “improvement in CR rate” refers to an increase in the rate of CR observed for individuals of a plurality who receive a study treatment (e.g., glofit-R-ICE) as compared to the rate of CR observed for individuals of a plurality who receive a control treatment (e.g., R-ICE alone, without glofitamab).

An “improvement in OR rate” refers to an increase in the rate of OR observed for individuals of a plurality who receive a study treatment (e.g., glofit-R-ICE) as compared to the rate of OR observed for individuals of a plurality who receive a control treatment (e.g., R-ICE alone, without glofitamab).

An “improvement in median EFS” refers to an increase in the median EFS observed for individuals of a plurality who receive a study treatment (e.g., glofit-R-ICE) as compared to the median EFS observed for individuals of a plurality who receive a control treatment (e.g., R-ICE alone, without glofitamab).

An “improvement in MARR” refers to an increase in the MARR observed for individuals of a plurality who receive a study treatment (e.g., glofit-R-ICE) as compared to the MARR observed for individuals of a plurality who receive a control treatment (e.g., R-ICE alone, without glofitamab).

An “improvement in median DOR” refers to an increase in the median DOR observed for individuals of a plurality who receive a study treatment (e.g., glofit-R-ICE) as compared to median DOR observed for individuals of a plurality who receive a control treatment (e.g., R-ICE alone, without glofitamab).

An “improvement in median DOCR” refers to an increase in the median DOCR observed for individuals of a plurality who receive a study treatment (e.g., glofit-R-ICE) as compared to median DOCR observed for individuals of a plurality who receive a control treatment (e.g., R-ICE alone, without glofitamab).

An “improvement in median OS” refers to an increase in the median OS observed for individuals of a plurality who receive a study treatment (e.g., glofit-R-ICE) as compared to median OS observed for individuals of a plurality who receive a control treatment (e.g., R-ICE alone, without glofitamab).

“Control treatment” refers to treatment with R-ICE alone, without glofitamab. The term also encompasses R-ICE treatments that have been conducted in separate clinical studies, i.e. not in the same clinical study as the glofit-R-ICE treatment. In some embodiments the control treatment refers to treatment with R-ICE alone in the approved setting, e.g. post-marketing surveillance or real-world use, Phase IV trials or post-market studies.

### (iii) Combination Treatment of an Anti-CD20/Anti-CD3 Bispecific Antibody with an Anti-CD20 Antibody and Chemotherapy

The invention provides methods for treating an individual having a B-cell lymphoma (e.g., a non-Hodgkin's lymphoma (NHL); e.g., a relapsed or refractory (R/R) NHL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS) that includes administering to the individual glofitamab in combination with rituximab, ifosfamide, carboplatin and etoposide.

In some instances, the present methods are used for treating an individual having a B-cell lymphoma (e.g., a non-Hodgkin's lymphoma (NHL); e.g., a relapsed or refractory (R/R) NHL; e.g., a diffuse large B-cell lymphoma (DLBCL), not otherwise specified (NOS), Epstein-Barr virus [EBV]+ DLBCL, High-grade B-cell lymphoma (HGBCL), High-grade B-cell lymphoma (HGBCL) with MYC and B-cell lymphoma 2 and/or B-cell lymphoma 6 rearrangements, or HGBCL, NOS; e.g., R/R diffuse large B-cell lymphoma (DLBCL), not otherwise specified (NOS), R/R Epstein-Barr virus [EBV]+ DLBCL, R/R High-grade B-cell lymphoma (HGBCL), R/R High-grade B-cell lymphoma (HGBCL) with MYC and B-cell lymphoma 2 and/or B-cell lymphoma 6 rearrangements, or R/R HGBCL, NOS). In a particular embodiment, the individual has relapsed or refractory DLBCL. In some embodiments, the individual has received one prior systemic therapy. In some embodiments, the individual is transplant or CAR-T cell therapy eligible. In some embodiments, the treatment comprising glofitamab, rituximab, ifosfamide, carboplatin, and etoposide is administered as salvage treatment prior to CAR-T cell therapy. In some embodiments, the DLBCL is not otherwise specified (NOS) or high-grade B-cell lymphoma (HGBCL). In some embodiments, the DLBCL is HGBCL with MYC and BCL-2/6 rearrangements. In some embodiments, the DLBCL is HGBCL NOS. In some embodiments, the individual has extranodal disease. In some embodiments, the individual is aged 18 years or older (e.g., an adult). In some embodiments, the individual is not a pediatric patient (e.g., less than 18 years of age).

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in complete response (CR) rate of a plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.

In some embodiments, the improvement in CR rate is between 8% and 44% (e.g., between 8% and 40%, between 8% and 30%, between 8% and 20%, between 10% and 44%, between 20% and 44%, between 30% and 44%, between 20% and 30%, between 10% and 40%, between 10% and 15%, between 15% and 20%, between 20% and 25%, between 25% and 30%, between 30% and 35%, or between 35% and 40%; about 8%, 10%, 15%, 20%, 25%, 30%, 35%, 40%, or 44%). In some embodiments, the improvement in CR rate is an increase of about 28%.

In some embodiments, the improvement in CR rate is statistically significant.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in a CR rate of between 45% and 83% (e.g., between 45% and 80%, between 45% and 70%, between 45% and 60%, between 45% and 50%, between 55% and 83%, between 60% and 83%, between 70% and 83%, between 50% and 55%, between 55% and 60%, between 60% and 65%, between 65% and 70%, between 75% and 80%, between 50% and 70%, between 60% and 80%, or between 60% and 70%; about 45%, 50%, 55%, 60%, 65%, 70%, 75%, 80%, or 83%). In some embodiments, the CR rate is about 66%. In some embodiments, the CR rate is about 67%. In some embodiments, the CR rate is about 69%.

In some embodiments, the CR rate is the proportion of individuals whose best overall response is a CR as determined by positron emission tomography-computed tomography (PET-CT) or positron emission tomography-magnetic resonance imaging (PET-MRI).

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in overall response (OR) rate of a plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.

In some embodiments, the improvement in OR rate is between 1% and 29% (e.g., between 1% and 20%, between 1% and 10%, between 10% and 29%, between 20% and 29%, between 1% and 5%, between 5% and 10%, between 10% and 15%, between 15% and 20%, between 20% and 25%, between 25% and 29%, between 5% and 15%, between 15% and 29%, or between 10% and 20%; about 1%, 5%, 10%, 15%, 20%, 25%, or 29%). 10. In some embodiments, the improvement in OR rate is an increase of about 15%.

In some embodiments, the improvement in OR rate is statistically significant.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in an OR rate of between 56% and 90% (e.g., between 56% and 80%, between 56% and 70%, between 56% and 60%, between 60% and 90%, between 70% and 90%, between 80% and 90%, between 60% and 65%, between 65% and 70%, between 70% and 75%, between 75% and 80%, between 80% and 85%, between 85% and 90%, between 56% and 70%, between 70% and 90%, between 60% and 80%, or between 70% and 80%; e.g., about 56%, 60%, 65%, 70%, 75%, 80%, 85%, or 90%). In some embodiments, the OR rate is about 76%. In some embodiments, the OR rate is about 83%. In some embodiments, the OR rate is about 78%.

In some embodiments, the OR rate is the proportion of individuals whose best overall response is a partial response (PR) or a CR.

In one aspect, the invention features glofitamab for use in a method of treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in an improvement in CR rate of the plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.

In one aspect, the invention features glofitamab for use in a method of treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in an improvement in OR rate of the plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in CR rate of the plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in OR rate of the plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein the individual achieves a CR.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein the individual achieves an OR.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 50% (e.g., 50%±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 57% (e.g., 57%±6%, ±5%, ±4%, ±3%, ±2%, or 1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 62% (e.g., 62%±6%, ±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 49% (e.g., 49%±5%, ±4%, ±3%, ±2%, or ±1%).

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 21% (e.g., 21%±2%, or ±1%), wherein the CRS event is graded according to the American Society for Transplantation and Cellular Therapy (ASTCT) consensus grading system (Lee et al., Biol Blood Marrow Transplant, 25(4): 625-638, 2019). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 23% (e.g., 23%±2%, or +1%), wherein the CRS event is graded according to the American Society for Transplantation and Cellular Therapy (ASTCT) consensus grading system (Lee et al., Biol Blood Marrow Transplant, 25(4): 625-638, 2019). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 20% (e.g., 20%±2%, or ±1%), wherein the CRS event is graded according to the American Society for Transplantation and Cellular Therapy (ASTCT) consensus grading system (Lee et al., Biol Blood Marrow Transplant, 25(4): 625-638, 2019).

In one aspect, the invention features a method for treating relapsed or refractory DLBCL in a human individual in need thereof, comprising administering to the human individual an effective amount of glofitamab, rituximab, ifosfamide, carboplatin, and etoposide, wherein individual does not experience a Grade 3 or higher CRS event, wherein the CRS event is graded according to the ASTCT consensus grading system.

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 39.5% (e.g., 39.5%±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 45.2% (e.g., 45.2%±5%, ±4%, ±3%, ±2%, or ±1%). In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 41.5% (e.g., 41.5%±4%, ±3%, ±2%, or ±1%).

In one aspect, the invention features a method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 3 or 4 adverse event (AE) event rate of about 60.5% (e.g., 60.5%±6%, ±5%, ±4%, ±3%, ±2%, or 1%).

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in median event free survival (EFS) of a plurality of human individuals as compared to a reference median EFS, wherein the reference median EFS is the median EFS of a plurality of human individuals who have received a control treatment comprising rituximab, ifosfamide, carboplatin, and etoposide in the absence of glofitamab.

In one aspect, the invention features glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median EFS of a plurality of human individuals as compared to a reference median EFS, wherein the reference median EFS is the median EFS of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median EFS of a plurality of human individuals as compared to a reference median EFS, wherein the reference median EFS is the median EFS of a plurality of human individuals who have received a control treatment comprising rituximab, ifosfamide, carboplatin, and etoposide in the absence of glofitamab.

In some embodiments, EFS is defined as the time from first study treatment to the first occurrence of disease progression, initiation of new anti-lymphoma therapy, or death from any cause.

In some embodiments, EFS is determined according to the Lugano criteria.

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in mobilization-adjusted response rate (MARR) of a plurality of human individuals as compared to a reference MARR, wherein the reference MARR is the MARR of a plurality of human individuals who have received a control treatment comprising rituximab, ifosfamide, carboplatin, and etoposide in the absence of glofitamab.

In one aspect, the invention features glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in MARR of a plurality of human individuals as compared to a reference MARR, wherein the reference MARR is the MARR of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in MARR of a plurality of human individuals as compared to a reference MARR, wherein the reference MARR is the MARR of a plurality of human individuals who have received a control treatment comprising rituximab, ifosfamide, carboplatin, and etoposide in the absence of glofitamab.

In some embodiments, MARR is defined as the rate of individuals treated with intent to proceed to autologous stem cell transplant (ASCT) therapy that achieve a CR or PR within three cycles of treatment, and additionally achieves mobilization of a minimum of 2,000,000 CD34+ hematopoietic stem cells/kg for ASCT.

In some embodiments, CR or PR is determined according to the Lugano criteria.

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in median duration of response (DOR) of a plurality of human individuals as compared to a reference median DOR, wherein the reference median DOR is the median DOR of a plurality of human individuals who have received a control treatment comprising rituximab, ifosfamide, carboplatin, and etoposide in the absence of glofitamab.

In one aspect, the invention features glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median DOR of a plurality of human individuals as compared to a reference median DOR, wherein the reference median DOR is the median DOR of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median DOR of a plurality of human individuals as compared to a reference median DOR, wherein the reference median DOR is the median DOR of a plurality of human individuals who have received a control treatment comprising rituximab, ifosfamide, carboplatin, and etoposide in the absence of glofitamab.

In some embodiments, DOR is defined as the time from first documented CR or PR to disease progression or death from any cause.

In some embodiments, DOR is determined according to the Lugano criteria.

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in median duration of complete response (DOCR) of a plurality of human individuals as compared to a reference median DOCR, wherein the reference median DOCR is the median DOCR of a plurality of human individuals who have received a control treatment comprising rituximab, ifosfamide, carboplatin, and etoposide in the absence of glofitamab.

In one aspect, the invention features glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median DOCR of a plurality of human individuals as compared to a reference median DOCR, wherein the reference median DOCR is the median DOCR of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median DOCR of a plurality of human individuals as compared to a reference median DOCR, wherein the reference median DOCR is the median DOCR of a plurality of human individuals who have received a control treatment comprising rituximab, ifosfamide, carboplatin, and etoposide in the absence of glofitamab.

In some embodiments, DOCR is defined as the time from first documented CR to disease progression or death from any cause.

In some embodiments, DOCR is determined according to the Lugano criteria.

In one aspect, the invention features a method for treating relapsed or refractory diffuse large B-cell lymphoma (DLBCL) in a human individual in need thereof, comprising administering to the human individual an effective amount of glofitamab, rituximab, ifosfamide, carboplatin, and etoposide, wherein administering such treatment to a plurality of human individuals results in an improvement in median overall survival (OS) of a plurality of human individuals as compared to a reference median OS, wherein the reference median OS is the median OS of a plurality of human individuals who have received a control treatment comprising rituximab, ifosfamide, carboplatin, and etoposide in the absence of glofitamab.

In one aspect, the invention features glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median OS of a plurality of human individuals as compared to a reference median OS, wherein the reference median OS is the median OS of a plurality of human individuals who have received a control treatment comprising rituximab, ifosfamide, carboplatin, and etoposide in the absence of glofitamab.

In one aspect, the invention features use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual, wherein administering such treatment to a plurality of human individuals results in an improvement in median OS of a plurality of human individuals as compared to a reference median OS, wherein the reference median OS is the median OS of a plurality of human individuals who have received a control treatment comprising rituximab, ifosfamide, carboplatin, and etoposide in the absence of glofitamab.

In some embodiments, OS is defined as the time from first study treatment to death from any cause.

In some embodiments, the treatment comprises administering glofitamab, rituximab, ifosfamide, carboplatin, and etoposide according to a dosing regimen comprising a first and a second dosing cycle, wherein:
the first dosing cycle comprises a first dose (C1D1) of about 2.5 mg (e.g., 2.5 mg±0.01 mg, ±0.02 mg, ±0.03 mg, ±0.05 mg, ±0.1 mg, ±0.2 mg, or ±0.25 mg) of the glofitamab and a second dose (C1D2) of about 10 mg (e.g., 10 mg±0.05 mg, ±0.1 mg, ±0.2 mg, ±0.3 mg, ±0.5 mg, ±0.75 mg, or ±1 mg) of the glofitamab, and the second dosing cycle comprises a single dose (C2D1) of about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg) of the glofitamab.

In some embodiments, the C1D1 and the C1D2 of the glofitamab are administered to the individual on Days 8 and 15, respectively, of the first dosing cycle. In some embodiments, the C2D1 of the glofitamab is administered to the individual on Day 8 of the second dosing cycle.

In some embodiments, the first dosing cycle comprises a single dose (C1D1) of obinutuzumab. In some embodiments, the C1D1 of obinutuzumab is about 1000 mg (e.g., 1000 mg±5 mg, ±10 mg, ±20 mg, ±30 mg, ±50 mg, ±75 mg, or ±100 mg).

In some embodiments, the C1D1 of obinutuzumab is administered on Day 1 of the first dosing cycle.

In some embodiments, dosing cycle comprises a single dose (C2D1) of rituximab. In some embodiments, the C2D1 of rituximab is about 375 mg/m² (e.g., 375 mg/m²±5 mg/m², ±10 mg/m², ±25 mg/m², or ±37.5 mg/m²). In some embodiments, the C2D1 of rituximab is administered on Day 1 of the second dosing cycle.

In some embodiments, the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and wherein the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.

In some embodiments, ifosfamide is administered at a dose of about 5000 mg/m² (e.g., 5000 mg/m²±50 mg/m², +100 mg/m², ±200 mg/m², ±300 mg/m², ±400 mg/m², or ±500 mg/m²), about 4000 mg/m² (e.g., 4000 mg/m²±40 mg/m², ±50 mg/m², ±100 mg/m², +200 mg/m², ±300 mg/m², or ±400 mg/m²), carboplatin is administered at a dose in mg to target area under the curve (AUC) of about 5 mg/mL/min (e.g., 5 mg/mL/min±0.05 mg/mL/min, ±0.1 mg/mL/min, 0.25 mg/mL/min, or ±0.5 mg/mL/min) with maximum dose of about 800 mg (e.g., 800 mg±10 mg, ±25 mg, ±50 mg, or ±80 mg), and etoposide is administered at a dose of about 100 mg/m² (e.g., 100 mg/m²±1 mg/m², ±2.5 mg/m², ±5 mg/m², or ±10 mg/m²) for each dose of etoposide. In some embodiments, the maximum dose of carboplatin is about 750 mg (e.g., 750 mg±10 mg, ±25 mg, ±50 mg, or ±75 mg).

In some embodiments, ifosfamide and carboplatin are administered on Day 2 of the first and second dosing cycles and the C1D1-C1D3 and C2D1-C2D3 of etoposide are administered on Days 1, 2, and 3, respectively, of each of the first and second dosing cycles.

In some embodiments, the first and second dosing cycles are each 21-day dosing cycles.

In some embodiments, the dosing regimen comprises one or more additional dosing cycles (e.g., 1, 2, 3, or 4 additional dosing cycles; e.g., 2, 3, 4, or 5 total dosing cycles).

In some embodiments, the one or more additional dosing cycles are each 21-day dosing cycles.

In some embodiments, the dosing regimen comprises three dosing cycles in total.

In some embodiments, the dosing regimen comprises four or five dosing cycles in total.

In some embodiments, the one or more additional dosing cycles comprises a third dosing cycle, and the third dosing cycle comprises:
(a) an additional single dose of glofitamab;
(b) an additional single dose of rituximab; and
(c) an additional single dose of ifosfamide; an additional single dose of carboplatin; and an additional first dose, an additional second dose, and an additional third dose of etoposide.

In some embodiments, the additional single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg).

In some embodiments, the additional single dose of glofitamab is administered to the individual on Day 8 of the third dosing cycle.

In some embodiments, the additional single dose of rituximab is about 375 mg/m² (e.g., 375 mg/m²±5 mg/m², ±10 mg/m², +25 mg/m², or ±37.5 mg/m²).

In some embodiments, the additional single dose of rituximab is administered on Day 1 of each of the third dosing cycle.

In some embodiments, ifosfamide is administered at a dose of about 5000 mg/m² (e.g., 5000 mg/m²±50 mg/m², +100 mg/m², ±200 mg/m², ±300 mg/m², ±400 mg/m², or ±500 mg/m²), carboplatin is administered at a dose in mg to target area under the curve (AUC) of about 5 mg/mL/min (e.g., 5 mg/mL/min±0.05 mg/mL/min, 0.1 mg/mL/min, ±0.25 mg/mL/min, or ±0.5 mg/mL/min) with maximum dose of about 800 mg (e.g., 800 mg±10 mg, ±25 mg, ±50 mg, or ±80 mg), and etoposide is administered at a dose of about 100 mg/m² (e.g., 100 mg/m²±1 mg/m², ±2.5 mg/m², ±5 mg/m², or ±10 mg/m²) for each dose of etoposide. In some embodiments, the maximum dose of carboplatin is about 750 mg (e.g., 750 mg±10 mg, ±25 mg, ±50 mg, or ±75 mg).

In some embodiments, ifosfamide and carboplatin are administered on Day 2 of each of the one or more additional dosing cycles and the additional first dose, the additional second dose, and the additional third dose of etoposide are administered on Days 1, 2, and 3, respectively, of the third dosing cycle.

In some embodiments, the one or more additional dosing cycles comprises a fourth dosing cycle or a fourth and a fifth dosing cycle, and the fourth or fifth dosing cycle each comprises an additional single dose of glofitamab. In some embodiments, the single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In some embodiments, the single dose of glofitamab is administered on Day 1 of the fourth or fifth dosing cycle.

In some embodiments, the control treatment comprises administering a single dose of rituximab, a single dose of ifosfamide, a single dose of carboplatin, and a first dose, a second dose, and a third dose of etoposide according to a dosing regimen comprising three 21-day dosing cycles. In some embodiments, is administered at a dose of about 375 mg/m² (e.g., 375 mg/m²±5 mg/m², ±10 mg/m², ±25 mg/m², or ±37.5 mg/m²), ifosfamide is administered at a dose of about 5000 mg/m² (e.g., 5000 mg/m²±50 mg/m², ±100 mg/m², ±200 mg/m², +300 mg/m², ±400 mg/m², or ±500 mg/m²), carboplatin is administered at a dose in mg to target area under the curve (AUC) of about 5 mg/mL/min (e.g., 5 mg/mL/min±0.05 mg/mL/min, 0.1 mg/mL/min, ±0.25 mg/mL/min, or ±0.5 mg/mL/min) with maximum dose of about 800 mg (e.g., 800 mg±10 mg, ±25 mg, ±50 mg, or ±80 mg), and etoposide is administered at a dose of about 100 mg/m² (e.g., 100 mg/m²±1 mg/m², ±2.5 mg/m², ±5 mg/m², or ±10 mg/m²) for each dose of etoposide. In some embodiments, rituximab is administered on Day 1 of each dosing cycle, ifosfamide and carboplatin are administered on Day 2 of each of dosing cycle, and the first dose, the second dose, and the third dose of etoposide are administered on Days 1, 2, and 3, respectively, of each dosing cycle. In some embodiments, the maximum dose of carboplatin is about 750 mg (e.g., 750 mg±10 mg, ±25 mg, ±50 mg, or ±75 mg).

In some embodiments, the method further comprises administering to the individual one or more additional therapeutic agents.

In some embodiments, the one or more additional therapeutic agent is tocilizumab. In some embodiments, tocilizumab is administered intravenously at a dose of about 8 mg/kg (e.g., 8 mg/kg±0.05 mg/kg, ±0.1 mg/kg, ±0.25 mg/kg, ±0.5 mg/kg, or ±0.8 mg/kg), not exceeding 800 mg (e.g., 800 mg±10 mg, ±25 mg, ±50 mg, or ±80 mg).

In some embodiments, the one or more additional therapeutic agents is a corticosteroid. In some embodiments, the corticosteroid comprises prednisone, prednisolone, methylprednisolone, or dexamethasone. In some embodiments, the corticosteroid is dexamethasone. In some embodiments, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the glofitamab or the obinutuzumab. In some embodiments, the corticosteroid is methylprednisolone. In some embodiments, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the glofitamab or the obinutuzumab. In some embodiments, the corticosteroid is prednisone or prednisolone. In some embodiments, prednisone or prednisolone is administered orally or intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) to the administration of any dose of the glofitamab or the obinutuzumab.

In some embodiments, the one or more additional therapeutic agents is an antihistamine. In some embodiments, the antihistamine is diphenhydramine. In some embodiments, diphenhydramine is administered orally or intravenously at a dose of about 50 mg (e.g., 50 mg±0.5 mg, ±1 mg, ±1.5 mg, ±2 mg, ±3 mg, ±4 mg, or ±5 mg). In some embodiments, diphenhydramine is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the glofitamab or the obinutuzumab.

In some embodiments, the one or more additional therapeutic agents comprises granulocyte-colony stimulating factor (G-CSF). In some embodiments, G-CSF is administered between about one day and about two days after administration of any dose of rituximab, ifosfamide, carboplatin, and/or etoposide. In some embodiments, G-CSF is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day), or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In some embodiments, G-CSF is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

In some embodiments, the one or more additional therapeutic agents is an antipyretic. In some embodiments, the antipyretic is acetaminophen or paracetamol. In some embodiments, acetaminophen or paracetamol is administered orally or intravenously at a dose of between about 500 to about 1000 mg (e.g., 500, 550, 600, 650, 700, 750, 800, 850, 900, 950, or 1000 mg). In some embodiments, acetaminophen or paracetamol is administered at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the glofitamab or the obinutuzumab.

In some embodiments, the one or more additional therapeutic agents is mesna.

In some embodiments, mesna is administered intravenously at a dose of about 5000 mg/m² (e.g., 5000 mg/m²±50 mg/m², ±100 mg/m², ±200 mg/m², +300 mg/m², ±400 mg/m², or ±500 mg/m²). In some embodiments, mesna is administered via continuous infusion over about 24 hours on Day 3 of the first dosing cycle, on Day 6 of the second dosing cycle, and/or on Day 6 of each additional dosing cycle.

In some embodiments, the first dosing cycle comprises a first dose (C1D1) of ifosfamide, a second dose (C1D2) of ifosfamide, and a third dose (C1D3) of ifosfamide; a first dose (C1D1) of mesna, a second dose (C1D2) of mesna, and a third dose (C1D3) of mesna; a single dose (C1D1) of carboplatin; and a first dose (C1D1) of etoposide, a second dose (C1D2) of etoposide, and a third dose (C1D3) of etoposide; and wherein the second cycle comprises a first dose (C2D1) of ifosfamide, a second dose (C2D2) of ifosfamide, and a third dose (C2D3) of ifosfamide; a first dose (C2D1) of mesna, a second dose (C2D2) of mesna, and a third dose (C2D3) of mesna; a single dose (C2D1) of carboplatin; and a first dose (C2D1) of etoposide, a second dose (C2D2) of etoposide, and a third dose (C2D3) of etoposide.

In some embodiments, ifosfamide is administered at a dose of about 1666 mg/m² (e.g., 1666 mg/m²±25 mg/m², ±50 mg/m², +100 mg/m², or ±166.6 mg/m²) for each dose of ifosfamide, mesna is administered at a dose of about 1666 mg/m² (e.g., 1666 mg/m²±25 mg/m², ±50 mg/m², ±100 mg/m², or ±166.6 mg/m²) for each dose of mesna, carboplatin is administered at a dose in mg to target area under the curve (AUC) of about 5 mg/mL/min (e.g., 5 mg/mL/min±0.05 mg/mL/min, 0.1 mg/mL/min, 0.25 mg/mL/min, or +0.5 mg/mL/min) with maximum dose of about 800 mg (e.g., 800 mg±10 mg, ±25 mg, ±50 mg, or ±80 mg), and etoposide is administered at a dose of about 100 mg/m² for each dose of etoposide (e.g., 100 mg/m²±1 mg/m², ±2.5 mg/m², ±5 mg/m², or +10 mg/m²). In some embodiments, the maximum dose of carboplatin is about 750 mg (e.g., 750 mg±10 mg, ±25 mg, ±50 mg, or ±75 mg).

In some embodiments, carboplatin is administered on Day 2 of the first and second dosing cycles and the C1D1-C1D3 and C2D1-C2D3 of ifosfamide, mesna, and etoposide are administered on Days 1, 2, and 3, respectively, of each of the first and second dosing cycles.

In some embodiments, the first and second dosing cycles are each 21-day dosing cycles. In some embodiments, the dosing regimen comprises one or more additional dosing cycles. In some embodiments, the one or more additional dosing cycles are each 21-day dosing cycles.

In some embodiments, the dosing regimen comprises three dosing cycles in total.

In some embodiments, the one or more additional dosing cycles each comprises:
(a) an additional single dose of glofitamab;
(b) an additional single dose of rituximab; and
(c) an additional first dose, an additional second dose, and an additional third dose of ifosfamide; an additional first dose, an additional second dose, and an additional third dose of mesna; an additional single dose of carboplatin; and an additional first dose, an additional second dose, and an additional third dose of etoposide.

In some embodiments, the additional single dose of glofitamab is about 30 mg (e.g., 30 mg±0.1 mg, ±0.2 mg, ±0.3 mg, ±0.5 mg, ±0.75 mg, ±1 mg, ±1.5 mg, ±2 mg, or ±3 mg). In some embodiments, the additional single dose of glofitamab is administered to the individual on Day 8 of each of the one or more additional dosing cycles.

In some embodiments, the additional single dose of rituximab is about 375 mg/m². In some embodiments, the additional single dose of rituximab is administered on Day 1 of each of the one or more additional dosing cycles.

In some embodiments, ifosfamide is administered at a dose of about 1666 mg/m² (e.g., 1666 mg/m²±25 mg/m², ±50 mg/m², +100 mg/m², or ±166.6 mg/m²) for each dose of ifosfamide, mesna is administered at a dose of about 1666 mg/m² (e.g., 1666 mg/m²±25 mg/m², ±50 mg/m², ±100 mg/m², or ±166.6 mg/m²) for each dose of mesna, carboplatin is administered at a dose in mg to target area under the curve (AUC) of about 5 mg/mL/min (e.g., 5 mg/mL/min±0.05 mg/mL/min, 0.1 mg/mL/min, 0.25 mg/mL/min, or ±0.5 mg/mL/min) with maximum dose of about 750 mg (e.g., 750 mg±10 mg, ±25 mg, ±50 mg, or ±75 mg), and etoposide is administered at a dose of about 100 mg/m² for each dose of etoposide (e.g., 100 mg/m²±1 mg/m², ±2.5 mg/m², ±5 mg/m², or ±10 mg/m²).

In some embodiments, carboplatin is administered on Day 2 of each of the one or more additional dosing cycles and the additional first dose, the additional second dose, and the additional third dose of ifosfamide, mesna, and etoposide are administered on Days 1, 2, and 3, respectively, of each of the one or more additional dosing cycles.

In some embodiments, rituximab, ifosfamide, mesna, carboplatin, and etoposide are administered in an outpatient setting. In some embodiments, each dose of mesna is administered simultaneously with the corresponding dose of ifosfamide on any day of a dosing cycle comprising a dose of mesna and a dose of ifosfamide.

In some instances, glofitamab, rituximab, ifosfamide, mesna, carboplatin, and etoposide are administered to the induvial as salvage treatment prior to autologous stem cell transplant (ASCT) or CAR-T cell therapy.

In one embodiment, the invention features glofitamab for use in treating relapsed or refractory diffuse large B-cell lymphoma (DLBCL) in a patient eligible for autologous stem cell transplant or CAR-T cell therapy, comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle,
wherein the treatment results in an objective response rate of at least 80% in a plurality of treated patients.

In one embodiment, said objective response rate is reached within 3 cycles of Glofit-R-ICE and prior to ASCT or CAR T-cell therapy, based on Lugano criteria.

In one embodiment, the invention features glofitamab for use in treating relapsed diffuse large B-cell lymphoma (DLBCL) in a patient eligible for autologous stem cell transplant or CAR-T cell therapy, wherein the patient has relapsed less than 12 months after receiving prior therapy, comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle.
   In one embodiment, the invention features glofitamab for use in treating relapsed diffuse large B-cell lymphoma (DLBCL) in a patient eligible for autologous stem cell transplant or CAR-T cell therapy, wherein the patient has relapsed more than 12 months after receiving prior therapy, comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle.

In one embodiment, the invention features glofitamab for use in treating refractory diffuse large B-cell lymphoma (DLBCL) in a patient eligible for autologous stem cell transplant or CAR-T cell therapy, comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle.

In one embodiment, the invention features glofitamab for use in treating relapsed or refractory diffuse large B-cell lymphoma (DLBCL) in a patient eligible for autologous stem cell transplant or CAR-T cell therapy, comprising 3 treatment cycles, wherein
a) Treatment cycle 1 comprises administering
   Obinutuzumab at a dose of 1000 mg on day 1 of cycle 1,
   Ifosfamide either at a dose of 5000 mg/m² on day 2 cycle 1 or at a dose of 1666 mg/m² on days 1, 2 and 3 of cycle 1
   Carboplatin at a dose of 5 mg/mL/min×(25+ creatine clearance, calculated using Cockcroft-Gault equation) and capped at 750 mg on day 2 of cycle 1,
   Etoposide or etoposide phosphate at a dose of 75-100 mg/m² on days 1, 2 and 3, and
   Glofitamab at a dose of 2.5 mg on day 8 and at a dose of 10 mg on day 15 of cycle 1
b) Treatment cycles 2 and 3 comprise administering
   Ifosfamide either at a dose of 5000 mg/m² on day 2 cycle 1 or at a dose of 1666 mg/m² on days 1, 2 and 3 of the respective cycle,
   Carboplatin at a dose of 5 mg/mL/min×(25+ creatine clearance, calculated using Cockcroft-Gault equation) and capped at 750 mg on day 2 of the respective cycle,
   Etoposide or etoposide phosphate at a dose of 75-100 mg/m² on days 1, 2 and 3 of the respective cycle, and administering 30 mg glofitamab on day 8 of the respective cycle.
wherein the treatment results in an objective response rate of at least 80% in a plurality of treated patients.

In one embodiment, said objective response rate is reached within 3 cycles of Glofit-R-ICE and prior to ASCT or CAR T-cell therapy, based on Lugano criteria.

In one embodiment, the invention features glofitamab for use in treating relapsed diffuse large B-cell lymphoma (DLBCL) in a patient eligible for autologous stem cell transplant or CAR-T cell therapy, wherein the patient has relapsed less than 12 months after receiving prior therapy, comprising 3 treatment cycles, wherein
a) Treatment cycle 1 comprises administering
   Obinutuzumab at a dose of 1000 mg on day 1 of cycle 1,
   Ifosfamide either at a dose of 5000 mg/m² on day 2 cycle 1 or at a dose of 1666 mg/m² on days 1, 2 and 3 of cycle 1
   Carboplatin at a dose of 5 mg/mL/min×(25+ creatine clearance, calculated using Cockcroft-Gault equation) and capped at 750 mg on day 2 of cycle 1,
   Etoposide or etoposide phosphate at a dose of 75-100 mg/m² on days 1, 2 and 3, and
   Glofitamab at a dose of 2.5 mg on day 8 and at a dose of 10 mg on day 15 of cycle 1
b) Treatment cycles 2 and 3 comprise administering
   Ifosfamide either at a dose of 5000 mg/m² on day 2 cycle 1 or at a dose of 1666 mg/m² on days 1, 2 and 3 of the respective cycle,
   Carboplatin at a dose of 5 mg/mL/min×(25+ creatine clearance, calculated using Cockcroft-Gault equation) and capped at 750 mg on day 2 of the respective cycle,
   Etoposide or etoposide phosphate at a dose of 75-100 mg/m² on days 1, 2 and 3 of the respective cycle, and administering 30 mg glofitamab on day 8 of the respective cycle.

In one embodiment, the invention features glofitamab for use in treating relapsed large B-cell lymphoma (DLBCL) in a patient eligible for autologous stem cell transplant or CAR-T cell therapy, wherein the patient has relapsed more than 12 months after receiving prior therapy, comprising 3 treatment cycles, wherein
a) Treatment cycle 1 comprises administering
   Obinutuzumab at a dose of 1000 mg on day 1 of cycle 1,
   Ifosfamide either at a dose of 5000 mg/m² on day 2 cycle 1 or at a dose of 1666 mg/m² on days 1, 2 and 3 of cycle 1
   Carboplatin at a dose of 5 mg/mL/min×(25+ creatine clearance, calculated using Cockcroft-Gault equation) and capped at 750 mg on day 2 of cycle 1,
   Etoposide or etoposide phosphate at a dose of 75-100 mg/m² on days 1, 2 and 3, and
   Glofitamab at a dose of 2.5 mg on day 8 and at a dose of 10 mg on day 15 of cycle 1
b) Treatment cycles 2 and 3 comprise administering
   Ifosfamide either at a dose of 5000 mg/m² on day 2 cycle 1 or at a dose of 1666 mg/m² on days 1, 2 and 3 of the respective cycle,
   Carboplatin at a dose of 5 mg/mL/min×(25+ creatine clearance, calculated using Cockcroft-Gault equation) and capped at 750 mg on day 2 of the respective cycle,
   Etoposide or etoposide phosphate at a dose of 75-100 mg/m² on days 1, 2 and 3 of the respective cycle, and administering 30 mg glofitamab on day 8 of the respective cycle.

In one embodiment, the invention features glofitamab for use in treating refractory diffuse large B-cell lymphoma (DLBCL) in a patient eligible for autologous stem cell transplant or CAR-T cell therapy, comprising 3 treatment cycles, wherein
a) Treatment cycle 1 comprises administering
   Obinutuzumab at a dose of 1000 mg on day 1 of cycle 1,
   Ifosfamide either at a dose of 5000 mg/m² on day 2 cycle 1 or at a dose of 1666 mg/m² on days 1, 2 and 3 of cycle 1
   Carboplatin at a dose of 5 mg/mL/min×(25+ creatine clearance, calculated using Cockcroft-Gault equation) and capped at 750 mg on day 2 of cycle 1,
   Etoposide or etoposide phosphate at a dose of 75-100 mg/m² on days 1, 2 and 3, and
   Glofitamab at a dose of 2.5 mg on day 8 and at a dose of 10 mg on day 15 of cycle 1
b) Treatment cycles 2 and 3 comprise administering
   Ifosfamide either at a dose of 5000 mg/m² on day 2 cycle 1 or at a dose of 1666 mg/m² on days 1, 2 and 3 of the respective cycle,
   Carboplatin at a dose of 5 mg/mL/min×(25+ creatine clearance, calculated using Cockcroft-Gault equation) and capped at 750 mg on day 2 of the respective cycle,
   Etoposide or etoposide phosphate at a dose of 75-100 mg/m² on days 1, 2 and 3 of the respective cycle, and administering 30 mg glofitamab on day 8 of the respective cycle.

In one embodiment, the invention features glofitamab for use in treating relapsed diffuse large B-cell lymphoma (DLBCL) in a patient eligible for autologous stem cell transplant or CAR-T cell therapy comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle, wherein the treatment results in an objective response rate of at least 85% in a plurality of treated patients.

In one embodiment, the invention features glofitamab for use in treating relapsed diffuse large B-cell lymphoma (DLBCL) in a patient eligible for autologous stem cell transplant or CAR-T cell therapy comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle, wherein the treatment results in a complete response rate of at least 70% in a plurality of treated patients.

In one embodiment, the invention features glofitamab for use in treating refractory diffuse large B-cell lymphoma (DLBCL) in a patient eligible for autologous stem cell transplant or CAR-T cell therapy comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle, wherein the treatment results in an objective response rate of at least 75% in a plurality of treated patients.

In one embodiment, the invention features glofitamab for use in treating refractory diffuse large B-cell lymphoma (DLBCL) in a patient eligible for autologous stem cell transplant or CAR-T cell therapy comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle, wherein the treatment results in a complete response rate of at least 50% in a plurality of treated patients.

In one embodiment, said objective response rate or complete response rate is reached within 3 cycles of Glofit-R-ICE and prior to ASCT or CAR T-cell therapy, based on Lugano criteria.

In one embodiment, the invention features glofitamab for use in treating relapsed diffuse large B-cell lymphoma (DLBCL) in a patient eligible for autologous stem cell transplant or CAR-T cell therapy of any of the embodiments above, comprising 3 treatment cycles, wherein
a) Treatment cycle 1 comprises administering
   Obinutuzumab at a dose of 1000 mg on day 1 of cycle 1,
   Ifosfamide either at a dose of 5000 mg/m² on day 2 cycle 1 or at a dose of 1666 mg/m² on days 1, 2 and 3 of cycle 1
   Carboplatin at a dose of 5 mg/mL/min×(25+ creatine clearance, calculated using Cockcroft-Gault equation) and capped at 750 mg on day 2 of cycle 1,
   Etoposide or etoposide phosphate at a dose of 75-100 mg/m² on days 1, 2 and 3, and
   Glofitamab at a dose of 2.5 mg on day 8 and at a dose of 10 mg on day 15 of cycle 1
b) Treatment cycles 2 and 3 comprise administering
   Ifosfamide either at a dose of 5000 mg/m² on day 2 cycle 1 or at a dose of 1666 mg/m² on days 1, 2 and 3 of the respective cycle,
   Carboplatin at a dose of 5 mg/mL/min×(25+ creatine clearance, calculated using Cockcroft-Gault equation) and capped at 750 mg on day 2 of the respective cycle,
   Etoposide or etoposide phosphate at a dose of 75-100 mg/m² on days 1, 2 and 3 of the respective cycle, and administering 30 mg glofitamab on day 8 of the respective cycle.
wherein the treatment results in an objective response rate of at least 80% in a plurality of treated patients.

In one embodiment, the invention features glofitamab for use in treating relapsed or refractory diffuse large B-cell lymphoma (DLBCL) in a patient eligible for autologous stem cell transplant or CAR-T cell therapy according to any of the embodiments above, wherein the treatment does not result in any grade 3 or higher CRS.

In one embodiment, the treatment does not result in any grade 2 or higher CRS as of cycle 2.

In one embodiment, glofitamab is provided for use in treating relapsed or refractory diffuse large B-cell lymphoma (DLBCL) in a patient eligible for autologous stem cell transplant or CAR-T cell therapy according to any of the embodiments above, wherein the treatment comprises two additional cycles with 30 mg glofitamab monotherapy. In one embodiment, glofitamab is administered on day 8 of additional cycles 4 and 5.

In one embodiment, glofitamab is provided for use in treating relapsed or refractory diffuse large B-cell lymphoma (DLBCL) in a patient eligible for autologous stem cell transplant or CAR-T cell therapy according to any of the embodiments above, wherein the DLBCL is high-grade B-cell lymphoma (HGBCL) NOS or HGBCL with MYC and BCL-2/6.

In one embodiment, glofitamab is provided for use in treating relapsed or refractory diffuse large B-cell lymphoma (DLBCL) in a patient eligible for autologous stem cell transplant or CAR-T cell therapy according to any of the embodiments above, wherein the patient has received one prior therapy. In one embodiment, glofitamab is provided for use in treating relapsed or refractory diffuse large B-cell lymphoma (DLBCL) in a patient eligible for autologous stem cell transplant or CAR-T cell therapy according to any of the embodiments above, wherein the patient has Eastern Cooperative Oncology Group performance status 0 or 1.

In one embodiment, glofitamab is provided for use in treating relapsed or refractory diffuse large B-cell lymphoma (DLBCL) in a patient eligible for autologous stem cell transplant or CAR-T cell therapy according to any of the embodiments above, wherein the cell of origin is activated B-cell-like or germinal center B-cell-like.

### Therapeutic Agents for Use in the Methods of the Invention

### Glofitamab

In one embodiment, the combination treatment described herein includes the anti-CD20/anti-CD3 bispecific antibody glofitamab. Glofitamab (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, RO7082859, or RG6026; CAS #: 2229047-91-8) is a novel T-cell-engaging bispecific (TCB) full-length antibody with a 2:1 molecular configuration for bivalent binding to CD20 on B cells and monovalent binding to CD3, particularly the CD3 epsilon chain (CD3e), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3e of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. The sequences of glofitamab are summarized in Table 2.

TABLE 2
Sequence IDs for glofitamab
Sequence IDs for glofitamab

| SEQ ID NO: | Description | SEQ ID NO: | Description |
|---|---|---|---|
| CD3 Heavy Chain | | CD3 Light Chain | |
| 9 | HVR-H1 (Kabat) | 12 | HVR-L1 (Kabat) |
| 10 | HVR-H2 (Kabat) | 13 | HVR-L2 (Kabat) |
| 11 | HVR-H3 (Kabat) | 14 | HVR-L3 (Kabat) |
| 15 | VH | 16 | VL |
| CD20 Heavy Chain | | CD20 Light Chain | |
| 1 | HVR-H1 (Kabat) | 4 | HVR-L1 (Kabat) |
| 2 | HVR-H2 (Kabat) | 5 | HVR-L2 (Kabat) |
| 3 | HVR-H3 (Kabat) | 6 | HVR-L3 (Kabat) |
| 7 | VH | 8 | VL |

Full-length antibody

| | | | |
|---|---|---|---|
| 17 | HC-knob | 18 | HC-hole |
| 19 | LC-CD3 | 20 | LC-CD20 |

### Rituximab, Ifosfamide, Carboplatin, and Etoposide

In some instances, the combination treatment described herein includes the type I anti-CD20 antibody rituximab (RITUXAN®), which has the CAS #: 174722-31-7. In some instances, the combination treatment described herein includes ifosfamide, which has the CAS #: 3778-73-2. In some instances, the combination treatment described herein includes carboplatin, which has the CAS #: 41575-94-4. In some instances, the combination treatment described herein includes etoposide, which has the CAS #: 33419-42-0.

### Obinutuzumab

In some instances, the combination treatment described herein includes the type II anti-CD20 antibody obinutuzumab (GAZYVA®), which has the CAS #: 949142-50-1.

### (iv) CRS Risk Mitigation Strategies

The present invention relates to a combination treatment including glofitamab in combination with rituximab, ifosfamide, carboplatin, and etoposide (R-ICE).

Bispecific antibody therapeutics involving T-cell activation (e.g., glofitamab) have been associated with cytokine release syndrome (CRS). CRS is a potentially life-threatening symptom complex caused by the excessive release of cytokines by immune effector or target cells during an exaggerated and sustained immune response. CRS can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or fatal outcome. Current clinical management focuses on treating the individual signs and symptoms, providing supportive care, and attempting to dampen down the inflammatory response using high-dose corticosteroids. However, this approach is not always successful, especially in the case of late intervention. Moreover, steroids may negatively impact T-cell function, which may diminish the clinical benefit of immune modulating therapies in the treatment of cancer.

### A. CRS Symptoms and Grading

CRS is graded according to the Modified Cytokine Release Syndrome Grading System established by Lee et al., Blood, 124: 188-195, 2014 or Lee et al., Biol Blood Marrow Transplant, 25(4): 625-638, 2019, as described in Table 4. In addition to diagnostic criteria, recommendations on management of CRS based on its severity, including early intervention with corticosteroids and/or anti-cytokine therapy, are provided and referenced in Tables 4 and 5.

TABLE 4
Cytokine release syndrome grading systems

| | Modified Cytokine Release | ASTCT Consensus Grading |
|---|---|---|
| Grade | Syndrome Grading System | System |
| Grade 1 | Symptoms are not life | Temperature ≥38° C. |
| | threatening and require | No hypotension |
| | symptomatic treatment only | No hypoxia |
| | (e.g., fever, nausea, fatigue, | |
| | headache, myalgia, malaise) | |
| Grade 2 | Symptoms require and respond | Temperature ≥38° C.* with |
| | to moderate intervention | hypotension not requiring |
| | Oxygen requirement <40%; or | vasopressors and/or^{†} hypoxia |
| | Hypotension responsive to fluids | requiring low-flow nasal |
| | or low dose^{a} of one vasopressor; | cannula^{‡} or blow-by |
| | or Grade 2 organ toxicity | |
| Grade 3 | Symptoms require and respond | Temperature ≥38° C.* with |
| | to aggressive intervention | hypotension requiring a |
| | Oxygen requirement ≥40%; or | vasopressor with or without |
| | Hypotension requiring high | vasopressin and/or^{†} hypoxia |
| | dose^{b} or multiple | requiring high-flow nasal |
| | vasopressors; or Grade 3 organ | cannula^{‡}, facemask, |
| | toxicity or Grade 4 transaminitis | nonrebreather mask, or |
| | | Venturi mask |
| Grade 4 | Life-threatening symptoms | Temperature ≥38° C.* with |
| | Requirement for ventilation | hypotension requiring |
| | support or | multiple vasopressors |
| | Grade 4 organ toxicity | (excluding vasopressin) |
| | (excluding transaminitis) | and/or^{†} hypoxia |
| | | requiring positive pressure |
| | | (e.g., CPAP, BiPAP, |
| | | intubation and mechanical |
| | | ventilation) |
| Grade 5 | Death | Death |

Lee 2014 criteria: Lee et al., Blood, 124: 188-195, 2014.
ASTCT consensus grading: Lee et al., Biol Blood Marrow Transplant, 25(4): 625-638, 2019.
^{a}Low-dose vasopressor: single vasopressor at doses below that shown in Table 4.
^{b}High-dose vasopressor: as defined in Table 4.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 5
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent of ≥10 μg/min ^{a} |
| If on combination or vasopressors | Norepinephrine equivalent |
| (not vasopressin) | of ≥20 μg/min ^{a} |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124: 188-195, 2014; Maude et al., New Engl J Med, 371: 1507-1517, 2014).

B. Pretreatment or Management of CRS Related Symptoms with Tocilizumab

CRS is associated with high IL-6 levels (Panelli et al., J Transl Med, 2: 17, 2004; Lee et al., Blood, 124: 188-195, 2014; Doessegger and Banholzer, Clin Transl Immunology, 4: e39, 2015), and IL-6 correlates with the severity of CRS, with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J Immunol Methods, 434: 1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling (see, e.g., WO 1992/019579, which is incorporated herein by reference in its entirety). Tocilizumab has been approved by the U.S. Food and Drug Administration for the treatment of severe or life-threatening CAR-T cell-induced CRS in adults and in pediatric patients 2 years of age and older. Initial clinical data (Locke et al., Blood, 130: 1547, 2017) suggests that tocilizumab prophylaxis may reduce the severity of CAR-T cell-induced CRS by blocking IL-6 receptors from signaling prior to cytokine release. Consequently, tocilizumab premedication may also reduce the frequency or lower the severity of CRS associated with bispecific antibody (e.g., glofitamab) therapy. Other anti-IL-6R antibodies that could be used in combination with tocilizumab include sarilumab, vobarilizumab (ALX-0061), SA-237, and variants thereof.

In some aspects, an effective amount of tocilizumab is administered as a premedication, e.g., is administered to the individual prior to the administration of glofitamab. Administration of tocilizumab as a premedication may reduce the frequency or severity of CRS. In some aspects, tocilizumab is administered as a premedication in Cycle 1, e.g., is administered prior to a first dose (C1D1), a second dose (C1D2), and/or a third dose (C1D3) of glofitamab. In some aspects, tocilizumab is administered intravenously to the individual as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, tocilizumab is administered intravenously to the individual as a single dose of about 8 mg/kg. Other anti-IL-6R antibodies that could be used in combination with tocilizumab include sarilumab, vobarilizumab (ALX-0061), SA-237, and variants thereof.

For example, in one aspect, glofitamab is co-administered with tocilizumab (ACTEMRA®/ROACTEMRA®), wherein the individual is first administered with tocilizumab (ACTEMRA®/ROACTEMRA®) and then separately administered with the bispecific antibody (e.g., the individual is pre-treated with tocilizumab (ACTEMRA®/ROACTEMRA®)).

In another aspect, tocilizumab is administered to treat or alleviate symptoms associated with CRS in individuals treated with glofitamab. If the individual has a grade 2 or higher CRS event in the presence of extensive comorbidities following administration of glofitamab, the method may further include administering to the individual a first dose of an IL-6R antagonist (e.g., an anti-IL-6R antibody, e.g., tocilizumab (ACTEMRA®/ROACTEMRA®)) to manage the grade 2 or higher CRS event while suspending treatment with glofitamab. In some instances, the first dose of tocilizumab is administered intravenously to the individual at a dose of about 8 mg/kg. Other anti-IL-6R antibodies that could be used in combination with tocilizumab include sarilumab, vobarilizumab (ALX-0061), SA-237, and variants thereof. In some instances, if the grade 2 or higher CRS event resolves to a grade ≤1 CRS event within two weeks, the method further includes resuming treatment with glofitamab at a reduced dose. In some instances, the reduced dose is 50% of the initial infusion rate of the previous cycle if the event occurred during or within 24 hours of the infusion. If, on the other hand, the grade 2 or higher CRS event does not resolve or worsens to a grade ≥3 CRS event within 24 hours of treating the symptoms of the grade 2 or higher CRS event, the method may further include administering to the individual one or more (e.g., one, two, three, four, or five or more) additional doses of an IL-6R antagonist (e.g., an anti-IL-6R antibody, e.g., tocilizumab) to manage the grade 2 or grade ≥3 CRS event. In some particular instances, the grade 2 or higher CRS event does not resolve or worsens to a grade ≥3 CRS event within 24 hours of treating the symptoms of the grade 2 or higher CRS event, and the method may further include administering to the individual one or more additional doses of tocilizumab to manage the grade 2 or grade ≥3 CRS event. In some instances, the one or more additional doses of tocilizumab is administered intravenously to the individual at a dose of about 1 mg/kg to about 15 mg/kg, e.g., about 4 mg/kg to about 10 mg/kg, e.g., about 6 mg/kg to about 10 mg/kg, e.g., about 8 mg/kg.

### C. Other Pretreatments for CRS Risk Mitigation

In one embodiment, the treatment regimen provided herein further comprises administration of premedication prior to the administration of glofitamab. In one embodiment the premedication comprises a corticosteroid (such as, e.g., prednisolone, dexamethasone, or methylprednisolone), paracetamol/acetaminophen, and/or an anti-histamine (such as, e.g., diphenhydramine). In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the treatment regimen further comprises administration of premedication prior to the administration of glofitamab. In embodiment the premedication comprises a corticosteroid (such as, e.g., prednisolone, dexamethasone, or methylprednisolone), an anti-pyretic (such as, e.g., paracetamol/acetaminophen), and/or an anti-histamine (such as, e.g., diphenhydramine). In one embodiment, the individual receives corticosteroid premedication prior to administration of glofitamab. It has been shown that premedication using dexamethasone reduced glofitamab-induced cytokine levels in mice pretreated with dexamethasone relative to methylprednisolone. Therefore, in one embodiment, the corticosteroid is dexamethasone. In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the premedication is administered at least 60 minutes (e.g., at least 2, 3, 4, 5, 6, 7, 8, 9, 10, 11, 12, 18, 24, 36, 48 hours, or more) prior to each administration of glofitamab. In another embodiment, pre-medication with corticosteroids is administered before the first dose (C1D1) and second dose (C1D2) of the first cycle, before the first dose of the second (C2D1) and third (C3D1) cycle and may be optional for subsequent cycles where the target dose has been reached and tolerated for two doses for patients with no CRS in previous cycles.

In one embodiment, the premedication is administered at least 60 minutes (e.g., at least 2, 3, 4, 5, 6, 7, 8, 9, 10, 11, 12, 18, 24, 36, 48 hours, or more) prior to the administration of the pretreatment with the anti-CD20 antibody, particularly obinutuzumab.

In one embodiment, corticosteroids are administered to manage any relevant adverse events arising after administration of glofitamab.

### (v) Administration of Therapeutic Agents

The methods may involve administering glofitamab (and/or any additional therapeutic agent) by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, glofitamab is administered by intravenous infusion.

In one embodiment the infusion time for glofitamab, particularly glofitamab, is at least 4 hours (e.g., about 4 hours, about 4.5 hours, about 5 hours, about 5.5 hours, or about 6 hours). In a particular embodiment, the infusion duration for glofitamab is about 4 hours. In one embodiment the infusion time for glofitamab may be reduced or extended. In one embodiment (for example, in the absence of infusion-related adverse events), the infusion time of glofitamab in subsequent cycles is reduced to 2 hours±15 minutes. In one embodiment the infusion time is increased to up to 8 hours (e.g., about 4 hours, about 5 hours, about 6 hours, about 7 hours, or about 8 hours) (for example, for individuals with high risk of experiencing CRS). In one embodiment, for example, for patients who may be at an increased risk of CRS, patients who experience IRRs or CRS with their previous dose of glofitamab or who are at increased risk of recurrent IRR/CRS with subsequent doses, the time of infusion of glofitamab is extended to up to 8 hours.

For all the methods described herein, glofitamab, obinutuzumab, rituximab, ifosfamide, carboplatin, or etoposide, and/or one or more additional therapeutic agents described herein would be formulated, dosed, and administered in a fashion consistent with good medical practice. Factors for consideration in this context include the particular disorder being treated, the particular mammal being treated, the clinical condition of the individual or subject, the cause of the disorder, the site of delivery of the agent, the method of administration, the scheduling of administration, and other factors known to medical practitioners. glofitamab needs not be, but is optionally formulated with, one or more agents currently used to prevent or treat the disorder in question. The effective amount of such other agents depends on the amount of glofitamab present in the formulation, the type of disorder or treatment, and other factors discussed above. glofitamab may be suitably administered to the individual over a series of treatments.

A further aspect of the present invention relates to the invention as described hereinbefore.

### Embodiments

Some embodiments of the technology described herein can be defined according to any of the following numbered embodiments:
1. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in complete response (CR) rate of a plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.
2. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in complete response (CR) rate of a plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.
3. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in complete response (CR) rate of a plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab.
4. The method, use, or glofitamab for use of any one of embodiments 1 to 33, wherein the improvement in CR rate is between 8% and 44%.
5. The method, use, or glofitamab for use of embodiment 4, wherein the improvement in CR rate is an increase of about 28%.
6. The method, use, or glofitamab for use of any one of embodiments 1 to 5, wherein the improvement in CR rate is statistically significant.
7. A method for treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in a CR rate of between 45% and 83%.
8. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of individuals results in a CR rate of between 45% and 83%.
9. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of individuals results in a CR rate of between 45% and 83%.
10. The method, use, or glofitamab for use of any one of embodiments 7 to 9, wherein the CR rate is about 66%, 67%, or 69%.
11. The method, use, or glofitamab for use of any one of embodiments 1 to 10, wherein the CR rate is the proportion of individuals whose best overall response is a CR as determined by positron emission tomography-computed tomography (PET-CT) or positron emission tomography-magnetic resonance imaging (PET-MRI).
12. The method, use, or glofitamab for use of embodiment 11, wherein the CR is determined according to the Lugano Criteria, Cheson et al. J Clin Oncol. 2014 Sep. 20; 32(27): 3059-3067.
13. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in overall response (OR) rate of a plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.
14. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in overall response (OR) rate of a plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.
15. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in overall response (OR) rate of a plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab.
16. The method, use, or glofitamab for use of any one of embodiments 13 to 15, wherein the improvement in OR rate is between 1% and 29%.
17. The method, use, or glofitamab for use of embodiment 16, wherein the improvement in OR rate is an increase of about 15%.
18. The method, use, or glofitamab for use of any one of embodiments 13 to 17, wherein the improvement in OR rate is statistically significant.
19. A method for treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of individuals results in an OR rate of between 56% and 90%.
20. Glofitamab for use in a method of treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in an OR rate of between 56% and 90%.
21. Use of glofitamab in the manufacture of a medicament for treating relapsed or refractory DLBCL in a human individual in need thereof, comprising administering to the human individual an effective amount of glofitamab, rituximab, ifosfamide, carboplatin, and etoposide, wherein administering such treatment to a plurality of individuals results in an OR rate of between 56% and 90%.
22. The method, use, or glofitamab for use of any one of embodiments 19 to 21, wherein the OR rate is about 76%, 78%, or 83%.
23. The method, use, or glofitamab for use of any one of embodiments 13 to 22, wherein the OR rate is the proportion of individuals whose best overall response is a partial response (PR) or a CR.
24. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in median progression free survival (PFS) of a plurality of human individuals as compared to a reference median PFS, wherein the reference median PFS is the median PFS of a plurality of human individuals who have received a control treatment comprising rituximab, ifosfamide, carboplatin, and etoposide in the absence of glofitamab.
25. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median PFS of a plurality of human individuals as compared to a reference median PFS, wherein the reference median PFS is the median PFS of a plurality of human individuals who have received a control treatment comprising rituximab, ifosfamide, carboplatin, and etoposide in the absence of glofitamab.
26. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median PFS of a plurality of human individuals as compared to a reference median PFS, wherein the reference median PFS is the median PFS of a plurality of human individuals who have received a control treatment comprising rituximab, ifosfamide, carboplatin, and etoposide in the absence of glofitamab.
27. The method, use, or glofitamab for use of any one of embodiments 24 to 26, wherein PFS is defined as the time from first study treatment to the first occurrence of disease progression or death from any cause.
28. The method, use, or glofitamab for use of any one of embodiments 24 to 26, wherein PFS is determined according to the Lugano criteria.
29. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in median event free survival (EFS) of a plurality of human individuals as compared to a reference median EFS, wherein the reference median EFS is the median EFS of a plurality of human individuals who have received a control treatment comprising rituximab, ifosfamide, carboplatin, and etoposide in the absence of glofitamab.
30. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median EFS of a plurality of human individuals as compared to a reference median EFS, wherein the reference median EFS is the median EFS of a plurality of human individuals who have received a control treatment comprising rituximab, ifosfamide, carboplatin, and etoposide in the absence of glofitamab.
31. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median EFS of a plurality of human individuals as compared to a reference median EFS, wherein the reference median EFS is the median EFS of a plurality of human individuals who have received a control treatment comprising rituximab, ifosfamide, carboplatin, and etoposide in the absence of glofitamab.
32. The method, use, or glofitamab for use of any one of embodiments 29 to 31, wherein EFS is defined as the time from first study treatment to the first occurrence of disease progression, initiation of new anti-lymphoma therapy, or death from any cause.
33. The method, use, or glofitamab for use of any one of embodiments 29 to 32, wherein EFS is determined according to the Lugano criteria.
34. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in mobilization-adjusted response rate (MARR) of a plurality of human individuals as compared to a reference MARR, wherein the reference MARR is the MARR of a plurality of human individuals who have received a control treatment comprising rituximab, ifosfamide, carboplatin, and etoposide in the absence of glofitamab.
35. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in MARR of a plurality of human individuals as compared to a reference MARR, wherein the reference MARR is the MARR of a plurality of human individuals who have received a control treatment comprising rituximab, ifosfamide, carboplatin, and etoposide in the absence of glofitamab.
36. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in MARR of a plurality of human individuals as compared to a reference MARR, wherein the reference MARR is the MARR of a plurality of human individuals who have received a control treatment comprising rituximab, ifosfamide, carboplatin, and etoposide in the absence of glofitamab.
37. The method, use, or glofitamab for use of any one of embodiments 34 to 36, wherein MARR is defined as the rate of individuals treated with intent to proceed to autologous stem cell transplant (ASCT) therapy that achieve a CR or PR within three cycles of treatment, and additionally achieves mobilization of a minimum of 2,000,000 CD34+ hematopoietic stem cells/kg for ASCT.
38. The method, use, or glofitamab for use of any one of embodiments 34 to 37, wherein CR or PR is determined according to the Lugano criteria.
39. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in median duration of response (DOR) of a plurality of human individuals as compared to a reference median DOR, wherein the reference median DOR is the median DOR of a plurality of human individuals who have received a control treatment comprising rituximab, ifosfamide, carboplatin, and etoposide in the absence of glofitamab.
40. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median DOR of a plurality of human individuals as compared to a reference median DOR, wherein the reference median DOR is the median DOR of a plurality of human individuals who have received a control treatment comprising rituximab, ifosfamide, carboplatin, and etoposide in the absence of glofitamab.
41. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median DOR of a plurality of human individuals as compared to a reference median DOR, wherein the reference median DOR is the median DOR of a plurality of human individuals who have received a control treatment comprising rituximab, ifosfamide, carboplatin, and etoposide in the absence of glofitamab.
42. The method, use, or glofitamab for use of any one of embodiments 39 to 41, wherein DOR is defined as the time from first documented CR or PR to disease progression or death from any cause.
43. The method, use, or glofitamab for use of any one of embodiments 39 to 42, wherein DOR is determined according to the Lugano criteria.
44. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in median duration of complete response (DOCR) of a plurality of human individuals as compared to a reference median DOCR, wherein the reference median DOCR is the median DOCR of a plurality of human individuals who have received a control treatment comprising rituximab, ifosfamide, carboplatin, and etoposide in the absence of glofitamab.
45. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median DOCR of a plurality of human individuals as compared to a reference median DOCR, wherein the reference median DOCR is the median DOCR of a plurality of human individuals who have received a control treatment comprising rituximab, ifosfamide, carboplatin, and etoposide in the absence of glofitamab.
46. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median DOCR of a plurality of human individuals as compared to a reference median DOCR, wherein the reference median DOCR is the median DOCR of a plurality of human individuals who have received a control treatment comprising rituximab, ifosfamide, carboplatin, and etoposide in the absence of glofitamab.
47. The method, use, or glofitamab for use of any one of embodiments 44 to 46, wherein DOCR is defined as the time from first documented CR to disease progression or death from any cause.
48. The method, use, or glofitamab for use of any one of embodiments 44 to 47, wherein DOCR is determined according to the Lugano criteria.
49. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in an improvement in median overall survival (OS) of a plurality of human individuals as compared to a reference median OS, wherein the reference median OS is the median OS of a plurality of human individuals who have received a control treatment comprising rituximab, ifosfamide, carboplatin, and etoposide in the absence of glofitamab.
50. Glofitamab for use in a method of treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median OS of a plurality of human individuals as compared to a reference median OS, wherein the reference median OS is the median OS of a plurality of human individuals who have received a control treatment comprising rituximab, ifosfamide, carboplatin, and etoposide in the absence of glofitamab.
51. Use of glofitamab in the manufacture of a medicament for treating relapsed or refractory diffuse large B-cell lymphoma (DLBCL) in a human individual in need thereof, wherein a treatment comprising an effective amount of glofitamab, rituximab, ifosfamide, carboplatin, and etoposide is to be administered to the human individual,
wherein administering such treatment to a plurality of human individuals results in an improvement in median OS of a plurality of human individuals as compared to a reference median OS, wherein the reference median OS is the median OS of a plurality of human individuals who have received a control treatment comprising rituximab, ifosfamide, carboplatin, and etoposide in the absence of glofitamab.
52. The method, use, or glofitamab for use of any one of embodiments 49 to 51, wherein OS is defined as the time from first study treatment to death from any cause.
53. The method, use, or glofitamab for use of any one of embodiments 1 to 52, wherein the treatment comprises administering glofitamab, rituximab, ifosfamide, carboplatin, and etoposide according to a dosing regimen comprising a first and a second dosing cycle, wherein:
the first dosing cycle comprises a first dose (C1D1) of about 2.5 mg of the glofitamab and a second dose (C1D2) of about 10 mg of the glofitamab, and
the second dosing cycle comprises a single dose (C2D1) of about 30 mg of the glofitamab.
54. The method, use, or glofitamab for use of embodiment 53, wherein the C1D1 and the C1D2 of the glofitamab are administered to the individual on Days 8 and 15, respectively, of the first dosing cycle.
55. The method, use, or glofitamab for use of embodiment 53 or 54, wherein the C2D1 of the glofitamab is administered to the individual on Day 8 of the second dosing cycle.
56. The method, use, or glofitamab for use of any one of embodiments 53 to 55, wherein the first dosing cycle comprises a single dose (C1D1) of obinutuzumab.
57. The method, use, or glofitamab for use of embodiment 56, wherein the C1D1 of obinutuzumab is about 1000 mg.
58. The method, use, or glofitamab for use of embodiment 56 or 57, wherein the C1D1 of obinutuzumab is administered on Day 1 of the first dosing cycle.
59. The method, use, or glofitamab for use of any one of embodiments 53 to 58, wherein the second dosing cycle comprises a single dose (C2D1) of rituximab.
60. The method, use, or glofitamab for use of embodiment 59, wherein the C2D1 of rituximab is about 375 mg/m².
61. The method, use, or glofitamab for use of embodiment 59 or 60, wherein the C2D1 of rituximab is administered on Day 1 of the second dosing cycle.
62. The method, use, or glofitamab for use of any one of embodiments 1 to 61, wherein the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and
wherein the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.
63. The method, use, or glofitamab for use of embodiment 62, wherein ifosfamide is administered at a dose of about 5000 mg/m², carboplatin is administered at a dose in mg to target area under the curve (AUC) of about 5 mg/mL/min with maximum dose of about 750 mg, and etoposide is administered at a dose of about 100 mg/m² for each dose of etoposide.
64. The method, use, or glofitamab for use of embodiment 62 or 63, wherein ifosfamide and carboplatin are administered on Day 2 of the first and second dosing cycles and the C1D1-C1D3 and C2D1-C2D3 of etoposide are administered on Days 1, 2, and 3, respectively, of each of the first and second dosing cycles.
65. The method, use, or glofitamab for use of any one of embodiments 53 to 64, wherein the first and second dosing cycles are each 21-day dosing cycles.
66. The method, use, or glofitamab for use of any one of embodiments 53 to 65, wherein the dosing regimen comprises one or more additional dosing cycles.
67. The method, use, or glofitamab for use of embodiment 66, wherein the one or more additional dosing cycles are each 21-day dosing cycles.
68. The method, use, or glofitamab for use of embodiment 66 or 67, wherein the dosing regimen comprises three dosing cycles in total.
69. The method, use, or glofitamab for use of embodiment 66 or 67, wherein the dosing regimen comprises four or five dosing cycles in total.
70. The method, use, or glofitamab for use of any one of embodiments 66 to 69, wherein the one or more additional dosing cycles comprises a third dosing cycle, and wherein the third dosing cycle comprises:
   (a) an additional single dose of glofitamab;
   (b) an additional single dose of rituximab; and
   (c) an additional single dose of ifosfamide; an additional single dose of carboplatin; and an additional first dose, an additional second dose, and an additional third dose of etoposide.
71. The method, use, or glofitamab for use of embodiment 70, wherein the additional single dose of glofitamab is about 30 mg.
72. The method, use, or glofitamab for use of embodiment 70 or 71, wherein the additional single dose of glofitamab is administered to the individual on Day 8 of the third dosing cycle.
73. The method, use, or glofitamab for use of embodiment 70, wherein the additional single dose of rituximab is about 375 mg/m².
74. The method, use, or glofitamab for use of embodiment 73, wherein the additional single dose of rituximab is administered on Day 1 of each of the third dosing cycle.
75. The method, use, or glofitamab for use of any one of embodiments 70 to 74, wherein ifosfamide is administered at a dose of about 5000 mg/m², carboplatin is administered at a dose in mg to target area under the curve (AUC) of about 5 mg/mL/min with maximum dose of about 750 mg, and etoposide is administered at a dose of about 100 mg/m² for each dose of etoposide.
76. The method, use, or glofitamab for use of any one of embodiments 70 to 75, wherein ifosfamide and carboplatin are administered on Day 2 of each of the one or more additional dosing cycles and the additional first dose, the additional second dose, and the additional third dose of etoposide are administered on Days 1, 2, and 3, respectively, of the third dosing cycle.
77. The method, use, or glofitamab for use of any one of embodiments 66, 67, and 69-76, wherein the one or more additional dosing cycles comprises a fourth dosing cycle or a fourth and a fifth dosing cycle, and wherein the fourth or fifth dosing cycle each comprises an additional single dose of glofitamab.
78. The method, use, or glofitamab for use of embodiment 77, wherein the single dose of glofitamab is about 30 mg.
79. The method, use, or glofitamab for use of embodiment 77 or 78, wherein the single dose of glofitamab is administered on Day 1 of the fourth or fifth dosing cycle.
80. The method, use, or glofitamab for use of any one of embodiments 1 to 79, wherein the control treatment comprises administering a single dose of rituximab, a single dose of ifosfamide, a single dose of carboplatin, and a first dose, a second dose, and a third dose of etoposide according to a dosing regimen comprising three 21-day dosing cycles.
81. The method, use, or glofitamab for use of embodiment 80, wherein rituximab is administered at a dose of about 375 mg/m², ifosfamide is administered at a dose of about 5000 mg/m², carboplatin is administered at a dose in mg to target area under the curve (AUC) of about 5 mg/mL/min with maximum dose of about 800 mg, and etoposide is administered at a dose of about 100 mg/m² for each dose of etoposide.
82. The method, use, or glofitamab for use of embodiment 80 or 81, wherein rituximab is administered on Day 1 of each dosing cycle, ifosfamide and carboplatin are administered on Day 2 of each of dosing cycle, and the first dose, the second dose, and the third dose of etoposide are administered on Days 1, 2, and 3, respectively, of each dosing cycle.
83. The method, use, or glofitamab for use of any one of embodiments 1 to 82, wherein the individual has received one prior systemic therapy.
84. The method, use, or glofitamab for use of any one of embodiments 1 to 83, wherein the individual is transplant or CAR-T cell therapy eligible.
85. The method, use, or glofitamab for use of embodiment 84, wherein the treatment comprising glofitamab, rituximab, ifosfamide, carboplatin, and etoposide is administered as salvage treatment prior to CAR-T cell therapy.
86. The method, use, or glofitamab for use of any one of embodiments 1 to 85, wherein the DLBCL is not otherwise specified (NOS) or high-grade B-cell lymphoma (HGBCL).
87. The method, use, or glofitamab for use of embodiment 86, wherein the HGBCL is HGBCL with MYC and BCL-2/6 rearrangements.
88. The method, use, or glofitamab for use of embodiment 86, wherein the HGBCL is HGBCL NOS.
89. The method, use, or glofitamab for use of any one of embodiments 1 to 88, wherein the individual has extranodal disease.
90. A method for treating relapsed or refractory DLBCL in a human individual in need thereof, comprising administering to the human individual an effective amount of glofitamab, rituximab, ifosfamide, carboplatin, and etoposide, wherein the individual achieves a CR.
91. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein the individual achieves a CR.
92. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein the individual achieves a CR.
93. A method for treating relapsed or refractory DLBCL in a human individual in need thereof, comprising administering to the human individual an effective amount of glofitamab, rituximab, ifosfamide, carboplatin, and etoposide, wherein the individual achieves an OR.
94. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein the individual achieves a OR.
95. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein the individual achieves a OR.
96. A method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a cytokine release syndrome (CRS) event rate of about 50%, about 57%, or about 62%.
97. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a cytokine release syndrome (CRS) event rate of about 50%, about 57%, or about 62%.
98. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a cytokine release syndrome (CRS) event rate of about 50%, about 57%, or about 62%.
99. A method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 2 or higher CRS event rate of about 20%, 21%, or 23%, wherein the CRS event is graded according to the American Society for Transplantation and Cellular Therapy (ASTCT) consensus grading system (Lee et al., Biol Blood Marrow Transplant, 25(4): 625-638, 2019).
100. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a Grade 2 or higher CRS event rate of about 20%, 21%, or 23%, wherein the CRS event is graded according to the American Society for Transplantation and Cellular Therapy (ASTCT) consensus grading system (Lee et al., Biol Blood Marrow Transplant, 25(4): 625-638, 2019).
101. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a Grade 2 or higher CRS event rate of about 20%, 21%, or 23%, wherein the CRS event is graded according to the American Society for Transplantation and Cellular Therapy (ASTCT) consensus grading system (Lee et al., Biol Blood Marrow Transplant, 25(4): 625-638, 2019).
102. A method for treating relapsed or refractory DLBCL in a human individual in need thereof, comprising administering to the human individual an effective amount of glofitamab, rituximab, ifosfamide, carboplatin, and etoposide, wherein individual does not experience a Grade 3 or higher CRS event, wherein the CRS event is graded according to the ASTCT consensus grading system.
103. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein individual does not experience a Grade 3 or higher CRS event, wherein the CRS event is graded according to the ASTCT consensus grading system.
104. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein individual does not experience a Grade 3 or higher CRS event, wherein the CRS event is graded according to the ASTCT consensus grading system.
105. A method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a serious adverse event (SAE) rate of about 39.5%, 41.5 or 45.2%.
106. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a serious adverse event (SAE) rate of about 39.5%, 41.5 or 45.2%.
107. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a serious adverse event (SAE) rate of about 39.5%, 41.5 or 45.2%.
108. A method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in a Grade 3 or 4 adverse event (AE) event rate of about 60.5%.
109. Glofitamab for use in a method of treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a Grade 3 or 4 adverse event (AE) event rate of about 60.5%.
110. Use of glofitamab in the manufacture of a medicament for treating relapsed or refractory DLBCL in a human individual in need thereof, wherein a treatment comprising an effective amount of glofitamab, rituximab, ifosfamide, carboplatin, and etoposide is to be administered to the human individual, wherein administering such treatment to a plurality of human individuals results in a Grade 3 or 4 adverse event (AE) event rate of about 60.5%.
111. The method, use, or glofitamab for use of any one of embodiments 1 to 110, wherein the method further comprises administering to the individual one or more additional therapeutic agents.
112. The method, use, or glofitamab for use of embodiment 111, wherein the one or more additional therapeutic agent is tocilizumab.
113. The method, use, or glofitamab for use of embodiment 112, wherein tocilizumab is administered intravenously at a dose of about 8 mg/kg, not exceeding 800 mg.
114. The method, use, or glofitamab for use of embodiment 111, wherein the one or more additional therapeutic agents is a corticosteroid.
115. The method, use, or glofitamab for use of embodiment 114, wherein the corticosteroid comprises prednisone, prednisolone, methylprednisolone, or dexamethasone.
116. The method, use, or glofitamab for use of embodiment 115, wherein the corticosteroid is dexamethasone.
117. The method, use, or glofitamab for use of embodiment 116, wherein dexamethasone is administered intravenously at a dose of about 20 mg at least about one hour prior to the administration of any dose of the glofitamab or the obinutuzumab.
118. The method, use, or glofitamab for use of embodiment 115, wherein the corticosteroid is methylprednisolone.
119. The method, use, or glofitamab for use of embodiment 118, wherein methylprednisolone is administered intravenously at a dose of about 80 mg at least about one hour prior to the administration of any dose of the glofitamab or the obinutuzumab.
120. The method, use, or glofitamab for use of embodiment 119, wherein the corticosteroid is prednisone or prednisolone.
121. The method, use, or glofitamab for use of embodiment 120, wherein prednisone or prednisolone is administered orally or intravenously at a dose of about 100 mg at least about one hour prior to the administration of any dose of the glofitamab or the obinutuzumab.
122. The method, use, or glofitamab for use of embodiment 111, wherein the one or more additional therapeutic agents is an antihistamine.
123. The method, use, or glofitamab for use of embodiment 122, wherein the antihistamine is diphenhydramine.
124. The method, use, or glofitamab for use of embodiment 123, wherein diphenhydramine is administered orally or intravenously at a dose of about 50 mg.
125. The method, use, or glofitamab for use of embodiment 124, wherein diphenhydramine is administered at least about 30 minutes prior to the administration of any dose of the glofitamab or the obinutuzumab.
126. The method, use, or glofitamab for use of embodiment 111, wherein the one or more additional therapeutic agents comprises granulocyte-colony stimulating factor (G-CSF).
127. The method, use, or glofitamab for use of embodiment 126, wherein G-CSF is administered between about one day and about two days after administration of any dose of rituximab, ifosfamide, carboplatin, and/or etoposide.
128. The method, use, or glofitamab for use of embodiment 127, wherein G-CSF is administered intravenously or subcutaneously at a dose of about 5 μg/kg/day or about 10 μg/kg/day.
129. The method, use, or glofitamab for use of embodiment 128, wherein G-CSF is administered at a dose of about 5 μg/kg/day in the first dosing cycle and about 10 μg/kg/day in the second dosing cycle and/or each additional dosing cycle.
130. The method, use, or glofitamab for use of embodiment 111, wherein the one or more additional therapeutic agents is an antipyretic.
131. The method, use, or glofitamab for use of embodiment 130, wherein the antipyretic is acetaminophen or paracetamol.
132. The method, use, or glofitamab for use of embodiment 131, wherein acetaminophen or paracetamol is administered orally or intravenously at a dose of between about 500 to about 1000 mg.
133. The method, use, or glofitamab for use of embodiment 132, wherein acetaminophen or paracetamol is administered at least about 30 minutes prior to the administration of any dose of the glofitamab or the obinutuzumab.
134. The method, use, or glofitamab for use of embodiment 111, wherein the one or more additional therapeutic agents is mesna.
135. The method, use, or glofitamab for use of embodiment 134, wherein mesna is administered intravenously at a dose of about 5000 mg/m².
136. The method, use, or glofitamab for use of embodiment 135, wherein mesna is administered via continuous infusion over about 24 hours on Day 3 of the first dosing cycle, on Day 6 of the second dosing cycle, and/or on Day 6 of each additional dosing cycle.
137. The method, use, or glofitamab for use of any one of embodiments 1 to 61, wherein the first dosing cycle comprises a first dose (C1D1) of ifosfamide, a second dose (C1D2) of ifosfamide, and a third dose (C1D3) of ifosfamide; a first dose (C1D1) of mesna, a second dose (C1D2) of mesna, and a third dose (C1D3) of mesna; a single dose (C1D1) of carboplatin; and a first dose (C1D1) of etoposide, a second dose (C1D2) of etoposide, and a third dose (C1D3) of etoposide; and
wherein the second cycle comprises a first dose (C2D1) of ifosfamide, a second dose (C2D2) of ifosfamide, and a third dose (C2D3) of ifosfamide; a first dose (C2D1) of mesna, a second dose (C2D2) of mesna, and a third dose (C2D3) of mesna; a single dose (C2D1) of carboplatin; and a first dose (C2D1) of etoposide, a second dose (C2D2) of etoposide, and a third dose (C2D3) of etoposide.
138. The method, use, or glofitamab for use of embodiment 137, wherein ifosfamide is administered at a dose of about 1666 mg/m² for each dose of ifosfamide, mesna is administered at a dose of about 1666 mg/m² for each dose of mesna, carboplatin is administered at a dose in mg to target area under the curve (AUC) of about 5 mg/mL/min with maximum dose of about 800 mg, and etoposide is administered at a dose of about 100 mg/m² for each dose of etoposide.
139. The method, use, or glofitamab for use of embodiment 137 or 138, wherein carboplatin is administered on Day 2 of the first and second dosing cycles and the C1D1-C1D3 and C2D1-C2D3 of ifosfamide, mesna, and etoposide are administered on Days 1, 2, and 3, respectively, of each of the first and second dosing cycles.
140. The method, use, or glofitamab for use of any one of embodiments 137 to 139, wherein the first and second dosing cycles are each 21-day dosing cycles.
141. The method, use, or glofitamab for use of any one of embodiments 137 to 140, wherein the dosing regimen comprises one or more additional dosing cycles.
142. The method, use, or glofitamab for use of embodiment 141, wherein the one or more additional dosing cycles are each 21-day dosing cycles.
143. The method, use, or glofitamab for use of embodiment 141 or 142, wherein the dosing regimen comprises three dosing cycles in total.
144. The method, use, or glofitamab for use of any one of embodiments 141 to 143, wherein the one or more additional dosing cycles each comprises:
   (a) an additional single dose of glofitamab;
   (b) an additional single dose of rituximab; and
   (c) an additional first dose, an additional second dose, and an additional third dose of ifosfamide; an additional first dose, an additional second dose, and an additional third dose of mesna; an additional single dose of carboplatin; and an additional first dose, an additional second dose, and an additional third dose of etoposide.
145. The method, use, or glofitamab for use of embodiment 144, wherein the additional single dose of glofitamab is about 30 mg.
146. The method, use, or glofitamab for use of embodiment 144 or 145, wherein the additional single dose of glofitamab is administered to the individual on Day 8 of each of the one or more additional dosing cycles.
147. The method, use, or glofitamab for use of embodiment 144, wherein the additional single dose of rituximab is about 375 mg/m².
148. The method, use, or glofitamab for use of embodiment 147, wherein the additional single dose of rituximab is administered on Day 1 of each of the one or more additional dosing cycles.
149. The method, use, or glofitamab for use of any one of embodiments 144 to 148, wherein ifosfamide is administered at a dose of about 1666 mg/m² for each dose of ifosfamide, mesna is administered at a dose of about 1666 mg/m² for each dose of mesna, carboplatin is administered at a dose in mg to target area under the curve (AUC) of about 5 mg/mL/min with maximum dose of about 750 mg, and etoposide is administered at a dose of about 100 mg/m² for each dose of etoposide.
150. The method, use, or glofitamab for use of any one of embodiments 144 to 149, wherein carboplatin is administered on Day 2 of each of the one or more additional dosing cycles and the additional first dose, the additional second dose, and the additional third dose of ifosfamide, mesna, and etoposide are administered on Days 1, 2, and 3, respectively, of each of the one or more additional dosing cycles.
151. The method, use, or glofitamab for use of any one of embodiments 137 to 150, wherein rituximab, ifosfamide, mesna, carboplatin, and etoposide are administered in an outpatient setting.
152. The method, use, or glofitamab for use of any one of embodiments 135 to 151, wherein each dose of mesna is administered simultaneously with the corresponding dose of ifosfamide on any day of a dosing cycle comprising a dose of mesna and a dose of ifosfamide.
153. The method, use, or glofitamab for use of any one of embodiments 1 to 152, wherein the individual is aged 18 years or older.
154. The method, use, or glofitamab for use of any one of embodiments 1 to 153, wherein glofitamab, rituximab, ifosfamide, mesna, carboplatin, and etoposide are administered to the induvial as salvage treatment prior to autologous stem cell transplant (ASCT) or CAR-T cell therapy.
155. In one embodiment, the invention features glofitamab for use in treating relapsed diffuse large B-cell lymphoma (DLBCL) in a patient eligible for autologous stem cell transplant or CAR-T cell therapy according to any of the embodiments above, wherein the patient has relapsed more than 12 months after receiving prior therapy, comprising 3 treatment cycles, wherein
a) Treatment cycle 1 comprises administering Obinutuzumab on day 1, administering ifosfamide, carboplatin, and etoposide or etoposide phosphate on days 1, 2 and 3, administering 2.5 mg glofitamab on day 8 and 10 mg glofitamab on day 15
b) Treatment cycles 2 and 3 comprise administering rituximab on day 1 of the respective cycle, administering ifosfamide, carboplatin, and etoposide or etoposide phosphate on days 1, 2 and 3 of the respective cycle and, administering 30 mg glofitamab on day 8 of the respective cycle.

### EXAMPLES

The following are examples of methods and compositions of the invention. It is understood that various other embodiments may be practiced, given the general description provided above.

### Example 1. A Phase IB, Open-Label, Multicenter, Single Arm Study Evaluating the Preliminary Efficacy, Safety, and Pharmacokinetics of Glofitamab in Combination with Rituximab Plus Ifosfamide, Carboplatin Etoposide Phosphate in Patients with Relapsed/Refractory Transplant or Car-T Therapy Eligible Diffuse B-Cell Lymphoma

### Study Rationale

The purpose of this study is to evaluate the preliminary efficacy, safety, and pharmacokinetics of glofitamab (glofit) in combination with rituximab plus ifosfamide, carboplatin, and etoposide (R-ICE) in participants with relapsed or refractory (R/R) diffuse large B-cell lymphoma (DLBCL), who have failed one prior line of therapy incorporating an anti-cluster of differentiation (CD) 20 antibody (i.e., rituximab) and an anthracycline, and who are transplant or CAR-T therapy eligible, defined as being medically eligible for intensive platinum-based salvage therapy followed by autologous stem cell transplantation (ASCT) or for CAR-T therapy.

### Objectives

The primary objective of this study is to evaluate the preliminary efficacy, safety, and pharmacokinetics of glofitamab in combination with R-ICE in participants with R/R DLBCL, who have failed one prior line of therapy incorporating an anti-CD20 antibody (i.e., rituximab) and an anthracycline, and who are transplant or CAR-T therapy eligible.

In this protocol, “study treatment” refers to the combination of treatments assigned to participants as part of this study (i.e., glofitamab and R-ICE). Specific objectives and corresponding endpoints for the study are outlined in Table 6.

TABLE 6
Objectives and Endpoints

| | |
|---|---|
| Objectives | Corresponding Endpoints |

Primary Objectives:

| | |
|---|---|
| To evaluate the preliminary | ORR after enrollment, defined as the |
| efficacy of glofit-R-ICE | proportion of participants that achieves a CR |
| | or PR within three cycles of glofit-R-ICE, as |
| | determined by the investigator according to |
| | Lugano criteria |

Secondary Objective:

| | |
|---|---|
| To evaluate the preliminary | EFS after enrollment, defined as the time from |
| efficacy of glofit-R-ICE | enrollment to the first occurrence of disease |
| | progression as determined by the investigator |
| | according to Lugano criteria, initiation of new anti- |
| | lymphoma therapy (not including planned ASCT |
| | or CAR-T therapy), or death from any cause |
| | (whichever occurs first) |
| | PFS after enrollment, defined as the time from |
| | enrollment to the first occurrence of disease |
| | progression or death from any cause (whichever |
| | occurs first), as determined by the investigator |
| | according to Lugano criteria |
| | MARR, defined as the proportion of participants |
| | treated with intent to proceed to ASCT that |
| | achieves a CR or PR within three cycles of glofit- |
| | R-ICE, as determined by the investigator |
| | according to Lugano criteria, and additionally |
| | achieves mobilization of a minimum of 2,000,000 |
| | CD34+ hematopoietic stem cells/kg for ASCT |
| | OS after enrollment, defined as the time from |
| | enrollment to death from any cause |
| | CR rate after enrollment, defined as the |
| | proportion of participants that achieves a CR |
| | within three cycles of glofit-R-ICE, as determined |
| | by the investigator according to Lugano criteria |
| | DOR, defined as the time from the first |
| | occurrence of a documented objective response |
| | (CR or PR) to disease progression or death from |
| | any cause (whichever occurs first), as determined |
| | by the investigator according to Lugano criteria |
| | DOCR, defined as the time from the first |
| | occurrence of a documented complete response |
| | to disease progression or death from any cause |
| | (whichever occurs first), as determined by the |
| | investigator according to Lugano criteria |
| To evaluate the safety of glofit-R-ICE | Incidence and severity of adverse events, with |
| | severity determined according to NCI CTCAE v5.0 |
| | Incidence and severity of cytokine release |
| | syndrome, with severity determined according to |
| | ASTCT 2019 criteria |
| | Change from baseline in targeted vital signs |
| | Change from baseline in targeted clinical |
| | laboratory test results |
| To establish the PK behavior of glofitamab | Maximum and Minimum Serum concentration of |
| when administered with R-ICE | glofitamab |
| To evaluate the incidence and prevalence of | Prevalence of ADAs against glofitamab at |
| ADAs to glofitamab when administered | baseline and incidence of ADAs during the study, |
| with R-ICE | and during follow up |

Exploratory Objective:

| | |
|---|---|
| To evaluate the effect of glofit-R-ICE on T- | T-cell activation and cytolytic function in vitro |
| cell function | |
| To evaluate potential relationships between | Relationship between serum concentration or |
| drug exposure and the efficacy and safety | PK parameters for glofitamab and efficacy |
| of glofit-R-ICE | endpoints |
| | Relationship between serum concentration or |
| | PK parameters for glofitamab and safety |
| | endpoints |
| To evaluate potential effects of ADAs | Relationship between ADA status and efficacy, |
| | safety, or PK endpoints |
| To identify and/or evaluate biomarkers that | Relationship between exploratory biomarkers in |
| are predictive of response to glofit-R-ICE | blood and tumor tissue and efficacy, safety, PK, |
| (i.e., predictive biomarkers), are early | immunogenicity, or other biomarker endpoints |
| surrogates of efficacy, are associated with | Time dependent kinetics of ctDNA and |
| progression to a more severe disease | associations with efficacy endpoints |
| state (i.e., prognostic biomarkers), are | |
| associated with acquired resistance to | |
| glofit-R-ICE, are associated with | |
| susceptibility to developing adverse | |
| events or can lead to improved adverse | |
| event monitoring or investigation, i.e., | |
| safety biomarkers), can provide evidence | |
| of glofit-R-ICE activity (i.e., | |
| pharmacodynamic biomarkers), or can | |
| increase the knowledge and | |
| understanding of disease biology and | |
| drug safety | |
| To evaluate features generated by deep | Concordance of aLugano and response |
| learning algorithms applied to PET-CT | assessed per 2014 Lugano Response Criteria by |
| images in patients treated with glofit-R- | Sponsor's radiologist |
| ICE, including automated total metabolic | Concordance of aTMTV and TMTV assessed by |
| tumor volume (aTMTV) (Jemaa et al. | Sponsor's radiologist\ |
| 2020) and automated response per 2014 | Concordance of aTMTV and TMTV assessed by |
| Lugano Response Criteria (aLugano). | Sponsor's radiologist |
| | Relationship between aTMTV and efficacy, |
| | safety, and biomarker endpoints |

ADA = anti-drug antibody; CAR-T = Chimeric Antigen Receptor T-cell; CR = Complete Response; CTCAE = Common Terminology Criteria for Adverse Events; ctDNA = circulating-tumor DNA; DOCR = duration of complete response; DOR = duration of response; EFS = event-free survival; glofit = glofitamab; MARR = mobilization adjusted response rate; ORR = objective response rate; OS = overall survival; PFS = progression-free survival; PK = pharmacokinetic; PR = partial response; R-ICE = Rituximab plus ifosfamide, carboplatin, etoposide NCI CTCAE = National Cancer Institute Common Terminology Criteria for Adverse Events.

### Overall Study Design

This is a Phase Ib, open-label, multicenter, single-arm trial in patients with R/R DLBCL who have failed one prior line of therapy incorporating an anti-CD20 antibody (i.e., rituximab) and an anthracycline, and who are transplant eligible, defined as being medically eligible for intensive platinum-based salvage therapy followed by ASCT or by CAR-T therapy.

Potential participants are screened for eligibility within a 28-day screening period. Approximately 40 participants will be enrolled, through an interactive voice or web-based response system (IxRS), to receive up to three 21-day cycles of glofit-R-ICE. During Cycle 1, participants receive obinutuzumab (Gazyva®) pretreatment (Gpt) on Day 1 plus ICE on Days 1-3, followed by step up dosing of glofitamab 2.5 mg on Day 8 and 10 mg on Day 15. During Cycles 2 and 3, participants receive R-ICE between Days 1-3 and glofitamab 30 mg on Day 8.

Participants are evaluated for response after two to three cycles of glofit-R-ICE, depending on institutional standard for number of cycles. Participants who do not have evidence of disease progression after two cycles may be treated with a third cycle of glofit-R-ICE at the investigator's discretion. Participants who experience progressive disease at any time point will discontinue study therapy.

Following completion of study therapy with glofit-R-ICE, participants who achieve CR or partial response proceed to ASCT or CAR-T therapy, with specific conditioning regimen and plan of care defined per institutional standard. Participants with stable disease or progressive disease receive further therapy as defined by their treating physician. Participants are followed for survival regardless of whether they proceed to transplant, CAR-T therapy or start new anti-lymphoma therapy.

Participants who complete glofit-R-ICE who are not yet able to undergo ASCT or CART therapy and who require additional maintenance therapy are permitted to receive up to two additional cycles of glofitamab monotherapy provided required conditions are met. In particular, in certain circumstances, a patient may not be able to proceed immediately to ASCT or CAR-T therapy and in the investigator's judgment may require additional maintenance therapy between glofit-R-ICE and ASCT or CAR-T. Such reasons include, but are not limited to, medical deconditioning after intensive chemotherapy or awaiting manufacture of CAR-T product. To facilitate maintenance of response in exceptional circumstances where patients are not able to immediately proceed to ASCT or CAR-T, administration of up to 2 cycles of glofitamab monotherapy is permitted after completion of glofit-R-ICE. Administration of additional glofitamab monotherapy is permissible provided all of the following conditions are met:
The patient has completed at least three cycles of glofit-R-ICE. If the patient has completed two cycles of glofit-R-ICE, the patient is only permitted proceed to monotherapy if the intent at study entry was to administer two cycles of therapy.
No additional cycles of ICE or other salvage therapies are planned.
The patient has radiographically confirmed CR or PR to glofit-R-ICE, and has not experienced PD.
The delay in ASCT/CAR-T is not due to adverse events that are potentially attributable to glofitamab.
The patient does not require repeat Gpt or step-up dosing due to a prolonged therapy delay.
The therapeutic intent continues to be that the patient proceeds to ASCT or CAR-T subsequent to completion of glofitamab.
Administration of glofitamab monotherapy is not expected to delay access to ASCT or CAR-T.
The investigator views that glofitamab monotherapy is in the patient's best interest, compared to other therapies.
   Glofitamab 30 mg as monotherapy is administered every three weeks for up to two doses, with the first dose at least three weeks after the most recent dose of glofitamab. The number of cycles is up to the investigator's discretion. Patients are required to permanently discontinue glofitamab monotherapy in the event of PD. 30 mg of glofitamab is administered on Day 1 of an optional fourth or fifth dosing cycle.

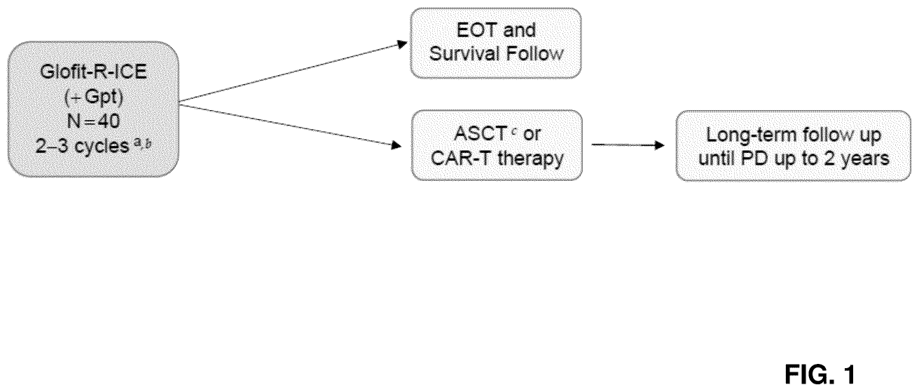

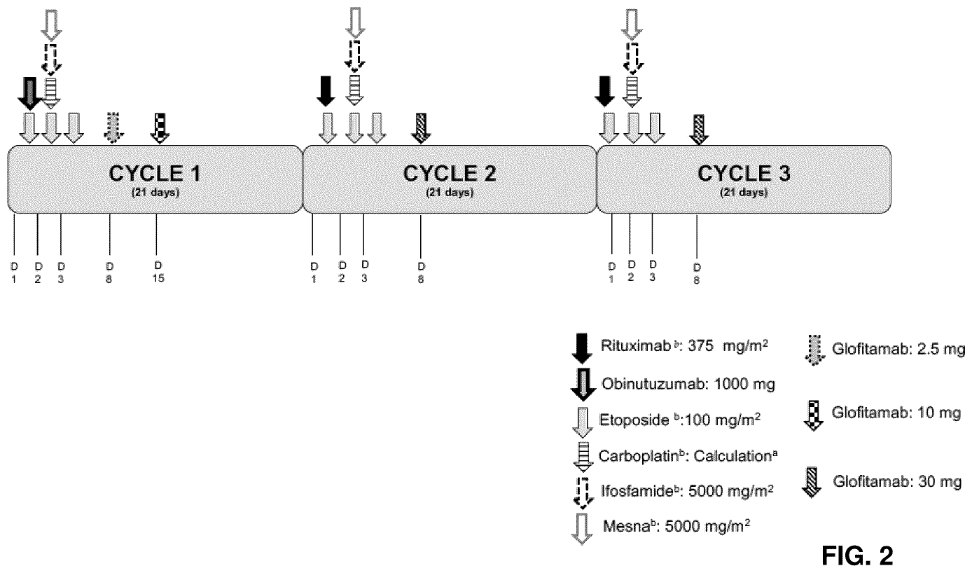

A study schema is provided in FIG. 1 and the dosing schema is shown in FIG. 2.

### Study Treatment

The investigational medicinal products (IMP) for this study are glofitamab (RO7082859, “glofit”), obinutuzumab (RO5072759), and tocilizumab (RO4877533).

### Glofitamab

Glofitamab is a bispecific antibody that binds to CD20 and CD3 comprising two Fab molecules which specifically bind to CD20 comprising the following six hypervariable regions (HVRs): HVR-H1: YSWIN (SEQ ID NO: 1); HVR-H2: RIFPGDGDTDYNGKFKG (SEQ ID NO:2); HVR-H3: NVFDGYWLVY (SEQ ID NO:3); HVR-L1: RSSKSLLHSNGITYLY (SEQ ID NO: 4); HVR-L2: QMSNLVS (SEQ ID NO: 5); and HVR-L3: AQNLELPYT (SEQ ID NO: 6) and one Fab molecule which specifically binds to CD3 comprising the following six hypervariable regions (HVRs): HVR-H1: TYAMN (SEQ ID NO: 9); HVR-H2: RIRSKYNNYATYYADSVKG (SEQ ID NO:10); HVR-H3: HGNFGNSYVSWFAY (SEQ ID NO: 11); HVR-L1: GSSTGAVTTSNYAN (SEQ ID NO: 12); HVR-L2: GTNKRAP (SEQ ID NO: 13); and HVR-L3: ALWYSNLWV (SEQ ID NO: 14).

Glofitamab is administered intravenously on a step-up dosing schedule, starting with 2.5 mg on Day 8 of Cycle 1 and 10 mg on Day 15 of Cycle 1, followed by 30 mg on Day 8 of Cycles 2 and 3.

Glofitamab should be administered to well-hydrated participants. Premedication with dexamethasone 20 mg IV is required and should be administered 1 hour prior to the administration of glofitamab; premedication with oral acetaminophen or paracetamol (500 or 1000 mg) and an antihistamine, such as diphenhydramine (50 mg), should be administered approximately 30 minutes prior to the start of the infusion.

Initially, glofitamab is administered to participants over 4 hours (±15 minutes) on Days 8 and 15 of Cycle 1 and Day 8 of Cycles 2 and 3. For participants who may be at an increased risk of CRS and participants who experienced CRS with their previous dose of glofitamab or who are, in the investigator's judgment, at increased risk of recurrent CRS with subsequent doses, the time of infusion may be extended to up to 8 hours. In the absence of infusion-related adverse events from rituximab or obinutuzumab administration, or CRS from glofitamab administration, the infusion time of glofitamab in Cycle 3 may be reduced to 2 hours (±15 minutes), at the discretion of the investigator.

### Obinutuzumab

Obinutuzumab pretreatment is administered by IV infusion as an absolute (flat) dose of 1000 mg on Day 1 of Cycle 1 for all participants. Obinutuzumab should be administered to well-hydrated participants.

Premedication with dexamethasone 20 mg IV or methylprednisolone 80 mg IV is required and should be administered 1 hour prior to the administration of obinutuzumab; premedication with oral acetaminophen or paracetamol (500 or 1000 mg) and an antihistamine, such as diphenhydramine (50 mg), should be administered approximately 30 minutes prior to the start of the infusion.

The obinutuzumab infusion may be split over 2 days if a participant is at increased risk for an infusion-related reaction (IRR) (e.g., because of high tumor burden, high peripheral lymphocyte count) or experiences an adverse event during the infusion.

When dexamethasone is required in the protocol, if dexamethasone is not available or if the participant has an intolerance to dexamethasone, methylprednisolone, prednisone, or prednisolone may be used. For a 20 mg IV dose of dexamethasone, an equivalent dose of 80 mg IV methylprednisolone, 100 mg prednisone PO, or 100 mg prednisolone by IV infusion may be used.

### Tocilizumab

Tocilizumab is administered as a rescue investigational medicinal product (IMP) when necessary to participants who experience a CRS event. Tocilizumab is administered at dose of 8 mg/kg for participants ≥30 kg and at dose of 12 mg/kg for participants <30 kg. Doses exceeding 800 mg per infusion are not recommended.

### Rituximab

Rituximab is administered intravenously at a dose of 375 mg/m² to participants on Day 1 of Cycles 2 and 3, along with ICE. Participants receive a total of 2 doses of rituximab if they complete all 3 cycles and 1 dose of rituximab if they complete just 2 cycles.

Rituximab preparation and administration are performed according to local prescribing information, including pre-medications. No subcutaneous administration of rituximab is allowed in the study. A locally approved standard of care biosimilar rituximab is permitted.

The infusion of rituximab may be split over 2 days if the participant is at increased risk for an infusion related reaction, IRR (high tumor burden or high peripheral lymphocyte count).

ICE Chemotherapy (Ifosfamide, Carboplatin, and Etoposide (with Mesna))

The ICE chemotherapy consists of IV ifosfamide, carboplatin, and etoposide. Administration is permitted to be inpatient or outpatient per investigator discretion. Etoposide phosphate may be substituted for etoposide at the same dose. Ifosfamide, carboplatin, and etoposide chemotherapy will be administered beginning Day 1 of Cycle 1 for two or three 21-day cycles, as follows:
Etoposide: 100 mg/m² IV daily on Day 1, 2, and 3 of each cycle
   Participants with creatinine clearance (CrCl)<50 mL/min should be dose reduced to 75% of planned dose (or lower per institutional standard).
Carboplatin: dose in mg=5 mg/mL/min×(25+CrCl), capped at 750 mg, IV infusion on Day 2 of each cycle
   CrCl should be calculated in mL/min using Cockcroft-Gault equation:

$$Males : CrCl ( mL / min ) = [ ( 140 - age ) \times ( weight in kg ) ] / [ 72 \times serum creatine ( mg / dL ) ]$$

$$Females : CrCl ( mL / min ) = 0.85 \times CrCl ( male )$$

Adjustments for body weight in overweight/obese participants (BMI ≥25) or for abnormally low creatinine are permitted per institutional standard.
Ifosfamide (inpatient administration): 5000 mg/m² mixed with mesna 5000 mg/m², continuous IV infusion over 24 hours starting on Day 2 of each cycle and ending on Day 3 of each cycle. Post-infusion mesna should be administered IV or PO according to institutional standard.
   Participants with CrCl <60 mL/min should be dose reduced to 80% of planned dose (or lower per institutional standard).
   Alternate dose for outpatient setting: Ifosfamide 1666 mg/m² mixed with mesna 1666 mg/m² IV daily on Day 1, 2, and 3 of each cycle is permitted if R-ICE is administered in the outpatient setting (Hertzberg et al. 2006, Ann Oncol 17, iv25-30).

In Cycle 1, obinutuzumab should be administered prior to ICE. Initiation of ICE (Cycle 1) is permitted to be delayed one day to facilitate completion of obinutuzumab administration.

For Cycles 2 and 3 rituximab should be administered prior to ICE. Initiation of ICE (Cycles 2 and 3) is permitted to be delayed 1 day if rituximab is administered 1 day prior to ICE per institutional standards or if there is a presentation of adverse events.

Drug administration and monitoring for drug-related toxicity (e.g., anaphylaxis, hemorrhagic cystitis, and neurotoxicity) should be conducted according to institutional standards.

### Granulocyte Colony-Stimulating Factor

All participants receive granulocyte colony-stimulating factor (G-CSF). Granulocyte colony-stimulating factor should be started 1-2 days after completion of R-ICE administration. Dosing of G-CSF should follow each site's institutional standards.

### Rationale for Primary Endpoint Selection

Given that this study is a Phase Ib study evaluating the preliminary efficacy of glofit-R-ICE in participants eligible for ASCT or CAR-T therapy, that the decision on moving forward or not with transplant depends on whether the participant's disease responds to the glofit-R-ICE combination, and that superior outcomes are observed in CAR-T therapy in patients with lower disease burden, objective response rate (ORR) is a clinically meaningful endpoint for assessing preliminary activity of glofit-R-ICE. The ORR is defined as the proportion of participants that achieves a CR or PR within three cycles of glofit-R-ICE, as determined by the investigator according to Lugano criteria.

### Rationale for Secondary Endpoint Selection

Secondary endpoints include standard endpoints used in DLBCL to assess long term survival-based endpoints, including EFS, PFS, and OS, as well as to assess duration of overall and complete response. Additionally, an endpoint for mobilization-adjusted response rate (MARR) is included to assess the percentage of patients who achieve an objective response as well as mobilization of the target dose of 2,000,000 CD34+ hematopoietic stem cells/kg typically required as a minimum for ASCT. The definition of MARR used in this study matches the study endpoint in the CORAL study (Gisselbrecht et al. 2010, J Clin Oncol 28, 4184-90). Although no minimum dose of CD34+ hematopoietic stem cells has been defined for ASCT, practice guidelines from the American Society of Blood and Marrow Transplantation notes that a dose of 2,000,000 CD34+ hematopoietic stem cells/kg is the “generally accepted minimum” number of stem cells required for ASCT (Duong et al. 2014, Biol Bone Marrow Transplant 20, 1262-73).

### Length of Study

The total duration of study participation for each individual is expected to be approximately 2 years and 9 months.

For patients proceeding to CAR-T therapy after completion of study treatment, leukapheresis preferably should occur prior to initiation of study treatment, as all patients underwent leukapheresis prior to R-ICE salvage on the TRANSFORM study (Kamdar et al. 2022) and as the effect of glofitamab+R-ICE on T-cell harvesting is unknown.

### Study Population

This study includes participants with DLBCL who have failed one prior line of therapy (incorporating an anti-CD20 antibody and an anthracycline) and who are transplant or CAR-T eligible.

### Inclusion Criteria

Participants are eligible to be included in the study only if all of the following criteria apply:
Participants who are age ≥18 years
Life expectancy ≥12 weeks
Histologically confirmed B-cell lymphoma, including one of the following diagnoses per the 2016 WHO classification of lymphoid neoplasms that occur either de novo or as transformed from indolent lymphomas:
   DLBCL, not otherwise specified (NOS) (including Epstein-Barr virus [EBV]+ DLBCL)
   High-grade B-cell lymphoma (HGBCL) with MYC and B-cell lymphoma 2 and/or B-cell lymphoma 6 rearrangements
   HGBCL, NOS
Treated with one line of prior systemic therapy including an anti-CD20 monoclonal antibody (i.e., rituximab) and an anthracycline
Relapsed or refractory disease after first-line chemoimmunotherapy
   Relapsed disease defined as complete remission to first-line therapy followed by biopsy-proven relapse
   Refractory disease defined as failure to achieve complete remission to first-line therapy, including:
      PD as best response to first-line therapy
      Stable disease (SD) as best response after at least 4 cycles of first-line therapy (e.g., 4 cycles of R-CHOP (rituximab, cyclophosphamide, doxorubicin, prednisone, rituximab and vincristine) or pola-R-CHP (polatuzumab vedotin, rituximab, cyclophosphamide, doxorubicin, and prednisone)
      PR as best response after at least 6 cycles and with either biopsy-proven residual disease or subsequent disease progression
Participant must be a candidate for high-dose chemotherapy followed by either ASCT or CAR-T therapy
At least one bi-dimensionally measurable (≥1.5 cm) nodal lesion, or one bi-dimensionally measurable ≥1 cm) extranodal lesion, as measured on CT scan
ECOG Performance Status of 0 or 1
Adequate hematologic function (unless attributable to the underlying disease, as established by extensive bone marrow involvement or associated with hypersplenism secondary to the involvement of the spleen by DLBCL per the investigator), defined as follows:

$$Hemoglobin \geq 9. g / dL ( \geq 90 g / L )$$

$$ANC \geq 1. \times 1 {0}^{9} / L ( \geq 1000 / μL )$$

Participants with history of benign ethnic neutropenia may be included with ANC ≥0.75×10⁹/L (≥750/μL).
Platelet count ≥75×10⁹/L (≥75,000/μL) without a transfusion in the week prior to starting study treatment.
Adequate renal function, defined as an estimated CrCl 45 mL/min

### Exclusion Criteria

Participants are excluded from the study if any of the following criteria apply:
Participant has been treated with more than one prior line of therapy for DLBCL.
   Treatment with both partial and complete frontline regimens (i.e., rituximab, cyclophosphamide, vincristine, and prednisone followed by R-CHOP or pola-R-CHP) will be counted as one line of therapy.
   Treatment with steroid monotherapy as a bridge to definitive therapy will not be counted as a prior line of therapy.
   Local therapies (e.g., radiotherapy) will not be considered as lines of therapy.
Primary mediastinal B-cell lymphoma (PMBCL)
Any history of Waldenström's macroglobulinemia
History of severe allergic or anaphylactic reactions to humanized or murine monoclonal antibodies (or recombinant antibody-related fusion proteins) or known sensitivity or allergy to murine products
Contraindication to obinutuzumab, rituximab, ifosfamide, mesna, carboplatin, etoposide, or tocilizumab
Prior treatment with glofitamab or other bispecific antibodies targeting both CD20 and CD3
Peripheral neuropathy assessed to be Grade >1 according to National Cancer Institute Common Terminology Criteria for Adverse Events (NCI CTCAE) v5.0 at enrollment
Treatment with radiotherapy, chemotherapy, immunotherapy, immunosuppressive therapy, or any investigational agent for the purposes of treating cancer within 2 weeks prior to first study treatment
Treatment with monoclonal antibodies for the purposes of treating cancer within 4 weeks prior to first study treatment
Primary or secondary CNS lymphoma at the time of enrollment or history of CNS lymphoma
Current or history of CNS disease, such as stroke, epilepsy, CNS vasculitis, or neurodegenerative disease
   Participants with a history of stroke who have not experienced a stroke or transient ischemic attack within the past 2 years and have no residual neurologic deficits, as judged by the investigator, are allowed.
Any of the following abnormal laboratory values, unless abnormal laboratory values are associated with the underlying lymphoma per the investigator:
   AST or ALT >2.5×upper limit of normal (ULN)
   Total bilirubin ≥1.5×ULN
   Participants with documented Gilbert disease may be enrolled if the total bilirubin is ≤3×ULN
   INR or PT >1.5×ULN in the absence of therapeutic anticoagulation
   PTT or aPTT >1.5×ULN in the absence of therapeutic anticoagulation or a lupus anticoagulant
History of other malignancy that could affect compliance with the protocol or interpretation of results, with the following exceptions:
   Participants with a history of curatively treated basal or squamous cell carcinoma of the skin or in situ carcinoma of the cervix at any time prior to enrollment are eligible.
   Participants with low-grade, early-stage prostate cancer (Gleason score 6 or below, Stage 1 or 2) with no requirement for therapy at any time prior to enrollment are eligible.
   Participants with any other malignancy appropriately treated with curative intent and the malignancy has been in remission without treatment for >2 years prior to enrollment are eligible.
   Participants receiving adjuvant endocrine therapy for non-metastatic, hormone receptor positive breast cancer for >2 years prior to enrollment are eligible.
Evidence of significant, uncontrolled concomitant diseases that could affect compliance with the protocol or interpretation of results, including significant cardiovascular disease (such as New York Heart Association Class III or IV or Objective Assessment Class C or D cardiac disease, myocardial infarction within the last 6 months, unstable arrhythmias, or unstable angina) or significant pulmonary disease (including obstructive pulmonary disease)
Known active bacterial, viral, fungal, mycobacterial, parasitic, or other infection (excluding fungal infections of nail beds) at study enrollment or any major episode of infection (as evaluated by the investigator) within 4 weeks prior to the first study treatment
Suspected or latent tuberculosis (confirmed by positive interferon-γ release assay)
Positive SARS-CoV-2 test within 7 days prior to enrollment. PCR or rapid antigen test result is acceptable.
Positive test results for hepatitis B virus (HBV) infection (defined as positive hepatitis B surface antigen [HBsAg] serology)
   Participants with occult or prior HBV infection (defined as negative HBsAg and positive hepatitis B core antibody [HBcAb]) may be included if HBV DNA is undetectable, provided that they are willing to undergo DNA testing on Day 1 of Cycles 2 and 3 and every three months for at least 12 months after the last cycle of study treatment and appropriate antiviral therapy.
Positive test results for hepatitis C virus (HCV) antibody
   Participants who are positive for HCV antibody are eligible only if polymerase chain reaction (PCR) is negative for HCV RNA.
Known or suspected chronic active Epstein-Barr viral infection (CAEBV)
   CAEBV is defined as a chronic illness lasting at least 3 months with an increased EBV level in either the tissue or the blood and lack of evidence of a known underlying immunodeficiency (Kimura and Cohen 2017). Participants should not be excluded for having an EBV+DLBCL if there is no other evidence or history suggestive of CAEBV.
Known or suspected history of hemophagocytic lymphohistiocytosis (HLH)
Known history of progressive multifocal leukoencephalopathy
Adverse events from prior anti-cancer therapy that have not resolved to Grade 1 or better (with the exception of alopecia and anorexia, or as otherwise permitted by inclusion criteria)
Administration of a live, attenuated vaccine within 4 weeks before first study treatment administration or anticipation that such a live, attenuated vaccine will be required during the study
Prior solid organ transplantation
Prior allogeneic stem cell transplant
Prior ASCT for lymphoma
Prior autologous stem cell transplant for any indication other than lymphoma, within 5 years from the start of study treatment
Active autoimmune disease requiring treatment.
   Participants with a history of autoimmune-related hypothyroidism on a stable dose of thyroid-replacement hormone are eligible.
   Participants with controlled Type 1 diabetes mellitus who are on an insulin regimen are eligible for the study.
   Participants with a history of autoimmune hepatitis, systemic lupus erythematosus, inflammatory bowel disease, vascular thrombosis associated with antiphospholipid syndrome, Wegener granulomatosis, Sjogren syndrome, multiple sclerosis, or glomerulonephritis will be excluded.
   Participants with a history of immune thrombocytopenic purpura, autoimmune hemolytic anemia, Guillain-Barre syndrome, myasthenia gravis, myositis, rheumatoid arthritis, vasculitis, or other autoimmune diseases will be excluded, unless they have not required systemic therapy in the last 12 months.
   Prior treatment with systemic immunosuppressive medications (including, but not limited to, cyclophosphamide, azathioprine, methotrexate, thalidomide, and anti-tumor necrosis factor agents), within 4 weeks prior to first dose of study treatment
   Ongoing corticosteroid use which in the opinion of the investigator, puts the patient at increased risk of steroid-related iatrogenic adrenal insufficiency
      The use of inhaled corticosteroids is permitted.
      The use of mineralocorticoids for management of orthostatic hypotension is permitted.
      The use of physiologic doses of corticosteroids for management of adrenal insufficiency is permitted.
   Recent major surgery (within 4 weeks before the first study treatment) other than for diagnosis
   Clinically significant history of cirrhotic liver disease
   Any other diseases, metabolic dysfunction, physical examination finding, or clinical laboratory finding giving reasonable suspicion of a disease or condition that contraindicates the use of an investigational drug or that may affect the interpretation of the results or renders the participant at high risk from treatment complications
   Pregnancy or breastfeeding, or intention of becoming pregnant during study treatment or within 18 months after the final dose of study treatment
      Women of childbearing potential must have a negative serum pregnancy test result within 7 days prior to initiation of study treatment.

### Example 2. Glofitamab in Combination with Rituximab Plus Ifosfamide, Carboplatin, and Etoposide Shows Favorable Efficacy and Manageable Safety in Patients with Relapsed or Refractory Diffuse Large B-Cell Lymphoma, Eligible for Stem Cell Transplant or Chimeric Antigen Receptor T-Cell Therapy: Results from a Phase Ib Study

### Background

Presented here are preliminary efficacy and safety data for glofitamab in combination with R-ICE (Glofit-R-ICE) in patients with R/R DLBCL eligible for ASCT or CAR T-cell therapy from a Phase Ib study (G043693; NCT05364424).

### Methods:

Enrolled patients had DLBCL not otherwise specified (NOS) or high-grade B-cell lymphoma (HGBCL), were treated with one prior line of therapy, had an Eastern Cooperative Oncology Group performance status of 0-1, and were eligible for ASCT or CAR T-cell therapy. Patients received obinutuzumab pretreatment (1000 mg) on Cycle (C) 1 Day (D) 1, followed by ICE (standard dose; C1D1-3) and R-ICE (R 375 mg/m² C2D1; ICE C2D1-3) in 21-day cycles. Glofitamab was administered by intravenous infusion with step-up dosing in C1 (D8: 2.5 mg; D15: 10 mg), and at target dose (30 mg) on C2D8. Patients could receive two or three cycles of Glofit-R-ICE (target dose) at the discretion of the investigator. The primary endpoint was investigator-assessed overall response rate (ORR), defined as the proportion of patients that achieved complete or partial metabolic response (CMR; PMR) within three cycles of Glofit-R-ICE. The efficacy-evaluable population was defined as all patients who received 21 dose of study drug and had 21 response assessment. The Glofit-R-ICE safety-evaluable population was defined as all patients who had 21 dose of Glofit-R-ICE.

### Results:

As of May 31, 2024, 29 patients provided informed consent and received study treatment. At baseline, median age was 65 (range: 41-77) and 72.4% of patients were male. Most patients were White (69.0%; Asian: 10.3%; Black/African American: 3.4%; Native Hawaiian: 3.4%; and 13.8% unknown). Histological subtypes were confirmed as DLBCL NOS (75.9%), HGBCL with MYC and BCL-2/6 rearrangements (10.3%), HGBCL NOS (3.4%), and 10.3% were unknown; 48.3% of patients had extranodal disease. Median duration of treatment was 23.0 days (range: 1-65). At the time of this interim analysis, the best overall response (CMR or PMR) was 75.9% (95% confidence interval [CI]: 56.5-89.7) and CMR rate was 65.5% (95% CI: 45.7-82.1) in the efficacy-evaluable population (n=29). In the Glofit-R-ICE-exposed population (n=38), cytokine release syndrome (CRS) was the most common adverse event (AE; 50.0% of patients [Grade 1, 28.9%; Grade 2, 21.1%]) with no Grade 23 events reported; all events resolved. No immune effector cell-associated neurotoxicity syndrome events were reported. Grade 3/4 AEs occurred in 60.5% of patients, with thrombocytopenia being the most common (28.9%). Infections occurred in 21.1% of patients (Grade 1, 5.3%; Grade 2, 7.9%; Grade 3, 7.9%). Serious AEs were reported in 39.5% patients. No Grade 5 AEs were reported. In total, one patient discontinued glofitamab treatment due to AEs. This patient had transformed Waldenstrom's macroglobulinemia and experienced Grade 2 CRS and Grade 3 tumor lysis syndrome.

### Conclusions:

Glofit-R-ICE demonstrated high overall and complete response rates in patients with R/R DLBCL eligible for ASCT or CAR T-cell therapy. The safety profile was manageable with a low treatment discontinuation rate.

### Example 3. Glofitamab in Combination with Rituximab Plus Ifosfamide, Carboplatin, and Etoposide Shows Favorable Efficacy and Manageable Safety in Patients with Relapsed or Refractory Diffuse Large B-Cell Lymphoma, Eligible for Stem Cell Transplant or Chimeric Antigen Receptor T-Cell Therapy: Results from a Phase Ib Study

### Introduction:

Presented here are preliminary efficacy and safety data for glofitamab in combination with R-ICE (Glofit-R-ICE) in patients with R/R DLBCL eligible for ASCT or CAR T-cell therapy from a Phase Ib study (G043693; NCT05364424).

### Methods:

Enrolled patients had DLBCL not otherwise specified (NOS) or high-grade B-cell lymphoma (HGBCL), had received one prior line of therapy, had an Eastern Cooperative Oncology Group performance status of 0-1, had no CNS lymphoma involvement, and were eligible for ASCT or CAR T-cell therapy. Patients received obinutuzumab pretreatment (1000 mg) on Cycle (C) 1 Day (D) 1, followed by ICE (standard dose; C1D1-3) and R-ICE (R 375 mg/m² C2D1; ICE C2D1-3) in 21-day cycles. Glofitamab was administered with step-up dosing in C1 (D8, 2.5 mg; D15, 10 mg) and at target dose (30 mg) on C2D8. Patients could receive two or three cycles of Glofit-R-ICE at target dose, at the discretion of the investigator. The primary endpoint was investigator-assessed ORR by 2014 Lugano criteria, defined as the proportion of patients that achieved complete or partial metabolic response (CMR; PMR) within three cycles of Glofit-R-ICE. The interim efficacy-evaluable population was defined as all patients who received their first dose of study treatment at least 77 days before May 31, 2024. The safety-evaluable population was defined as all patients who had 21 dose of any study treatment.

### Results:

As of May 31, 2024, 41 patients provided informed consent and received study treatment. At baseline, median age was 66 years (range: 41-78) and 71.1% of patients were male. Most patients were White (65.9%; Asian: 7.3%; Black/African American: 7.3%; Native Hawaiian: 2.4%; unknown: 12.2%; and 4.9% not reported). Histological subtypes were confirmed as DLBCL NOS (70.7%), HGBCL with MYC and BCL-2/6 rearrangements (12.2%), HGBCL NOS (4.9%), and DLBCL activated B cell (2.4%); 9.8% were unknown. Extranodal disease was reported in 39.0% of patients. Median duration of glofitamab treatment was 28.0 days (range: 1-65). At the time of this interim analysis, the best ORR (CMR or PMR) was 78.1% (95% confidence interval [CI]: 60.0-90.7) and the CMR rate was 68.8% (95% CI: 50.0-83.9) in the interim efficacy-evaluable population (n=32). In the safety-evaluable population (n=41), the most common adverse event (AE) was cytokine release syndrome (CRS; 48.8%; Grade [Gr] 1, 29.3%; Gr 2, 19.5%); no Gr 23 CRS events were reported and all events resolved. No immune effector cell-associated neurotoxicity syndrome events were reported. Infections occurred in 19.5% of patients (Gr 1, 4.9%; Gr 2, 7.3%; Gr 3, 7.3%). Gr 3/4 AEs occurred in 61.0% of patients; platelet count decreased/thrombocytopenia was the most common (26.8%). Serious AEs were reported in 41.5% of patients. No Gr 5 AEs were reported. In total, one patient discontinued glofitamab treatment due to AEs. This patient had transformed Waldenstrom's macroglobulinemia with Gr 2 CRS and Gr 3 tumor lysis syndrome.

### Conclusions:

Glofit-R-ICE demonstrated high overall and complete response rates in patients with R/R DLBCL eligible for ASCT or CAR T-cell therapy. The safety profile was manageable with a low treatment discontinuation rate.

### Example 4. Glofitamab in Combination with Rituximab Plus Ifosfamide, Carboplatin, and Etoposide Shows Favorable Efficacy and Manageable Safety in Patients with Relapsed or Refractory Diffuse Large B-Cell Lymphoma, Eligible for Stem Cell Transplant or Chimeric Antigen Receptor T-Cell Therapy: Results from a Phase Ib Study

### Introduction:

Diffuse large B-cell lymphoma (DLBCL), not otherwise specified, is the most common subtype of large B-cell lymphoma, with differences in prognosis reflecting heterogeneity in the pathological, molecular, and clinical features. High-risk Diffuse Large B-cell Lymphoma (DLBCL) is a more aggressive form of the disease, often characterized by specific genomic alterations like MYC/BCL2 translocations (double-hit lymphoma), high MYC and BCL2 protein expression (double expressor lymphoma), a high International Prognostic Index (IPI) score of 3-5, or a non-GCB (germinal center B-cell) subtype. A DLBCL relapse within 12 months of the initial treatment indicates early relapse, a high-risk situation with a generally poor prognosis compared to late relapses. Presented here are preliminary efficacy and safety data for glofitamab in combination with R-ICE (Glofit-R-ICE) in patients with R/R DLBCL eligible for ASCT or CAR T-cell therapy from a Phase Ib study (G043693; NCT05364424).

### Methods:

Enrolled patients had DLBCL not otherwise specified (NOS) or high-grade B-cell lymphoma (HGBCL), had received one prior line of therapy, had an Eastern Cooperative Oncology Group performance status (ECOG PS) of 0-1, had no CNS lymphoma involvement, and were eligible for ASCT or CAR T-cell therapy. Patients received obinutuzumab pretreatment (1000 mg) on Cycle (C) 1 Day (D) 1, followed by ICE (standard dose; C1D1-3) and R-ICE (R 375 mg/m² C2D1; ICE C2D1-3) in 21-day cycles. Glofitamab was administered with step-up dosing in C1 (D8, 2.5 mg; D15, 10 mg) and at target dose (30 mg) on C2D8. Patients could receive two or three cycles of Glofit-R-ICE at target dose, at the discretion of the investigator. The primary endpoint was investigator-assessed ORR by 2014 Lugano criteria, defined as the proportion of patients that achieved complete or partial metabolic response (CMR; PMR) within three cycles of Glofit-R-ICE. The interim efficacy-evaluable population was defined as all patients who received their first dose of study treatment at least 77 days before May 31, 2024. The safety-evaluable population was defined as all patients who had 21 dose of any study treatment.

### Results:

As of Aug. 19, 2024, 42 patients provided informed consent and received study treatment. Patient characteristics are provided below in Table 7.

TABLE 7
Patient Characteristics

| | |
|---|---|
| n (%)* | n = 42^{a} |

| Median age, years (range) | | 66 | (41-78) |
|---|---|---|---|
| ≥65 | | 24 | (57.1) |
| Male | | 29 | (69.0) |
| Ethnicity | Hispanic or Latino | 7 | (16.7) |
| | Not Hispanic or Latino | 28 | (66.7) |
| | Not stated | 6 | (14.3) |
| | Unknown | 1 | (2.4) |
| Race | Asian | 5 | (11.9) |
| | Black or African American | 3 | (7.1) |
| | Native Hawaiian | 1 | (2.4) |
| | Not reported | 2 | (4.8) |
| | White | 29 | (69.0) |
| | Unknown | 2 | (4.8) |
| ECOG PS | 0 | 13 | (31.0) |
| | 1 | 28 | (66.7) |
| | 2 | 1 | (2.4) |
| Ann Arbor stage III-IV | | 30 | (71.4) |
| Primary refractory at study entry^{b} | | 13 | (31.0) |
| Relapsed at study entry^{c} | | 29 | (69.0) |
| Relapsed within 12 months of prior therapy | | 8/29 | (27.6) |
| Relapsed ≥12 months of prior therapy | | 21/29 | (72.4) |
| Extranodal disease | | 16 | (38.1) |
| Bulky disease >6 cm | | 11 | (26.2) |
| Histology | DLBCL NOS | 30 | (71.4) |
| | HGBCL with MYC and BCL-2/6 | 5 | (11.9) |
| | HGBCL NOS | 2 | (4.8) |
| | DLBCL ABC | 1 | (2.4) |
| | Missing | 4 | (9.5) |
| Cell of | GCB | 20/35 | (47.6) |
| Origin^{d} | ABC | 12/35 | (28.6) |
| | Unclassified | 3/35 | (7.1) |
| | Missing | 7/35 | (16.7) |

*Unless otherwise stated.
^{a}One patient from the ITT population (N = 43) did not receive any study treatment.
^{b}Failure to achieve complete remission to first-line therapy.
^{c}Complete remission to first-line therapy followed by biopsy-proven relapse.
^{d}Determined from 35 evaluable samples. ABC, activated B-cell-like; GCB, germinal center B-cell-like.

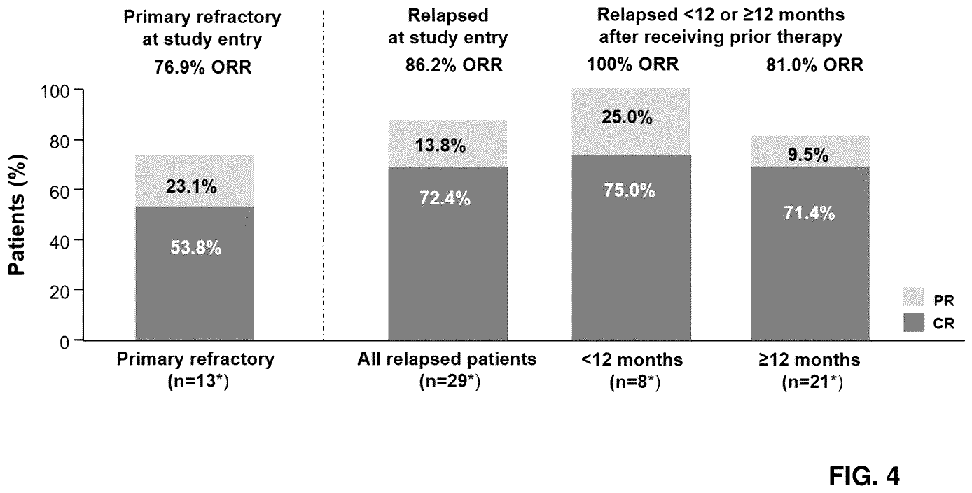

At the time of this interim analysis, the ORR (CR or PR) was 83.3% (OR achieved in 35 patients; 95% confidence interval [CI]: 68.6-93.0) and the CR rate was 66.7% (CR achieved in 28 patients; 95% CI: 50.5-80.4) in the interim efficacy-evaluable population (n=42). In patients who had primary refractory disease at study entry, 76.9% achieved an OR, of which 23.1% achieved a PR and 53.8% achieved a CR. In patients who had relapsed disease at study entry, 86.2% achieved an OR, of which 13.8% achieved a PR and 72.4% achieved a CR. In particular, in patients who had relapsed disease after receiving prior therapy for less than 12 months at study entry, 100.0% achieved an OR, of which 25.0% achieved a PR and 75.0% achieved a CR, while in patients who had relapsed disease after receiving prior therapy for greater than or equal to 12 months at study entry, 81.0% achieved an OR, of which 9.5% achieved a PR and 71.4% achieved a CR. FIG. 4 is a graph summarizing the response rates in patients with primary refractory or relapsed DLBCL.

The safety profile of the 42 safety-evaluable patients is shown below in Table 8.

TABLE 8
Safety Profile

| n (%) | n = 42* | |
|---|---|---|
| AE | | |
| All grades | 40 | (95.2)^{a} |
| Grade 3/4 | 30 | (71.4) |
| Grade 5 | 0 | |
| Serious AE | | |
| All grades | 19 | (45.2)^{a} |
| Grade 3/4 | 12 | (28.6) |
| Grade 5 | 0 | |
| AE leading to any treatment discontinuation | 4 | (9.5)^{b} |
| CRS^{c} | 24 | (57.1)^{a} |
| Grade 3/4 | 0 | |
| Thrombocytopenia^{d} | 23 | (54.8) |
| Grade 3/4 | 14 | (33.3) |
| Anemia | 20 | (47.6) |
| Grade 3/4 | 9 | (21.4) |
| Nausea | 15 | (35.7) |
| Grade 3/4 | 0 | |
| Neutropenia^{e} | 15 | (35.7) |
| Grade 3/4 | 12 | (28.5) |

*Safety-evaluable population: patients who had received ≥1 dose of any study drug; AEs were reported from C1D1 until 35 days after the final treatment, or the initiation of ASCT or CAR T-cell therapy; the last patient received initial treatment on Jun. 8, 2024.
^{a}Post-snapshot analysis included updated data from one investigation site (n = 1, updated from Gr 3 to Gr 1).
^{b}White blood cell count decreased (n = 2, Grade 2); nausea (n = 1, Grade 1); transformed Waldenstrom's macroglobulinemia with Grade 2 CRS, Grade 2 disseminated intravascular coagulation, Grade 3 acute kidney injury, Grade 3 liver injury, Grade 3 tumor lysis syndrome and Grade 3 decreased urine output (n = 1).
^{c}By ASTCT criteria (Lee, et al. Biol Blood Marrow Transplant 2019; 25: 625-38.).
^{d}Platelet count decreased and thrombocytopenia combined.
^{e}Neutrophil count decreased and neutropenia combined.
AE, adverse event; ASTCT, American Society for Transplantation and Cellular Therapy.

Among the safety-evaluable patients, 54.8% of patients completed 3 cycles of Glofit-R-ICE treatment. Glofit-R-ICE had a low treatment discontinuation rate due to AEs. No Grade 5 (fatal) AEs were reported.

The CRS event data are shown below in Table 9. In particular, 39 patients had received glofitamab treatment.

TABLE 9
Cytokine Release Syndrome Data

| CRS* | Glofit-exposed (n = 39) | |
|---|---|---|
| Any grade, n (%) | 24 | (61.5) |
| Grade 1 | 15 | (38.5) |
| Grade 2 | 9 | (23.1) |

Median time to CRS onset^{a}, days (range)

| | | |
|---|---|---|
| 2.5 mg Glofit (C1D8) | 2 | (1-5) |
| 10 mg Glofit (C1D15) | 1.5 | (1-3) |

Median CRS duration, days (range)

| | | |
|---|---|---|
| 2.5 mg Glofit (C1D8) | 2 | (1-4) |
| 10 mg Glofit (C1D15) | 2.5 | (1-4) |

CRS management, n (% of patients with CRS)

| | | |
|---|---|---|
| Tocilizumab | 13 | (54.2) |
| Corticosteroids | 3 | (12.5) |

*By ASTCT criteria.
^{a}C1D8: C1D1-14, C1D15: C1D15-21, C2D8: C2.

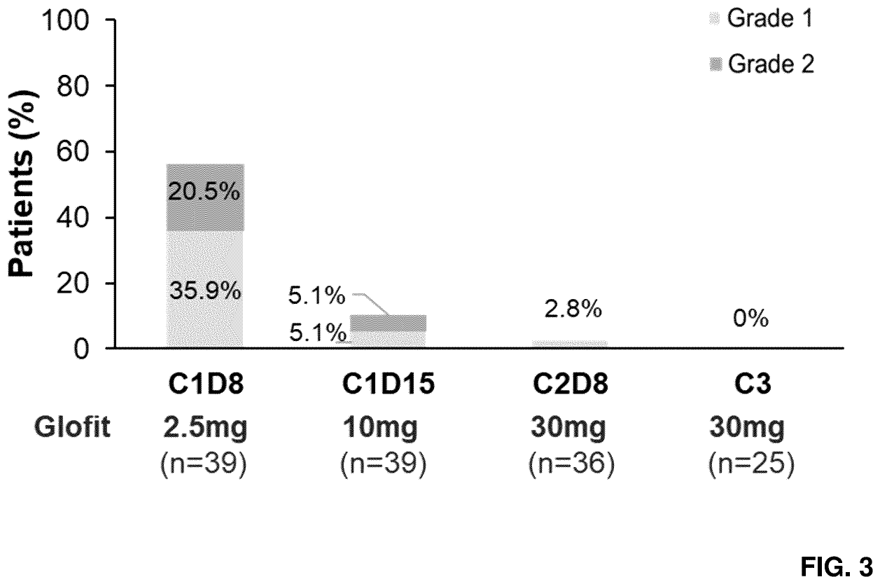

CRS events by dosing cycle and CRS ASTCT Grade are summarized in FIG. 3. CRS events were predominantly low-Grade and mostly occurred during C1. All CRS events resolved.

Other adverse events of clinical interest by highest grade are summarized below in Table 10.

TABLE 10
Other Adverse Events of Clinical Interest By Highest Grade

| n (%) | n = 42* | |
|---|---|---|
| Tumor flare | 1 | (2.4) |
| Grade 3 | 1 | (2.4) |
| Tumor lysis syndrome | 2 | (4.8) |
| Grade 3 | 2 | (4.8) |
| Serious infection^{a} | 2 | (4.8) |
| Grade 3 | 2 | (4.8) |
| Neutropenia^{b} | 15 | (35.7) |
| Grade 4 | 10 | (23.8) |
| Febrile neutropenia | 1 | (2.4) |
| Grade 3 | 1 | (2.4) |
| ICANS^{c} | Glofit-exposed (n = 39) | |
| Grade 1 | 2 | (5.1) |
| | 2 | (5.1) |

Safety-evaluable population: all patients who had received ≥1 dose of any study treatment.
^{a}Cellulitis (Grade 3, n = 1), and pneumonia (Grade 3, n = 1).
^{b}Neutrophil count decreased and neutropenia combined.
^{c}Two neurologic AEs were consistent with ICANS: n = 1 Grade 1 confusional state (serious), resolved after 17 days; n = 1 Grade 1 agitation (non-serious), resolved after 8 days; three reported neurological events were not consistent with ICANS: n = 2 Grade 3 syncopal events occurred during treatment with R-ICE prior to glofitamab exposure, n = 1 Grade 1 dysphonia. ICANS, immune effector cell-associated neurotoxicity syndrome.

### CONCLUSIONS

Fixed-duration glofitamab in combination with R-ICE demonstrated high response rates in patients with R/R DLBCL who were eligible for ASCT or CAR T-cell therapy. High responses were observed in patients with primary refractory or relapsed disease. Glofit-R-ICE had a manageable safety profile. CRS occurred mainly in C1, events were mostly low grade and all events resolved. Glofit-R-ICE had a low treatment discontinuation rate due to AEs. These data support further exploration of glofitamab-based combinations, including with R-ICE, in patients eligible for ASCT or CAR T-cell therapy.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating relapsed or refractory diffuse large B-cell lymphoma (DLBCL) in a human individual in need thereof, comprising administering to the human individual an effective amount of glofitamab, rituximab, ifosfamide, carboplatin, and etoposide,
wherein administering such treatment to a plurality of human individuals results in:
(a) an improvement in complete response (CR) rate of a plurality of human individuals as compared to a reference CR rate, wherein the reference CR rate is the CR rate of a plurality of human individuals who have received a control treatment comprising rituximab, ifosfamide, carboplatin, and etoposide in the absence of glofitamab;
(b) a CR rate of between 45% and 83%, or about 66%, 67%, or 69%;
(c) an improvement in overall response (OR) rate of a plurality of human individuals as compared to a reference OR rate, wherein the reference OR rate is the OR rate of a plurality of human individuals who have received a control treatment comprising rituximab, ifosfamide, carboplatin, and etoposide in the absence of glofitamab; and/or
(d) an OR rate of between 56% and 90%, or about 76%, 78%, or 83%.

2. The method of claim 1, wherein:
(a) the improvement in CR rate is;
(i) between 8% and 44%, or about 28%; and/or
(ii) statistically significant; or
(b) the improvement in OR rate is:
(i) between 1% and 29%, or about 15%; and/or
(ii) statistically significant.

3-6. (canceled)

7. The method of claim 1, wherein the CR rate is the proportion of individuals whose best overall response is a CR as determined by positron emission tomography-computed tomography (PET-CT) or positron emission tomography-magnetic resonance imaging (PET-MRI); and/or the OR rate is the proportion of individuals whose best overall response is a partial response (PR) or a CR.

8-14. (canceled)

15. The method of claim 1, wherein the treatment comprises administering glofitamab, rituximab, ifosfamide, carboplatin, and etoposide according to a dosing regimen comprising a first and a second dosing cycle, wherein:
(a) the first dosing cycle comprises a first dose (C1D1) of about 2.5 mg of the glofitamab and a second dose (C1D2) of about 10 mg of the glofitamab, and the second dosing cycle comprises a single dose (C2D1) of about 30 mg of the glofitamab; and
(b) the first dosing cycle comprises a single dose (C1D1) of ifosfamide; a single dose (C1D1) of carboplatin; and a first dose (C1D1) of etoposide, a second dose (C1D2) of etoposide, and a third dose (C1D3) of etoposide; and wherein the second cycle comprises a single dose (C2D1) of ifosfamide; a single dose (C2D1) of carboplatin; and a first dose (C2D1) of etoposide, a second dose (C2D2) of etoposide, and a third dose (C2D3) of etoposide.

16. The method of claim 15, wherein;
(a) the C1D1 and the C1D2 of the glofitamab are administered to the individual on Days 8 and 15, respectively, of the first dosing cycle;
(b) the C2D1 of the glofitamab is administered to the individual on Day 8 of the second dosing cycle;
(c) the first dosing cycle comprises a single dose (C1D1) of obinutuzumab;
(d) the second dosing cycle comprises a single dose (C2D1) of rituximab; and/or
(e) the first and second dosing cycles are each 21-day dosing cycles.

17-18. (canceled)

19. The method of claim 16, wherein;
(a) the C1D1 of obinutuzumab is about 1000 mg and/or is administered on Day 1 of the first dosing cycle;
(b) the C2D1 of rituximab is about 375 mg/m² and/or is administered on Day 1 of the second dosing cycle; and/or
(c) ifosfamide is administered at a dose of about 5000 mg/m², carboplatin is administered at a dose in mg to target area under the curve (AUC) of about 5 mg/mL/min with maximum dose of about 750 mg, and etoposide is administered at a dose of about 100 mg/m² for each dose of etoposide; and/or ifosfamide and carboplatin are administered on Day 2 of the first and second dosing cycles and the C1D1-C1D3 and C2D1-C2D3 of etoposide are administered on Days 1, 2, and 3, respectively, of each of the first and second dosing cycles.

20-27. (canceled)

28. The method of claim 15, wherein the dosing regimen comprises one or more additional dosing cycles.

29. The method of claim 28, wherein;
(a) the one or more additional dosing cycles are each 21-day dosing cycles;
(b) the dosing regimen comprises three, four, or five dosing cycles in total; and/or
(c) the one or more additional dosing cycles comprises a third dosing cycle, and wherein the third dosing cycle comprises:
(i) an additional single dose of glofitamab;
(ii) an additional single dose of rituximab; and
(iii) an additional single dose of ifosfamide; an additional single dose of carboplatin; and an additional first dose, an additional second dose, and an additional third dose of etoposide.

30-32. (canceled)

33. The method of claim 2, wherein;
(a) the additional single dose of glofitamab is about 30 mg; and/or is administered to the individual on Day 8 of the third dosing cycle;
(b) the additional single dose of rituximab is about 375 mg/m²; and/or is administered on Day 1 of each of the third dosing cycle; and/or
(c) the additional single dose of ifosfamide is about 5000 mg/m², the additional single dose of carboplatin is about 5 mg/mL/min in mg to target area under the curve (AUC) with maximum dose of about 750 mg, and the additional single dose of etoposide is about 100 mg/m² for each of the additional first dose, the additional second dose, and the additional third dose of etoposide; and/or the additional single dose of ifosfamide and the additional single dose of carboplatin are administered on Day 2 of the third dosing cycle and the additional first dose, the additional second dose, and the additional third dose of etoposide are administered on Days 1, 2, and 3, respectively, of the third dosing cycle.

34-38. (canceled)

39. The method of claim 28, wherein the one or more additional dosing cycles comprises a fourth dosing cycle or a fourth and a fifth dosing cycle, and wherein the fourth or fifth dosing cycle each comprises an additional single dose of glofitamab, wherein the single dose of glofitamab is about 30 mg and/or is administered on Day 1 of the fourth or fifth dosing cycle.

40-41. (canceled)

42. The method of claim 1, wherein the control treatment comprises administering a single dose of rituximab, a single dose of ifosfamide, a single dose of carboplatin, and a first dose, a second dose, and a third dose of etoposide according to a dosing regimen comprising three 21-day dosing cycles, wherein rituximab is administered at a dose of about 375 ma/m² on Day 1 of each dosing cycle; ifosfamide is administered at a dose of about 5000 ma/m² on Day 2 of each dosing cycle; and carboplatin is administered at a dose in ma to target area under the curve (AUC) of about 5 mg/mL/min with maximum dose of about 800 mg on Day 2 of each dosing cycle, and wherein etoposide is administered at a dose of about 100 mg/m² each on Days 1, 2, and 3, respectively, of each dosing cycle.

43-44. (canceled)

45. The method of claim 1, wherein:
(a) the individual has received one prior systemic therapy;
(b) the individual is transplant or CAR-T cell therapy eligible;
(c) the DLBCL is not otherwise specified (NOS) or high-grade B-cell lymphoma (HGBCL);
(d) the individual has extranodal disease; and/or
(e) the individual is aged 18 years or older.

46. (canceled)

47. The method of claim 45, wherein;
(a) the treatment comprising glofitamab, rituximab, ifosfamide, carboplatin, and etoposide is administered as salvage treatment prior to CAR-T cell therapy; and/or
(b) the HGBCL is HGBCL with MYC and BCL-2/6 rearrangements or HGBCL NOS.

48-55. (canceled)

56. A method for treating relapsed or refractory DLBCL in a human individual in need thereof, comprising administering to the human individual an effective amount of glofitamab, rituximab, ifosfamide, carboplatin, and etoposide, wherein the individual achieves a CR or an OR.

57. (canceled)

58. A method for treating relapsed or refractory DLBCL in a human individual in need thereof, comprising administering to the human individual an effective amount of glofitamab, rituximab, ifosfamide, carboplatin, and etoposide, wherein administering such treatment to a plurality of human individuals results in:
(a) a cytokine release syndrome (CRS) event rate of about 50%, about 57%, or about 62%;
(b) a Grade 2 or higher CRS event rate of about 20%, 21%, or 23%, wherein the CRS event is graded according to the American Society for Transplantation and Cellular Therapy (ASTCT) consensus grading system (Lee et al., Biol Blood Marrow Transplant, 25(4): 625-638, 2019);
(c) a serious adverse event (SAE) rate of about 39.5%, 41.5%, or 45.2%; and/or
(d) a Grade 3 or 4 adverse event (AE) event rate of about 60.5%.

59. (canceled)

60. A method for treating relapsed or refractory DLBCL in a human individual in need thereof, comprising administering to the human individual an effective amount of glofitamab, rituximab, ifosfamide, carboplatin, and etoposide, wherein individual does not experience a Grade 3 or higher CRS event, wherein the CRS event is graded according to the ASTCT consensus grading system.

61-62. (canceled)

63. The method of claim 1, wherein the method further comprises administering to the individual one or more additional therapeutic agents.

64. The method of claim 63, wherein the one or more additional therapeutic agent is:
(a) tocilizumab,
(b) a corticosteroid;
(c) an antihistamine;
(d) a granulocyte-colony stimulating factor (G-CSF);
(e) an antipyretic; and/or
(f) mesna.

65. The method of claim 64, wherein;
(a) tocilizumab is administered intravenously at a dose of about 8 mg/kg, not exceeding 800 mg;
(b) the corticosteroid comprises prednisone, prednisolone, methylprednisolone, or dexamethasone;
(c) the antihistamine is diphenhydramine;
(d) G-CSF is administered:
(i) between about one day and about two days after administration of any dose of rituximab, ifosfamide, carboplatin, and/or etoposide; and/or
(ii) intravenously or subcutaneously at a dose of about 5 μg/kg/day or about 10 μg/kg/day;
(e) the antipyretic is acetaminophen; and/or
(f) mesna is administered intravenously at a dose of about 5000 mg/m²; and/or mesna is administered via continuous infusion over about 24 hours on Day 3 of the first dosing cycle, on Day 6 of the second dosing cycle, and/or on Day 6 of each additional dosing cycle.

66-68. (canceled)

69. The method of claim 65, wherein;
(a) dexamethasone is administered intravenously at a dose of about 20 mg at least about one hour prior to the administration of any dose of the glofitamab or the obinutuzumab;
(b) methylprednisolone is administered intravenously at a dose of about 80 ma at least about one hour prior to the administration of any dose of the glofitamab or the obinutuzumab;
(c) prednisone or prednisolone is administered orally or intravenously at a dose of about 100 ma at least about one hour prior to the administration of any dose of the glofitamab or the obinutuzumab;
(d) diphenhydramine is administered:
(i) orally or intravenously at a dose of about 50 ma; and/or
(ii) at least about 30 minutes prior to the administration of any dose of the glofitamab or the obinutuzumab;
(e) G-CSF is administered at a dose of about 5 up/kg/day in the first dosing cycle and about 10 up/kg/day in the second dosing cycle and/or each additional dosing cycle;
(f) acetaminophen is administered:
(i) orally or intravenously at a dose of between about 500 to about 1000 ma; and/or
(ii) administered at least about 30 minutes prior to the administration of any dose of the glofitamab or the obinutuzumab; and/or
(g) each dose of mesna is administered simultaneously with the corresponding dose of ifosfamide on any day of a dosing cycle comprising a dose of mesna and a dose of ifosfamide.

70-88. (canceled)

89. The method of claim 1, wherein the first dosing cycle comprises a first dose (C1D1) of ifosfamide, a second dose (C1D2) of ifosfamide, and a third dose (C1D3) of ifosfamide; a first dose (C1D1) of mesna, a second dose (C1D2) of mesna, and a third dose (C1D3) of mesna; a single dose (C1D1) of carboplatin; and a first dose (C1D1) of etoposide, a second dose (C1D2) of etoposide, and a third dose (C1D3) of etoposide; and
wherein the second cycle comprises a first dose (C2D1) of ifosfamide, a second dose (C2D2) of ifosfamide, and a third dose (C2D3) of ifosfamide; a first dose (C2D1) of mesna, a second dose (C2D2) of mesna, and a third dose (C2D3) of mesna; a single dose (C2D1) of carboplatin; and a first dose (C2D1) of etoposide, a second dose (C2D2) of etoposide, and a third dose (C2D3) of etoposide.

90. The method of claim 89, wherein;
(a) ifosfamide is administered at a dose of about 1666 mg/m² for each dose of ifosfamide, mesna is administered at a dose of about 1666 mg/m² for each dose of mesna, carboplatin is administered at a dose in mg to target area under the curve (AUC) of about 5 mg/mL/min with maximum dose of about 800 mg, and etoposide is administered at a dose of about 100 mg/m² for each dose of etoposide;
(b) carboplatin is administered on Day 2 of the first and second dosing cycles and the C1D1-C1D3 and C2D1-C2D3 of ifosfamide, mesna, and etoposide are administered on Days 1, 2, and 3, respectively, of each of the first and second dosing cycles;
(c) the first and second dosing cycles are each 21-day dosing cycles:
(d) rituximab, ifosfamide, mesna, carboplatin, and etoposide are administered in an outpatient setting; and/or
(e) each dose of mesna is administered simultaneously with the corresponding dose of ifosfamide on any day of a dosing cycle comprising a dose of mesna and a dose of ifosfamide.

91-92. (canceled)

93. The method of claim 89, wherein the dosing regimen comprises one or more additional dosing cycles.

94. The method of claim 93, wherein:
(a) the one or more additional dosing cycles are each 21-day dosing cycles;
(b) the dosing regimen comprises three dosing cycles in total; and/or
(c) the one or more additional dosing cycles each comprises:
(i) an additional single dose of glofitamab;
(ii) an additional single dose of rituximab; and
(iii) an additional first dose, an additional second dose, and an additional third dose of ifosfamide; an additional first dose, an additional second dose, and an additional third dose of mesna; an additional single dose of carboplatin; and an additional first dose, an additional second dose, and an additional third dose of etoposide.

95-96. (canceled)

97. The method of claim 93, wherein;
(a) the additional single dose of glofitamab is about 30 mg; and/or is administered to the individual on Day 8 of each of the one or more additional dosing cycles;
(b) the additional single dose of rituximab is about 375 mg/m²; and/or is administered on Day 1 of each of the one or more additional dosing cycles;
(c) ifosfamide is administered at a dose of about 1666 mg/m² for each dose of ifosfamide, mesna is administered at a dose of about 1666 mg/m² for each dose of mesna, carboplatin is administered at a dose in mg to target area under the curve (AUC) of about 5 mg/mL/min with maximum dose of about 750 mg, and etoposide is administered at a dose of about 100 mg/m² for each dose of etoposide; and/or
(d) carboplatin is administered on Day 2 of each of the one or more additional dosing cycles and the additional first dose, the additional second dose, and the additional third dose of ifosfamide, mesna, and etoposide are administered on Days 1, 2, and 3, respectively, of each of the one or more additional dosing cycles.

98-104. (canceled)

105. The method of claim 56, wherein;
(a) the individual has received one prior systemic therapy;
(b) the individual is transplant or CAR-T cell therapy eligible;
(c) the DLBCL is not otherwise specified (NOS) or high-grade B-cell lymphoma (HGBCL);
(d) the individual has extranodal disease; and/or
(e) the individual is aged 18 years or older.

106. The method of claim 105, wherein;
(a) glofitamab, rituximab, ifosfamide, mesna, carboplatin, and etoposide are administered to the induvial individual as salvage treatment prior to autologous stem cell transplant (ASCT) or CAR-T cell therapy; and/or
(b) the HGBCL is HGBCL with MYC and BCL-2/6 rearrangements or HGBCL NOS.

107-108. (canceled)


SEQUENCE LISTING (57 sequences)

SEQ ID NO: 1 - protein, 5 aa, synthetic construct
YSWIN

SEQ ID NO: 2 - protein, 17 aa, synthetic construct
RIFPGDGDTDYNGKFKG

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGNSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNLWV

SEQ ID NO: 15 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 16 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 17 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
GGGGS

SEQ ID NO: 22 - protein, 5 aa, synthetic construct
SGGGG

SEQ ID NO: 23 - protein, 9 aa, synthetic construct
GGGGSGGGG

SEQ ID NO: 24 - protein, 20 aa, synthetic construct
GGGGSGGGGSGGGGSGGGGS

SEQ ID NO: 25 - protein, 12 aa, synthetic construct
GGGSGGGSGGGS

SEQ ID NO: 26 - protein, 16 aa, synthetic construct
GGGSGGGSGGGSGGGS

SEQ ID NO: 27 - protein, 20 aa, synthetic construct
GGGSGGGSGGGSGGGSGGGS

SEQ ID NO: 28 - protein, 24 aa, synthetic construct
GGGSGGGSGGGSGGGSGGGSGGGS

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 25 aa, synthetic construct
GGGGSGGGGSGGGGSGGGGSGGGGS

SEQ ID NO: 33 - protein, 13 aa, synthetic construct
GGGSGGGSGGGSG

SEQ ID NO: 34 - protein, 17 aa, synthetic construct
GGGSGGGSGGGSGGGSG

SEQ ID NO: 35 - protein, 21 aa, synthetic construct
GGGSGGGSGGGSGGGSGGGSG

SEQ ID NO: 36 - protein, 25 aa, synthetic construct
GGGSGGGSGGGSGGGSGGGSGGGSG

SEQ ID NO: 37 - protein, 11 aa, synthetic construct
GGGGSGGGGSG

SEQ ID NO: 38 - protein, 16 aa, synthetic construct
GGGGSGGGGSGGGGSG

SEQ ID NO: 39 - protein, 21 aa, synthetic construct
GGGGSGGGGSGGGGSGGGGSG

SEQ ID NO: 40 - protein, 26 aa, synthetic construct
GGGGSGGGGSGGGGSGGGGSGGGGSG

SEQ ID NO: 41 - protein, 14 aa, synthetic construct
GGGSGGGSGGGSGG

SEQ ID NO: 42 - protein, 18 aa, synthetic construct
GGGSGGGSGGGSGGGSGG

SEQ ID NO: 43 - protein, 22 aa, synthetic construct
GGGSGGGSGGGSGGGSGGGSGG

SEQ ID NO: 44 - protein, 26 aa, synthetic construct
GGGSGGGSGGGSGGGSGGGSGGGSGG

SEQ ID NO: 45 - protein, 12 aa, synthetic construct
GGGGSGGGGSGG

SEQ ID NO: 46 - protein, 17 aa, synthetic construct
GGGGSGGGGSGGGGSGG

SEQ ID NO: 47 - protein, 22 aa, synthetic construct
GGGGSGGGGSGGGGSGGGGSGG

SEQ ID NO: 48 - protein, 27 aa, synthetic construct
GGGGSGGGGSGGGGSGGGGSGGGGSGG

SEQ ID NO: 49 - protein, 15 aa, synthetic construct
GGGSGGGSGGGSGGG

SEQ ID NO: 50 - protein, 19 aa, synthetic construct
GGGSGGGSGGGSGGGSGGG

SEQ ID NO: 51 - protein, 23 aa, synthetic construct
GGGSGGGSGGGSGGGSGGGSGGG

SEQ ID NO: 52 - protein, 27 aa, synthetic construct
GGGSGGGSGGGSGGGSGGGSGGGSGGG

SEQ ID NO: 53 - protein, 13 aa, synthetic construct
GGGGSGGGGSGGG

SEQ ID NO: 54 - protein, 18 aa, synthetic construct
GGGGSGGGGSGGGGSGGG

SEQ ID NO: 55 - protein, 23 aa, synthetic construct
GGGGSGGGGSGGGGSGGGGSGGG

SEQ ID NO: 56 - protein, 28 aa, synthetic construct
GGGGSGGGGSGGGGSGGGGSGGGGSGGG

SEQ ID NO: 57 - protein, 11 aa, synthetic construct
DGGGGSGGGGS